(19) United States
(12) Patent Application Publication
(10) Pub. No.: US 2016/0039737 A1
(43) Pub. Date: Feb. 11, 2016

(54) USE OF SOLUBLE METAL SALTS IN METATHESIS REACTIONS

(71) Applicant: MATERIA, INC., Pasadena, CA (US)

(72) Inventors: Timothy M. CHAMPAGNE, Orange, CA (US); Thay A. UNG, Monrovia, CA (US)

(21) Appl. No.: 14/775,173

(22) PCT Filed: Mar. 14, 2014
(86) PCT No.: PCT/US14/27194

§ 371 Date: Sep. 11, 2015

Related U.S. Application Data
(60) Provisional application No. 61/785,708, filed on Mar. 14, 2013.

(51) Int. Cl. C07C 67/333; C07C 5/54; C07C 67/60

(52) U.S. Cl. CPC C07C 67/333; C07C 67/60; C07C 5/54

(57) ABSTRACT

The present invention describes the use of soluble metal salts to reduce impurities and metathesis catalysts poisons from olefinic feedstocks to improve olefin metathesis efficiency. The soluble metal salts were added to the olefinic feedstocks to prevent peroxides and catalyst poisons from inhibiting the metathesis catalyst. The soluble metal salts remain in the olefinic feedstocks and are used without further purification in the olefin metathesis reactions. The key to this invention is the soluble metal salt compounds do not inhibit the olefin metathesis catalysts but unexpectedly increase olefin metathesis catalyst efficiency while prior art heterogeneous metal complexes sequester the olefin metathesis catalyst, preventing olefin metathesis.

### RELATED APPLICATIONS

This application claims the benefit of U.S. Provisional Patent Application No. 61/785,708, filed Mar. 14, 2013 the contents of which is incorporated herein by reference.

### TECHNICAL FIELD

The present invention describes the use of soluble metal salts to reduce impurities and metathesis catalysts poisons from olefinic feedstocks to improve olefin metathesis efficiency. The soluble metal salts were added to the olefinic feedstocks to prevent peroxides and catalyst poisons from inhibiting the metathesis catalyst. The soluble metal salts remain in the olefinic feedstocks and are used without further purification in the olefin metathesis reactions. The key to this invention is the soluble metal salt compounds do not inhibit the olefin metathesis catalysts but unexpectedly increase olefin metathesis catalyst efficiency while prior art heterogeneous metal complexes sequester the olefin metathesis catalyst, preventing olefin metathesis.

### BACKGROUND

Olefin metathesis has emerged as a unique and powerful transformation for the interconversion of olefinic hydrocarbons, namely due to the development of well-defined catalysts. See Grubbs, R. H. Handbook of Metathesis, Wiley-VCH: Weinheim, Germany (2003). The exceptionally wide scope of substrates and functional group tolerances makes olefin metathesis a valuable technique that quickly and efficiently produces otherwise hard to make molecules, compared to traditional synthetic organic techniques. In particular, certain ruthenium and osmium carbene compounds known as “Grubbs catalysts,” have been identified as effective catalysts for olefin metathesis reactions such as, cross metathesis (CM), ring-closing metathesis (RCM), ring-opening metathesis (ROM), ring-opening cross metathesis (ROCM), ring-opening metathesis polymerization (ROMP) and acyclic diene metathesis (ADMET) polymerization. The use of ruthenium alkylidene complexes has greatly expanded the scope of this process due to increased tolerance of organic functionality, moisture, and oxygen. However, even with these advances, impurities, such as peroxides and catalyst poisons, in the olefinic substrates decrease the efficiency of the metathesis reaction by decreasing yields and requiring increased catalyst loadings. Metathesis catalysts are expensive; it is beneficial to remove impurities and catalyst poisons from metathesis reactions. Therefore there is a need for an efficient process to remove peroxides and catalyst poisons in the olefinic substrates, this invention addresses this need.

Solid adsorbents such as alumina, silica, zeolite molecular sieves, clays, and others have been used to reduce the peroxide value of internal olefins (U.S. Pat. No. 4,243,831), dicyclopentadiene (U.S. Pat. No. 4,584,425) and seed oil feedstocks (U.S. Pat. No. 7,745,652, U.S. Pat. No. 7,576,227, WO 03/093215). Reducing agents such as ethyl magnesium bromide, hydrogen lithium aluminum, n-butyl lithium, diethyl zinc, and others have been used to reduce the peroxide value of DCPD (U.S. Pat. No. 5,378,783). Reducing agents such as bisulfite and borohydride (WO 2008/009365) and thermal methods (heating to 200° C., WO 2009/020665) been used for the purification of seed oil feed stocks thereby improving the efficiency of metathesis reactions.

Hydrocarbyl phosphite (U.S. Pat. App. Pub. No. 2008/0132712) and phenol antioxidants (U.S. Pat. No. 3,873,466) were reported to reduce peroxide values but they differ from the present invention in that hydrocarbyl phosphite and phenol antioxidants are not soluble metal salts.

Fuerstner (JOC 2000, 65, 2204) reported using titanium (IV) isopropoxide in metathesis reactions (up to 30 wt %). In his applications, titanium (IV) isopropoxide acted as a Lewis acid to coordinate to a Lewis base. This reference does not describe using titanium (IV) isopropoxide to remove peroxides.

U.S. Pat. App. Pub. No. 2011/0160502 describes metallocene catalyzed polymerization of alpha olefins using methylalumoxane (MAO) as a co-activator. MAO is used to remove impurities from the feed streams. In addition, this reference describes ethenolysis in the presence of co-activators (MAO) but does not use metathesis.

Grubbs (U.S. Pat. No. 5,728,785) reported the ring opening polymerization of DCPD in the presence of sterically hindered peroxides. Upon post curing the peroxides decomposed to yield radical catalyzed crosslinking of the polymer. Preferred crosslinking agents are peroxides, such as t-butyl peroxide, 2,5-dimethyl-2,5-di-(tert-butylperoxy) hexyne-3, di-tert-butyl peroxide, and 2,5-dimethyl-2,5-di-(tert-butylperoxy) hexane or mixtures thereof. As our data will show, the peroxide value (PV) has a direct effect on inhibiting the metathesis reaction.

Many of these methods of purification include costly and time consuming filtration or secondary purification steps, and it would be advantageous to have an olefin treatment that is compatible with the metathesis catalysts.

The invention describes the use of soluble metal salts for the removal of peroxides and catalyst poisons from olefinic feedstock compositions. The invention is suitable for olefins including alpha-olefins and internal olefins. A preferred class of olefins this technique works well with seed oils, fatty acid methyl esters, alpha olefins, internal olefins, norbornene derivatives and cyclopentadiene derivatives.

The invention is also suitable for smaller molecules with one or more internal or terminal olefins. The treatment with a soluble metal salt reduces the peroxide levels resulting in improved olefin catalyst activity (e.g., higher turnover numbers). In addition to peroxides, other catalyst poisons may also be removed using this method and improve the efficiency of catalytic reactions.

The soluble metal salts serve as an alternative to heterogeneous treatments such as aluminas, silicas, clays, magnesias, sieves, and silicates already reported above. In applications where a metathesis catalyst and an olefin metathesis substrate are insensitive to the soluble metal salts, the homogeneous methodology of the invention is preferred.

### SUMMARY OF THE INVENTION

The invention is directed to addressing one or more of the aforementioned concerns and relates to the use of soluble metal salts to reduce impurities and metathesis catalysts poisons from olefinic feedstocks to improve olefin metathesis efficiency.

In one embodiment, the invention provides a composition comprising an olefinic feedstock, at least one soluble metal salt, and at least one olefin metathesis catalyst, wherein the olefinic feedstock comprises at least one natural seed oil.

In another embodiment, the invention provides a method for improving the olefin metathesis of an olefinic feedstock, comprising providing an olefinic feedstock, combining the olefinic feedstock with at least one soluble metal salt to form an olefinic feedstock composition, subjecting the olefinic feedstock composition to conditions effective to reduce the concentration of at least one impurity in the olefinic feedstock, combining the olefinic feedstock composition with at least one olefin metathesis catalyst, and subjecting the olefinic feedstock composition to conditions effective to promote an olefin metathesis reaction, wherein the olefinic feedstock comprises at least one olefin metathesis active compound derived from petroleum sources, fermentation sources, or natural sources such as oils extracted from plants or animals.

In another embodiment, the invention provides a method for improving the olefin metathesis of an olefinic feedstock, comprising providing an olefinic feedstock, combining the olefinic feedstock with at least one soluble metal salt to form an olefinic feedstock composition, subjecting the olefinic feedstock composition to conditions effective to reduce the concentration of at least one impurity in the olefinic feedstock, combining the olefinic feedstock composition with at least one olefin metathesis catalyst, and subjecting the olefinic feedstock composition to conditions effective to promote an olefin metathesis reaction.

In another embodiment, the invention provides a composition, comprising, an olefinic feedstock, at least one soluble metal salt, and at least one olefin metathesis catalyst, wherein the olefinic feedstock comprises at least one cyclic olefin,

In another embodiment, the invention comprises an article of manufacture comprising an olefinic feedstock, at least one soluble metal salt, and at least one olefin metathesis catalyst, where the olefinic feedstock comprises at least one natural seed oil.

In another embodiment, the invention provides an article of manufacture comprising an olefinic feedstock, at least one soluble metal salt, and at least one olefin metathesis catalyst, where the olefinic feedstock comprises at least one cyclic olefin.

In another embodiment, the invention provides a composition, comprising an olefinic feedstock, at least one soluble metal salt, and at least one olefin metathesis catalyst, wherein the olefinic feedstock comprises at least one olefin metathesis active compound derived from petroleum sources, fermentation sources, or natural sources such as oils extracted from plants or animals.

In another embodiment the invention provides an article of manufacture comprising an olefinic feedstock, at least one soluble metal salt, and at least one olefin metathesis catalyst, where the olefinic feedstock comprises at least one olefin metathesis active compound derived from petroleum sources, fermentation sources, or natural sources such as oils extracted from plants or animals.

### DETAILED DESCRIPTION OF THE INVENTION

### Terminology and Definitions

Unless otherwise indicated, the disclosure is not limited to specific reactants, substituents, catalysts, reaction conditions, or the like, as such may vary. It is also to be understood that the terminology used herein is for the purpose of describing particular embodiments only and is not intended to be limiting.

As used in the specification and the appended claims, the singular forms “a,” “an,” and “the” include plural referents unless the context clearly dictates otherwise. Thus, for example, reference to “an α-olefin” includes a single α-olefin as well as a combination or mixture of two or more α-olefins, reference to “a substituent” encompasses a single substituent as well as two or more substituents, and the like.

As used in the specification and the appended claims, the terms “for example,” “for instance,” “such as,” or “including” are meant to introduce examples that further clarify more general subject matter. Unless otherwise specified, these examples are provided only as an aid for understanding the applications illustrated in the present disclosure, and are not meant to be limiting in any fashion.

In this specification and in the claims that follow, reference will be made to a number of terms, which shall be defined to have the following meanings:

The term “olefinic feedstocks” as used herein refers to any olefin metathesis active compound containing at least one carbon-carbon double bond. These olefinic feedstocks may be derived from petroleum sources, fermentation processes, or from natural sources such as oils extracted from plants or animals. Examples of such petroleum-derived olefinic feedstocks include alpha-olefins, internal olefins and cyclic or polycyclic olefins such as, for example, DCPD (dicyclopentadiene), norbornenes, and substituted norbornenes. Examples of fermentation-derived olefinic feedstocks include fumarates, isobutylene, and various fatty acids and/or esters. Examples of olefinic feedstocks derived from natural sources include but are not limited to natural rubbers, terpenes and other isoprenoids including citronellene, linalool, myrcene, β-pinenes, pyrethrins, steroids, estolides, alkylresorcinols, cardanol, and fatty alcohols. Other examples of olefinic feedstocks derived from natural sources include various unsaturated and polyunsaturated triglyceride-based oils extracted from plants or animals. Such oils include natural seed oils, such as soybean oil (SBO), camelina oil, sunflower oil, canola oil, safflower oil, cottonseed oil, castor oil, rapeseed oil, peanut oil, corn oil, olive oil, palm oil, sesame oil, grape seed oil, and the fatty acid methyl esters derived therefrom. Such oils also include animal oils such as fish oils and tallow.

The term “soluble metal salt” as used herein refers to metal compounds which are liquid under reaction conditions, have sufficient solubility in the olefin metathesis substrate to reduce peroxides, and do not inhibit the metathesis catalyst, under reaction conditions. Typical reaction conditions include the soluble metal salt is mixed with the olefinic feedstock and stirred from 2 to 96 hours. The mixture may be heated slightly above room temperature, i.e., 30° C., or up to 80° C. The loading of the soluble metal salt may be from 0.1 wt % to 5 wt %, with preferred loadings of 0.5 wt % to 2.0 wt %. The soluble metal salt may be added directly to the olefin or as a solution in another solvent such as toluene.

The terms “soluble,” “solubility,” and the like as used herein refers to metal salts which have the ability to dissolve in the olefin metathesis substrate in any amount, preferably in 1 wt % to 5 wt %, with more preferred loadings of 0.5 wt % to 2.0 wt %. Any amount that will reduce peroxide levels and will not inhibit the metathesis catalyst, under reaction conditions, is acceptable.

The term “metal” as used herein refers to elements in the periodic table that consist of Transition Metal groups III to XII, Main group elements III, IV, and V, and the Lanthanides. Elements excluded from this metal definition include Boron, Carbon, Silicon, Nitrogen, and Phosphorus.

The term “peroxide” as used herein refers to organic compounds containing ROOH or ROOR′ functional groups, which includes hydroperoxides and substituted peroxides. Peroxide values are determined by a titration method and results are reported in ppm (meq/Kg). Untreated olefinic feedstocks may have PV>200 ppm. Olefin metathesis are the most efficient with olefinic feedstock PV<1 ppm. Preferred olefinic feedstock's PV<200 ppm for olefin metathesis reactions. More preferred olefinic feedstock's PV<10 ppm for olefin metathesis reactions. Most preferred olefinic feedstock's PV<1 ppm for olefin metathesis reactions.

The term “catalyst poisons” as used herein refers to compounds that coordinate to or change the metathesis catalyst as to reduce the metathesis catalyst efficiency which results in lower product yields. Examples of catalyst poisons include but not limited to oxygen, water, caustics, amines, carboxylic acids, aldehydes, alcohols, nitriles, phospholipids, tocopherols, d-shingosine, etc.

The term “alpha-olefin” as used herein refers to organic compounds which are terminal olefins or alkenes with a chemical formula RR′C═CH₂, where R and R′ are each independently alkyl, aryl, heteroalkyl, heteroaryl, alkoxy, alkylene, alkenyl, alkenylene, alkynyl, alkynylene, aryloxy alkaryl, or acyl, and R and R′ are not both H.

The term “alkyl” as used herein refers to a linear, branched, or cyclic saturated hydrocarbon group typically although not necessarily containing 1 to about 24 carbon atoms, preferably 1 to about 12 carbon atoms, such as methyl, ethyl, n-propyl, isopropyl, n-butyl, isobutyl, t-butyl, octyl, decyl, and the like, as well as cycloalkyl groups such as cyclopentyl, cyclohexyl and the like. Generally, although again not necessarily, alkyl groups herein contain 1 to about 12 carbon atoms. The term “lower alkyl” intends an alkyl group of 1 to 6 carbon atoms, and the specific term “cycloalkyl” intends a cyclic alkyl group, typically having 4 to 8, preferably 5 to 7, carbon atoms. The term “substituted alkyl” refers to alkyl substituted with one or more substituent groups, and the terms “heteroatom-containing alkyl” and “heteroalkyl” refer to alkyl in which at least one carbon atom is replaced with a heteroatom. If not otherwise indicated, the terms “alkyl” and “lower alkyl” include linear, branched, cyclic, unsubstituted, substituted, and/or heteroatom-containing alkyl and lower alkyl, respectively.

The term “alkylene” as used herein refers to a difunctional linear, branched, or cyclic alkyl group, where “alkyl” is as defined above.

The term “alkenyl” as used herein refers to a linear, branched, or cyclic hydrocarbon group of 2 to about 24 carbon atoms containing at least one double bond, such as ethenyl, n-propenyl, isopropenyl, n-butenyl, isobutenyl, octenyl, decenyl, tetradecenyl, hexadecenyl, eicosenyl, tetracosenyl, and the like. Preferred alkenyl groups herein contain 2 to about 12 carbon atoms. The term “lower alkenyl” intends an alkenyl group of 2 to 6 carbon atoms, and the specific term “cycloalkenyl” intends a cyclic alkenyl group, preferably having 5 to 8 carbon atoms. The term “substituted alkenyl” refers to alkenyl substituted with one or more substituent groups, and the terms “heteroatom-containing alkenyl” and “heteroalkenyl” refer to alkenyl in which at least one carbon atom is replaced with a heteroatom. If not otherwise indicated, the terms “alkenyl” and “lower alkenyl” include linear, branched, cyclic, unsubstituted, substituted, and/or heteroatom-containing alkenyl and lower alkenyl, respectively.

The term “alkenylene” as used herein refers to a difunctional linear, branched, or cyclic alkenyl group, where “alkenyl” is as defined above.

The term “alkynyl” as used herein refers to a linear or branched hydrocarbon group of 2 to about 24 carbon atoms containing at least one triple bond, such as ethynyl, n-propynyl, and the like. Preferred alkynyl groups herein contain 2 to about 12 carbon atoms. The term “lower alkynyl” intends an alkynyl group of 2 to 6 carbon atoms. The term “substituted alkynyl” refers to alkynyl substituted with one or more substituent groups, and the terms “heteroatom-containing alkynyl” and “heteroalkynyl” refer to alkynyl in which at least one carbon atom is replaced with a heteroatom. If not otherwise indicated, the terms “alkynyl” and “lower alkynyl” include linear, branched, unsubstituted, substituted, and/or heteroatom-containing alkynyl and lower alkynyl, respectively.

The term “alkynylene” as used herein refers to a difunctional alkynyl group, where “alkynyl” is as defined above.

The term “alkoxy” as used herein intends an alkyl group bound through a single, terminal ether linkage; that is, an “alkoxy” group may be represented as —O-alkyl where alkyl is as defined above. A “lower alkoxy” group intends an alkoxy group containing 1 to 6 carbon atoms. Analogously, “alkenyloxy” and “lower alkenyloxy” respectively refer to an alkenyl and lower alkenyl group bound through a single, terminal ether linkage, and “alkynyloxy” and “lower alkynyloxy” respectively refer to an alkynyl and lower alkynyl group bound through a single, terminal ether linkage.

The term “aryl” as used herein, and unless otherwise specified, refers to an aromatic substituent containing a single aromatic ring or multiple aromatic rings that are fused together, directly linked, or indirectly linked (such that the different aromatic rings are bound to a common group such as a methylene or ethylene moiety). Preferred aryl groups contain 5 to 24 carbon atoms, and particularly preferred aryl groups contain 5 to 14 carbon atoms. Exemplary aryl groups contain one aromatic ring or two fused or linked aromatic rings, e.g., phenyl, naphthyl, biphenyl, diphenylether, diphenylamine, benzophenone, and the like. “Substituted aryl” refers to an aryl moiety substituted with one or more substituent groups, and the terms “heteroatom containing aryl” and “heteroaryl” refer to aryl substituents in which at least one carbon atom is replaced with a heteroatom, as will be described in further detail infra.

The term “aryloxy” as used herein refers to an aryl group bound through a single, terminal ether linkage, wherein “aryl” is as defined above. An “aryloxy” group may be represented as —O-aryl where aryl is as defined above. Preferred aryloxy groups contain 5 to 24 carbon atoms, and particularly preferred aryloxy groups contain 5 to 14 carbon atoms. Examples of aryloxy groups include, without limitation, phenoxy, o-halo-phenoxy, m-halo-phenoxy, phalo-phenoxy, o-methoxy-phenoxy, m-methoxy-phenoxy, p-methoxy-phenoxy, 2,4-dimethoxyphenoxy, 3,4,5-trimethoxy-phenoxy, and the like.

The term “alkaryl” refers to an aryl group with an alkyl substituent, and the term “aralkyl” refers to an alkyl group with an aryl substituent, wherein “aryl” and “alkyl” are as defined above. Preferred alkaryl and aralkyl groups contain 6 to 24 carbon atoms, and particularly preferred alkaryl and aralkyl groups contain 6 to 16 carbon atoms. Alkaryl groups include, for example, p-methylphenyl, 2,4-dimethylphenyl, p-cyclohexylphenyl, 2,7-dimethylnaphthyl, 7-cyclooctylnaphthyl, 3-ethyl-cyclopenta-1,4-diene, and the like. Examples of aralkyl groups include, without limitation, benzyl, 2-phenyl-ethyl, 3-phenyl-propyl, 4-phenylbutyl, 5-phenyl-pentyl, 4-phenylcyclohexyl, 4-benzylcyclohexyl, 4-phenylcyclohexylmethyl, 4-benzylcyclohexylmethyl, and the like. The terms “alkaryloxy” and “aralkyloxy” refer to substituents of the formula —OR wherein R is alkaryl or aralkyl, respectively, as just defined.

The term “acyl” refers to substituents having the formula —(CO)-alkyl, —(CO)-aryl, or —(CO)-aralkyl, and the term “acyloxy” refers to substituents having the formula —O(CO)-alkyl, —O(CO)-aryl, or —O(CO)-aralkyl, wherein “alkyl,” “aryl,” and “aralkyl” are as defined above.

The terms “cyclic” and “ring” refer to alicyclic or aromatic groups that may or may not be substituted and/or heteroatom-containing, and that may be monocyclic, bicyclic, or polycyclic. The term “alicyclic” is used in the conventional sense to refer to an aliphatic cyclic moiety, as opposed to an aromatic cyclic moiety, and may be monocyclic, bicyclic, or polycyclic.

The terms “halo” and “halogen” are used in the conventional sense to refer to a chloro, bromo, fluoro, or iodo substituent.

“Hydrocarbyl” refers to univalent hydrocarbyl radicals containing 1 to about 30 carbon atoms, preferably 1 to about 24 carbon atoms, most preferably 1 to about 12 carbon atoms, including linear, branched, cyclic, saturated and unsaturated species, such as alkyl groups, alkenyl groups, aryl groups, and the like. The term “lower hydrocarbyl” intends a hydrocarbyl group of 1 to 6 carbon atoms, preferably 1 to 4 carbon atoms, and the term “hydrocarbylene” intends a divalent hydrocarbyl moiety containing 1 to about 30 carbon atoms, preferably 1 to about 24 carbon atoms, most preferably 1 to about 12 carbon atoms, including linear, branched, cyclic, saturated and unsaturated species. The term “lower hydrocarbylene” intends a hydrocarbylene group of 1 to 6 carbon atoms. “Substituted hydrocarbyl” refers to hydrocarbyl substituted with one or more substituent groups, and the terms “heteroatom-containing hydrocarbyl” and “heterohydrocarbyl” refer to hydrocarbyl in which at least one carbon atom is replaced with a heteroatom. Similarly, “substituted hydrocarbylene” refers to hydrocarbylene substituted with one or more substituent groups, and the terms “heteroatom-containing hydrocarbylene” and “heterohydrocarbylene” refer to hydrocarbylene in which at least one carbon atom is replaced with a heteroatom. Unless otherwise indicated, the term “hydrocarbyl” and “hydrocarbylene” are to be interpreted as including substituted and/or heteroatom-containing hydrocarbyl and hydrocarbylene moieties, respectively.

The term “heteroatom-containing” as in a “heteroatom-containing hydrocarbyl group” refers to a hydrocarbon molecule or a hydrocarbyl molecular fragment in which one or more carbon atoms is replaced with an atom other than carbon, e.g., nitrogen, oxygen, sulfur, phosphorus, or silicon, typically nitrogen, oxygen, or sulfur. Similarly, the term “heteroalkyl” refers to an alkyl substituent that is heteroatom-containing, the term “heterocyclic” refers to a cyclic substituent that is heteroatom-containing, the terms “heteroaryl” and “heteroaromatic” respectively refer to “aryl” and “aromatic” substituents that are heteroatom-containing, and the like. It should be noted that a “heterocyclic” group or compound may or may not be aromatic, and further that “heterocycles” may be monocyclic, bicyclic, or polycyclic as described above with respect to the term “aryl.” Examples of heteroalkyl groups include alkoxyaryl, alkylsulfanyl-substituted alkyl, N-alkylated amino alkyl, and the like. Examples of heteroaryl substituents include pyrrolyl, pyrrolidinyl, pyridinyl, quinolinyl, indolyl, pyrimidinyl, imidazolyl, 1,2,4-triazolyl, tetrazolyl, etc., and examples of heteroatom-containing alicyclic groups are pyrrolidino, morpholino, piperazino, piperidino, etc.

The term “heterocyclic carbene” refers to a neutral electron donor ligand comprising a carbene molecule, where the carbenic carbon atom is contained within a cyclic structure and where the cyclic structure also contains at least one heteroatom. Examples of heterocyclic carbenes include “N-heterocyclic carbenes” wherein the heteroatom is nitrogen and “P-heterocyclic carbenes” wherein the heteroatom is phosphorus.

By “substituted” as in “substituted hydrocarbyl,” “substituted alkyl,” “substituted aryl,” and the like, as alluded to in some of the aforementioned definitions, is meant that in the hydrocarbyl, alkyl, aryl, or other moiety, at least one hydrogen atom bound to a carbon (or other) atom is replaced with one or more non-hydrogen substituents. Examples of such substituents include, without limitation: functional groups referred to herein as “Fn,” such as halo, hydroxyl, sulfhydryl, C₁-C₂₄ alkoxy, C₂-C₂₄ alkenyloxy, C₂-C₂₄ alkynyloxy, C₅-C₂₄ aryloxy, C₆-C₂₄ aralkyloxy, C₆-C₂₄ alkaryloxy, acyl (including C₂-C₂₄ alkylcarbonyl (—CO-alkyl) and C₆-C₂₄ arylcarbonyl (—CO-aryl)), acyloxy (—O-acyl, including C₂-C₂₄ alkylcarbonyloxy (—O—CO-alkyl) and C₆-C₂₄ arylcarbonyloxy (—O—CO-aryl)), C₂-C₂₄ alkoxycarbonyl (—(CO)—O-alkyl), C₆-C₂₄ aryloxycarbonyl (—(CO)—O-aryl), halocarbonyl (—CO)—X where X is halo), C₂-C₂₄ alkylcarbonato (—O—(CO)—O-alkyl), C₆-C₂₄ arylcarbonato (—O—(CO)—O-aryl), carboxy (—COOH), carboxylate (—COO—), carbamoyl (—(CO)—NH₂), mono-(C₁-C₂₄ alkyl)-substituted carbamoyl ((CO)—NH(C₁-C₂₄ alkyl)), di-(C₁-C₂₄ alkyl)-substituted carbamoyl ((CO)—N(C₁-C₂₄ alkyl)₂), mono-(C₅-C₂₄ aryl)-substituted carbamoyl (—(CO)—NH-aryl), di-(C₅-C₂₄ aryl)-substituted carbamoyl (—(CO)—N(C₅-C₂₄ aryl)₂), di-N—(C₁-C₂₄ alkyl), N—(C₅-C₂₄ aryl)-substituted carbamoyl, thiocarbamoyl (—(CS)—NH₂), mono-(C₁-C₂₄ alkyl)-substituted thiocarbamoyl (—(CO)—NH(C₁-C₂₄ alkyl)), di-(C₁-C₂₄ alkyl)-substituted thiocarbamoyl (—(CO)—N(C₁-C₂₄ alkyl)₂), mono-(C₅-C₂₄ aryl)-substituted thiocarbamoyl (—(CO)—NH-aryl), di-(C₅-C₂₄ aryl)-substituted thiocarbamoyl (—(CO)—N(C₅-C₂₄ aryl)₂), di-N—(C₁-C₂₄ alkyl), N—(C₅-C₂₄ aryl)-substituted thiocarbamoyl, carbamido (—NH—(CO)—NH₂), cyano (—C≡N), cyanato (—O—C≡N), thiocyanato (—S—C≡N), formyl (—(CO)—H), thioformyl (—(CS)—H), amino (—NH₂), mono-(C₁-C₂₄ alkyl)-substituted amino, di-(C₁-C₂₄ alkyl)-substituted amino, mono-(C₅-C₂₄ aryl)-substituted amino, di-(C₅-C₂₄ aryl)-substituted amino, C₂-C₂₄ alkylamido (—NH—(CO)-alkyl), C₆-C₂₄ arylamido (—NH—(CO)-aryl), imino (—CR═NH where R=hydrogen, C₁-C₂₄ alkyl, C₅-C₂₄ aryl, C₆-C₂₄ alkaryl, C₆-C₂₄ aralkyl, etc.), C₂-C₂₀ alkylimino (—CR═N(alkyl), where R=hydrogen, C₁-C₂₄ alkyl, C₅-C₂₄ aryl, C₆-C₂₄ alkaryl, C₆-C₂₄ aralkyl, etc.), arylimino (—CR═N(aryl), where R=hydrogen, C₁-C₂₀ alkyl, C₅-C₂₄ aryl, C₆-C₂₄ alkaryl, C₆-C₂₄ aralkyl, etc.), nitro (—NO₂), nitroso (—NO), sulfo (—SO₂—OH), sulfonato (—SO₂—O—), C₁-C₂₄ alkylsulfanyl (—S-alkyl; also termed “alkylthio”), C₅-C₂₄ arylsulfanyl (—S-aryl; also termed “arylthio”), C₁-C₂₄ alkylsulfinyl (—(SO)-alkyl), C₅-C₂₄ arylsulfinyl (—(SO)-aryl), C₁-C₂₄ alkylsulfonyl (—SO₂-alkyl), C₅-C₂₄ arylsulfonyl (—SO₂-aryl), boryl (—BH₂), borono (—B(OH)₂), boronato (—B(OR)₂ where R is alkyl or other hydrocarbyl), phosphono (—P(O)(OH)₂), phosphonato (—P(O)(O—)₂), phosphinato (—P(O)(O—)), phospho (—PO₂), phosphino (—PH₂), silyl (—SiR₃ wherein R is hydrogen or hydrocarbyl), and silyloxy (—O-silyl); and the hydrocarbyl moieties C₁-C₂₄ alkyl (preferably C₁-C₁₂ alkyl, more preferably C₁-C₆ alkyl), C₂-C₂₄ alkenyl (preferably C₂-C₁₂ alkenyl, more preferably C₂-C₆ alkenyl), C₂-C₂₄ alkynyl (preferably C₂-C₁₂ alkynyl, more preferably C₂-C₆ alkynyl), C₅-C₂₄ aryl (preferably C₅-C₁₄ aryl), C₆-C₂₄ alkaryl (preferably C₆-C₁₆ alkaryl), and C₆-C₂₄ aralkyl (preferably C₆-C₁₆ aralkyl).

In addition, the aforementioned functional groups may, if a particular group permits, be further substituted with one or more additional functional groups or with one or more hydrocarbyl moieties such as those specifically enumerated above. Analogously, the above mentioned hydrocarbyl moieties may be further substituted with one or more functional groups or additional hydrocarbyl moieties such as those specifically enumerated.

“Optional” or “optionally” means that the subsequently described circumstance may or may not occur, so that the description includes instances where the circumstance occurs and instances where it does not. For example, the phrase “optionally substituted” means that a non-hydrogen substituent may or may not be present on a given atom, and, thus, the description includes structures wherein a non-hydrogen substituent is present and structures wherein a non-hydrogen substituent is not present.

In the molecular structures herein, the use of bold and dashed lines to denote particular conformation of groups follows the IUPAC convention. A bond indicated by a broken line indicates that the group in question is below the general plane of the molecule as drawn, and a bond indicated by a bold line indicates that the group at the position in question is above the general plane of the molecule as drawn.

### Methods Compositions and Reaction Systems

Accordingly, herein is described an olefin cross-metathesis method for synthesizing a terminal olefin from an olefinic substrate comprised of at least one internal olefin and a cross metathesis partner comprised of an alpha olefinic reactant. The reaction is carried out catalytically, in the presence of a ruthenium alkylidene metathesis catalyst.

The olefinic substrate comprises at least one internal olefin, and may have 2 or more internal olefins. For example, the olefinic substrate may comprise in the range of 2 to about 15, 2 to about 10, or 2 to about 5 internal olefins. By “internal olefin” is meant an olefin wherein each of the olefinic carbons is substituted by at least one non-hydrogen substituent. The non-hydrogen substituents are selected from hydrocarbyl, substituted hydrocarbyl, heteroatom-containing hydrocarbyl, substituted heteroatom-containing hydrocarbyl, and functional groups. The internal olefin is therefore at least disubstituted, and may further include additional non-hydrogen substituents such that the internal olefin is tri- or tetra-substituted. Each of the substituents on the internal olefinic carbons may be further substituted as described supra. The internal olefin may be in the Z- or E-configuration. When the olefinic substrate comprises a plurality of internal olefins, the olefinic substrate may comprise a mixture of internal olefins (varying in stereochemistry and/or substituent identity), or may comprise a plurality of internal olefins.

The olefinic substrate may be a single compound or a mixture of compounds. The olefinic substrate may be hydrophobic or hydrophilic, although in a preferred embodiment, the olefinic substrate is hydrophobic.

For example, the olefinic substrate may be represented by the formula (R^{I})(R^{II})C═C(R^{III})(R^{IV}), wherein R^{I}, R^{II}, R^{III}, and R^{IV} are independently selected from hydrogen, hydrocarbyl, substituted hydrocarbyl, heteroatom-containing hydrocarbyl, substituted heteroatom-containing hydrocarbyl, and functional groups, provided that at least one of R^{I} and R^{II} and at least one of R^{III} and R^{IV} is other than hydrogen. In a preferred embodiment, either R^{I} or R^{II} and either R^{III} or R^{IV} is hydrogen, such that the internal olefin is di-substituted.

As another example, the olefinic substrate is a natural seed oil containing an ester of glycerol (a “glyceride”), and has the structure of formula (I)

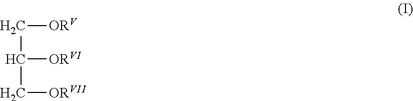

wherein R^{V}, R^{VI}, and R^{VII} are independently selected from hydrogen, hydrocarbyl, substituted hydrocarbyl, heteroatom-containing hydrocarbyl, substituted heteroatom-containing hydrocarbyl, and functional groups, provided that at least one of R^{V}, R^{VI}, and R^{VII} is other than hydrogen and comprises an internal olefin. In a preferred embodiment, the olefinic substrate comprises glycerol esterified with 1, 2, or 3 fatty acids, such that the olefinic substrate is a monoacylglycerol, diacylglycerol, or triacylglycerol (i.e., a monoglyceride, diglyceride, or triglyceride, respectively), or a mixture thereof. Each fatty acid-derived fragment of the olefinic substrate may independently be saturated, monounsaturated, or polyunsaturated, and may furthermore derive (or be derivable) from naturally-occurring fatty acids or from synthetic fatty acids. For example, the olefinic substrate may comprise glycerol esterified with one, two, or three fatty acids that are independently selected from CH₃(CH₂)—COOH, where n is 2, 3, 4, 5, 6, 7, 8, 9, 10, 11, 12, 13, 14, 15, 16, 17, 18, 19, 20, 21, or 22, palmitoleic acid, vaccenic acid, erucic acid, oleic acid, alpha-linolenic acid, gamma-linolenic acid, linoleic acid, gadoleic acid, arachidonic acid, docosahexaenoic acid (i.e., DHA), eicosapentaenoic acid (i.e., EPA), and CH₃—R^{VIII}—COOH, where R^{VIII} is substituted or unsubstituted C₂-C₂₄ alkenylene. The olefinic substrate may be solid (e.g., a fat) or liquid (e.g., an oil).

Preferred natural seed oils containing glycerides that may be used as the olefinic substrate are seed oils, or are compounds that derive from seed oils. Preferred seed oil sources include soybean oil, camelina oil, sunflower oil, canola oil, safflower oil, cottonseed oil, castor oil, rapeseed oil, peanut oil, corn oil, olive oil, palm oil, sesame oil, and grape seed oil.

The olefinic substrate may be a compound or mixture of compounds that is derived from natural seed oils containing a glyceride using any one or combination of methods well known in the chemical arts. Such methods include saponification, esterification, hydrogenation, isomerization, oxidation, and reduction. For example, the olefinic substrate may be the carboxylic acid or mixture of carboxylic acids that result from the saponification of a monoacylglycerol, diacylglycerol, triacylglycerol, or mixture thereof. In a preferred embodiment, the olefinic substrate is a fatty acid methyl ester (FAME), i.e., the methyl ester of a carboxylic acid that is derived from a glyceride. Sunflower FAME, safflower FAME, soy FAME (i.e., methyl soyate), and canola FAME are examples of such olefinic substrates.

The olefinic substrate may contain one or more soluble metal salts at a sufficient concentration to yield a PV<10, but preferably PV<1, and will not inhibit the olefin metathesis catalyst.

In addition, in some embodiments the olefinic substrates include seed oil-derived compounds such as methyl oleate, containing a soluble metal salt.

The cross-metathesis partner that is reacted with the at least one internal olefin may be any olefinic compound that is capable of undergoing a metathesis reaction with the olefinic substrate to generate a terminal alkene product. The cross-metathesis partner comprises an alpha-olefin, wherein one olefinic carbon is unsubstituted and the other olefinic carbon is substituted with one or two non-hydrogen substituents. The substituted olefinic carbon may therefore be mono-substituted or di-substituted. The cross-metathesis partner may comprise a plurality of alpha-olefins. A mixture of alpha-olefins may be used.

The cross-metathesis partner may comprise substituents selected from any of the substituents listed herein above. For example, the cross-metathesis partner may be an alpha-olefin that comprises a substituent comprising 1 to about 20 carbon atoms, about 10 carbon atoms, about 6 carbon atoms, or about 3 carbon atoms.

As an example, the cross-metathesis partner may have the structure H₂C═C(R^{IX})(R^{X}), wherein R^{IX} and R^{X} are independently selected from hydrogen, hydrocarbyl, substituted hydrocarbyl, heteroatom-containing hydrocarbyl, substituted heteroatom-containing hydrocarbyl and functional groups, provided that at least one of R^{IX} and R^{X} is a non-hydrogen substituent. Furthermore, R^{IX} and R^{X} may be linked to form a cycle. In a preferred embodiment, R^{IX} and R^{X} are independently selected from substituted or unsubstituted C₁-C₂₀ alkyl, substituted or unsubstituted C₂-C₂₀ alkenyl, substituted or unsubstituted C₂-C₂₀ alkynyl, substituted or unsubstituted heteroatom-containing C₁-C₂₀ alkyl, substituted or unsubstituted heteroatom-containing C₂-C₂₀ alkenyl, substituted or unsubstituted heteroatom-containing C₂-C₂₀ alkynyl, substituted or unsubstituted C₅-C₂₄ aryl, substituted or unsubstituted C₅-C₂₄ alkaryl, or substituted or unsubstituted C₅-C₂₄ aralkyl, substituted or unsubstituted heteroatom-containing C₅-C₂₄ aryl, substituted or unsubstituted heteroatom-containing C₅-C₂₄ alkaryl, substituted or unsubstituted heteroatom-containing C₅-C₂₄ aralkyl, and functional groups, with the proviso that when R^{X} equals R^{X} R^{IX} and R^{X} are not equal hydrogen.

Examples of monosubstituted alpha-olefins that may be used for the cross-metathesis partner include 1-propene, 1-butene, 1-pentene, 1-hexene, 1-heptene, 1-octene, 1-nonene, 1-decene, 1-undecene, 1-dodecene, 1-tridecene, 1-tetradecene, 1-pentadecene, 1-hexadecene, 1-heptadecene, 1-octadecene, 1-nonadecene, 1-eicosene and larger alpha olefins, 2-propenol, 3-butenol, 4-pentenol, 5-hexenol, 6-heptenol, 7-octenol, 8-nonenol, 9-decenol, 10-undecenol, 11-dodecenol, 12-tridecenol, 13-tetradecenol, 14-pentadecenol, 15-hexadecenol, 16-heptadecenol, 17-octadecenol, 18-nonadecenol, 19-eicosenol and larger alpha alkenols, 2-propenyl acetate, 3-butenyl acetate, 4-pentenyl acetate, 5-hexenyl acetate, 6-heptenyl acetate, 7-octenyl acetate, 8-nonenyl acetate, 9-decenyl acetate, 10-undecenyl acetate, 11-dodecenyl acetate, 12-tridecenyl acetate 13-tetradecenyl acetate, 14-pentadecenyl acetate, 15-hexadecenyl acetate, 16-heptadecenyl acetate, 17-octadecenyl acetate, 18-nonadecenyl acetate, 19-eicosenyl acetate and larger alpha-alkenyl acetates, 2-propenyl chloride, 3-butenyl chloride, 4-pentenyl chloride, 5-hexenyl chloride, 6-heptenyl chloride, 7-octenyl chloride, 8-nonenyl chloride, 9-decenyl chloride, 10-undecenyl chloride, 11-dodecenyl chloride, 12-tridecenyl chloride, 13-tetradecenyl chloride, 14-pentadecenyl chloride, 15-hexadecenyl chloride, 16-heptadecenyl chloride, 17-octadecenyl chloride, 18-nonadecenyl chloride, 19-eicosenyl chloride and larger alpha-alkenyl chlorides, bromides, and iodides, allyl cyclohexane, allyl cyclopentane, and the like.

Examples of disubstituted alpha-olefins that may be used for the cross-metathesis partner include isobutylene, 2-methylbut-1-ene, 2-methylpent-1-ene, 2-methylhex-1-ene, 2-methylhept-1-ene, 2-methyloct-1-ene, and the like.

Any combination of any of the above mentioned alpha olefin and cross metathesis partners can be reacted according to the disclosed methods, compositions, and reaction systems. In an exemplary embodiment, a composition comprising 9-decenoic acid (9-DA) and 9-undecenoic acid (9-UDA) can be prepared by the cross-metathesis of 1-propene with an internal olefin comprising a fatty acid, fatty ester, or mixture thereof. The internal olefin has a carbon-carbon double bond located at the C₉-C₁₀ position in the main chain of the fatty acid or fatty ester. As an example, the internal olefin may have the structure:

CH₃(CH₂)ₙCH═CH(CH₂)₇COOR

where n is an integer (typically 7); and R is hydrogen (fatty acid) or a hydrocarbyl group (fatty ester).

Representative examples of suitable internal olefins include oleic acid, methyl oleate, and mixtures thereof. When a fatty ester is used as the internal olefin, the resulting cross-metathesis products are hydrolyzed according to known techniques in order to convert the ester functional groups into carboxylic acid groups. As is dictated by stoichiometry of the cross-metathesis reaction, the product composition typically comprises about 50 mole % 9-DA and about 50 mole % 9-UDA.

The reactions described herein include as reactants an olefinic substrate and a cross-metathesis partner. Individually, any of the reactants may be solid, liquid, or gaseous, although in a preferred embodiment, the reaction can be carried out under conditions to ensure that the olefinic substrate and the cross-metathesis partner are liquid. The use of a liquid cross-metathesis partner instead of a gaseous cross-metathesis partner, such as ethylene, is advantageous as it allows a convenient controlling of reaction pressures. In addition, in those embodiments, the demand on vapor condensers and vapor reclaiming equipment is reduced or eliminated.

It will be appreciated by those of skill in the art that the use of alpha-olefin cross-metathesis partners containing, for example, long alkyl substituents enables liquid-phase, room temperature (or greater) reactions and/or the use of reactors working at near atmospheric or slightly higher pressures.

In some preferred embodiments, the cross-metathesis partner is soluble in the olefinic substrate. The cross-metathesis partner may have a solubility of at least 0.25 M, at least 1 M, at least 3 M, or at least 5 M in the olefinic substrate, containing a soluble metal salt. The cross-metathesis partner and the olefinic substrate may also be miscible at all concentrations.

As another example, the cross-metathesis partner has a low solubility in the olefinic substrate, and the cross-metathesis reaction occurs as an interfacial reaction. It should be noted that, when one or more of the reactants is solid or gaseous, the reactions may still be carried out in the liquid phase by dissolving any solid or gaseous reactants in the liquid reactants, or by employing a solvent, as described infra.

The cross-metathesis partner may be provided in the form of a gas. Typically, the pressure of a gaseous cross-metathesis partner over the reaction solution is maintained in a range that has a minimum of about 10 psig, 15 psig, 50 psig, or 80 psig, and a maximum of about 250 psig, 200 psig, 150 psig, or 130 psig. Embodiments wherein the reaction pressures are lowered till near atmospheric pressure and in particular till pressures slightly above atmospheric allow for a reduction in equipment costs compared to embodiments performed at high pressure (e.g., pressures greater than 250 psi).

The reactions of the disclosure are catalyzed by any of the metathesis catalysts that are described infra. The catalyst is typically added to the reaction medium as a solid, but may also be added as a solution wherein the catalyst is dissolved in an appropriate solvent. It will be appreciated that the amount of catalyst that is used (i.e., the “catalyst loading”) in the reaction is dependent upon a variety of factors such as the identity of the reactants and the reaction conditions that are employed. It is therefore understood that catalyst loading may be optimally and independently chosen for each reaction. In general, however, the catalyst will be present in an amount that ranges from a low of about 0.1 ppm, 1 ppm, or 5 ppm, to a high of about 10 ppm, 15 ppm, 25 ppm, 50 ppm, 100 ppm, 200 ppm, 500 ppm, or 1000 ppm relative to the amount of the olefinic substrate. Catalyst loading, when measured in ppm relative to the amount of the olefinic substrate, is calculated using the equation

$$ppm catalyst = \frac{moles catalyst}{\begin{matrix}moles olefinic substrate \\ double bonds\end{matrix}} * 1 , 000 , 000$$

Alternatively, the amount of catalyst can be measured in terms of mol % relative to the amount of olefinic substrate, using the equation

$$mol \% catalyst = \frac{moles catalyst}{moles olefinic substrate double bonds } * 100.$$

Thus, the catalyst will generally be present in an amount that ranges from a low of about 0.00001 mol %, 0.0001 mol %, or 0.0005 mol %, to a high of about 0.001 mol %, 0.0015 mol %, 0.0025 mol %, 0.005 mol %, 0.01 mol %, 0.02 mol %, 0.05 mol %, or 0.1 mol % relative to the olefinic substrate.

In a preferred embodiment, the reactions of the disclosure are carried out under a dry, inert atmosphere. Such an atmosphere may be created using any inert gas, including such gases as nitrogen and argon. The use of an inert atmosphere is optimal in terms of promoting catalyst activity, and reactions performed under an inert atmosphere typically are performed with relatively low catalyst loading. The reactions of the disclosure may also be carried out in an oxygen-containing and/or a water-containing atmosphere, and in one embodiment, the reactions are carried out under ambient conditions. The presence of oxygen, water, or other impurities in the reaction may, however, necessitate the use of higher catalyst loadings as compared with reactions performed under an inert atmosphere.

Any soluble metal salts, in an olefin metathesis substrate, which reduces the peroxide level or otherwise improve the activity of the metathesis reaction are suitable for the invention. Suitable soluble metal salts include aluminum isopropoxide (Al(OⁱPr)₃), magnesium aluminum isopropoxide (MgAl₂(OⁱPr)₈), titanium (IV) isopropoxide (Ti(OⁱPr)₄), and methylaluminoxane (MAO). Other non-limiting examples of soluble salts include bismuth neodecanoate (Bi(Neodec)₃), and cerium acetate hydrate (Ce(OAc)₃ xH₂O). The soluble metal salt is usually mixed with the olefin and stirred from 2 to 96 hours. The mixture may be heated slightly above room temperature, i.e., 30° C., or up to 80° C. The loading of the soluble metal salt may be from 0.1 wt % to 5 wt %, with preferred loadings of 0.5 wt % to 2.0 wt %. The soluble metal salt may be added directly to the olefin or as a solution in another solvent such as toluene.

### Olefin Metathesis Catalysts

The invention is useful for the purification of olefins for any suitable metathesis reaction. Such metathesis reactions are not specifically limited, and include ring-closing metathesis (RCM), ring-opening metathesis polymerization (ROMP), cross metathesis (CM), ring-opening cross metathesis (ROCM), self-metathesis, ethenolysis, alkenolysis, acyclic diene metathesis polymerization, and combinations thereof.

The olefin metathesis catalyst complex according to the invention is preferably a Group 8 transition metal complex having the structure of formula (I)

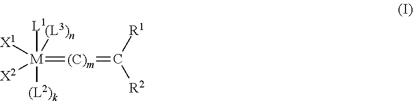

in which:

M is a Group 8 transition metal;

L¹, L², and L³ are neutral electron donor ligands;

n is 0 or 1, such that L³ may or may not be present;

m is 0, 1, or 2;

k is 0 or 1;

X¹ and X² are anionic ligands; and

R¹ and R² are independently selected from hydrogen, hydrocarbyl, substituted hydrocarbyl, heteroatom-containing hydrocarbyl, substituted heteroatom-containing hydrocarbyl, and functional groups, wherein any two or more of X¹, X², L¹, L², L³, R¹, and R² can be taken together to form one or more cyclic groups, and further wherein any one or more of X¹, X², L¹, L², L³, R¹, and R² may be attached to a support.

Additionally, in formula (I), one or both of R¹ and R² may have the structure —(W)ₙ—U⁺V⁻, in which W is selected from hydrocarbylene, substituted hydrocarbylene, heteroatom-containing hydrocarbylene, or substituted heteroatom-containing hydrocarbylene; U is a positively charged Group 15 or Group 16 element substituted with hydrogen, hydrocarbyl, substituted hydrocarbyl, heteroatom-containing hydrocarbyl, or substituted heteroatom-containing hydrocarbyl; V is a negatively charged counterion; and n is zero or 1. Furthermore, R¹ and R² may be taken together to form an indenylidene moiety.

Preferred catalysts contain Ru or Os as the Group 8 transition metal, with Ru particularly preferred.

Numerous embodiments of the catalysts useful in the reactions disclosed herein are described in more detail infra. For the sake of convenience, the catalysts are described in groups, but it should be emphasized that these groups are not meant to be limiting in any way. That is, any of the catalysts useful in the invention may fit the description of more than one of the groups described herein.

A first group of catalysts, then, are commonly referred to as First Generation Grubbs-type catalysts, and have the structure of formula (I). For the first group of catalysts, M is a Group 8 transition metal, m is 0, 1, or 2, and n, X¹, X², L¹, L², L³, and R² are described as follows.

For the first group of catalysts, n is 0, and L¹ and L² are independently selected from phosphine, sulfonated phosphine, phosphite, phosphinite, phosphonite, arsine, stibine, ether, (including cyclic ethers), amine, amide, imine, sulfoxide, carboxyl, nitrosyl, pyridine, substituted pyridine, imidazole, substituted imidazole, pyrazine, substituted pyrazine and thioether. Exemplary ligands are trisubstituted phosphines. Preferred trisubstituted phosphines are of the formula PR^{H1}R^{H2}R^{H3}, where R^{H1}, R^{H2}, and R^{H3} are each independently substituted or unsubstituted aryl or C₁-C₁₀ alkyl, particularly primary alkyl, secondary alkyl, or cycloalkyl. In the most preferred, L¹ and L² are independently selected from the group consisting of trimethylphosphine (PMe₃), triethylphosphine (PEt₃), tri-n-butylphosphine (PBu₃), tri(ortho-tolyl)phosphine (P-o-tolyl₃), tri-tert-butylphosphine (P-tert-Bu₃), tricyclopentylphosphine (PCyclopentyl₃), tricyclohexylphosphine (PCy₃), triisopropylphosphine (P-i-Pr₃), trioctylphosphine (POct₃), triisobutylphosphine, (P-i-Bu₃), triphenylphosphine (PPh₃), tri(pentafluorophenyl)phosphine (P(C₆F₅)₃), methyldiphenylphosphine (PMePh₂), dimethylphenylphosphine (PMe₂Ph), and diethylphenylphosphine (PEt₂Ph).

Alternatively, L¹ and L² may be independently selected from phosphabicycloalkane (e.g., monosubstituted 9-phosphabicyclo-[3.3.1]nonane, or monosubstituted 9-phosphabicyclo[4.2.1]nonane] such as cyclohexylphoban, isopropylphoban, ethylphoban, methylphoban, butylphoban, pentylphoban, and the like).

X¹ and X² are anionic ligands, and may be the same or different, or are linked together to form a cyclic group, typically although not necessarily a five- to eight-membered ring. In preferred embodiments, X¹ and X² are each independently hydrogen, halide, or one of the following groups: C₁-C₂₀ alkyl, C₅-C₂₄ aryl, C₁-C₂₀ alkoxy, C₅-C₂₄ aryloxy, C₂-C₂₀ alkoxycarbonyl, C₆-C₂₄ aryloxycarbonyl, C₂-C₂₄ acyl, C₂-C₂₄ acyloxy, C₁-C₂₀ alkylsulfonato, C₅-C₂₄ arylsulfonato, C₁-C₂₀ alkylsulfanyl, C₅-C₂₄ arylsulfanyl, C₁-C₂₀ alkylsulfinyl, NO₃, —N═C═O, —N═C═S, or C₅-C₂₄ arylsulfinyl. Optionally, X¹ and X² may be substituted with one or more moieties selected from C₁-C₁₂ alkyl, C₁-C₁₂ alkoxy, C₅-C₂₄ aryl, and halide, which may, in turn, with the exception of halide, be further substituted with one or more groups selected from halide, C₁-C₆ alkyl, C₁-C₆ alkoxy, and phenyl. In more preferred embodiments, X¹ and X² are each a halide, benzoate, C₂-C₆ acyl, C₂-C₆ alkoxycarbonyl, C₁-C₆ alkyl, phenoxy, C₁-C₆ alkoxy, C₁-C₆ alkylsulfanyl, aryl, or C₁-C₆ alkylsulfonyl. In even more preferred embodiments, X¹ and X² are each halide, CF₃CO₂, CH₃CO₂, CFH₂CO₂, (CH₃)₃CO, (CF₃)₂(CH₃)CO, (CF₃)(CH₃)₂CO, PhO, MeO, EtO, tosylate, mesylate, or trifluoromethane-sulfonate. In the most preferred embodiments, X¹ and X² are each chloride.

R¹ and R² are independently selected from hydrogen, hydrocarbyl (e.g., C₁-C₂₀ alkyl, C₂-C₂₀ alkenyl, C₂-C₂₀ alkynyl, C₅-C₂₄ aryl, C₆-C₂₄ alkaryl, C₆-C₂₄ aralkyl, etc.), substituted hydrocarbyl (e.g., substituted C₁-C₂₀ alkyl, C₂-C₂₀ alkenyl, C₂-C₂₀ alkynyl, C₅-C₂₄ aryl, C₆-C₂₄ alkaryl, C₆-C₂₄ aralkyl, etc.), heteroatom-containing hydrocarbyl (e.g., heteroatom-containing C₁-C₂₀ alkyl, C₂-C₂₀ alkenyl, C₂-C₂₀ alkynyl, C₅-C₂₄ aryl, C₆-C₂₄ alkaryl, C₆-C₂₄ aralkyl, etc.), and substituted heteroatom-containing hydrocarbyl (e.g., substituted heteroatom-containing C₁-C₂₀ alkyl, C₂-C₂₀ alkenyl, C₂-C₂₀ alkynyl, C₅-C₂₄ aryl, C₆-C₂₄ alkaryl, C₆-C₂₄ aralkyl, etc.), and functional groups. R¹ and R² may also be linked to form a cyclic group, which may be aliphatic or aromatic, and may contain substituents and/or heteroatoms. Generally, such a cyclic group will contain 4 to 12, preferably 5, 6, 7, or 8 ring atoms.

In preferred catalysts, R¹ is hydrogen and R² is selected from C₁-C₂₀ alkyl, C₂-C₂₀ alkenyl, and C₅-C₂₄ aryl, more preferably C₁-C₆ alkyl, C₂-C₆ alkenyl, and C₅-C₁₄ aryl. Still more preferably, R² is phenyl, vinyl, methyl, isopropyl, or t-butyl, optionally substituted with one or more moieties selected from C₁-C₆ alkyl, C₁-C₆ alkoxy, phenyl, and a functional group Fn as defined earlier herein. Most preferably, R² is phenyl or vinyl substituted with one or more moieties selected from methyl, ethyl, chloro, bromo, iodo, fluoro, nitro, dimethylamino, methyl, methoxy, and phenyl. Optimally, R² is phenyl or —CH═C(CH₃)₂.

Any two or more (typically two, three, or four) of X¹, X², L¹, L², L³, R¹, and R² can be taken together to form a cyclic group, including bidentate or multidentate ligands, as disclosed, for example, in U.S. Pat. No. 5,312,940, the disclosure of which is incorporated herein by reference. When any of X¹, X², L¹, L², L³, R¹, and R² are linked to form cyclic groups, those cyclic groups may contain 4 to 12, preferably 4, 5, 6, 7, or 8 atoms, or may comprise two or three of such rings, which may be either fused or linked. The cyclic groups may be aliphatic or aromatic, and may be heteroatom-containing and/or substituted. The cyclic group may, in some cases, form a bidentate ligand or a tridentate ligand. Examples of bidentate ligands include, but are not limited to, bisphosphines, dialkoxides, alkyldiketonates, and aryldiketonates.

A second group of catalysts, commonly referred to as Second Generation Grubbs-type catalysts, have the structure of formula (I), wherein L¹ is a carbene ligand having the structure of formula (II)

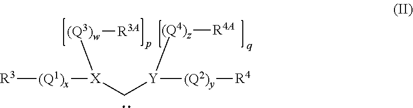

such that the complex may have the structure of formula (III)

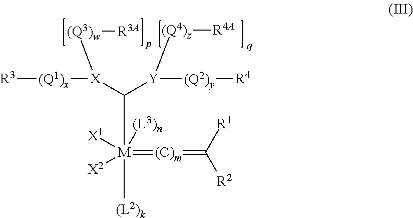

wherein M, m, n, X¹, X², L², L³, R¹, and R² are as defined for the first group of catalysts, and the remaining substituents are as follows;

X and Y are heteroatoms typically selected from N, O, S, and P. Since O and S are divalent, p is necessarily zero when X is O or S, q is necessarily zero when Y is O or S, and k is zero or 1. However, when X is N or P, then p is 1, and when Y is N or P, then q is 1. In a preferred embodiment, both X and Y are N;

Q¹, Q², Q³, and Q⁴ are linkers, e.g., hydrocarbylene (including substituted hydrocarbylene, heteroatom-containing hydrocarbylene, and substituted heteroatom-containing hydrocarbylene, such as substituted and/or heteroatom-containing alkylene) or —(CO)—, and w, x, y, and z are independently zero or 1, meaning that each linker is optional. Preferably, w, x, y, and z are all zero. Further, two or more substituents on adjacent atoms within Q¹ and Q⁴ may Q¹, Q², Q³, be linked to form an additional cyclic group; and R³, R^{3A}, R⁴, and R^{4A} are independently selected from hydrogen, hydrocarbyl, substituted hydrocarbyl, heteroatom-containing hydrocarbyl, and substituted heteroatom-containing hydrocarbyl. In addition, X and Y may be independently selected from carbon and one of the heteroatoms mentioned above. Also, L² and L³ may be taken together to form a single bidentate electron-donating heterocyclic ligand. Furthermore, R¹ and R² may be taken together to form an indenylidene moiety. Moreover, X¹, X², L², L³, X, and Y may be further coordinated to boron or to a carboxylate.

In addition, any two or more of X¹, X², L¹, L², L³, R¹, R², R³, R^{3A}, R⁴, R^{4A}, Q¹, Q², Q³, and Q⁴ can be taken together to form a cyclic group, and any one or more of X¹, X², L², L³, Q¹, Q², Q³, Q⁴, R¹, R², R³, R^{3A}, R⁴, and R^{4A} may be attached to a support. Any two or more of X¹, X², L¹, L², L³, R¹, R², R³, R^{3A}, R⁴, and R^{4A} can also be taken to be -A-Fn, wherein “A” is a divalent hydrocarbon moiety selected from alkylene and arylalkylene, wherein the alkyl portion of the alkylene and arylalkylene groups can be linear or branched, saturated or unsaturated, cyclic or acyclic, and substituted or unsubstituted, wherein the aryl portion of the of arylalkylene can be substituted or unsubstituted, and wherein hetero atoms and/or functional groups may be present in either the aryl or the alkyl portions of the alkylene and arylalkylene groups, and Fn is a functional group, or together to form a cyclic group, and any one or more of X¹, X², L², L³, Q¹, Q², Q³, Q⁴, R¹, R², R³, R^{3A}, R⁴, and R^{4A} may be attached to a support.

Preferably, R^{3A} and R^{4A} are linked to form a cyclic group so that the carbene ligand has the structure of formula (IV)

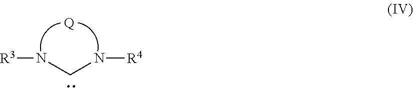

wherein R³ and R⁴ are as defined for the second group of catalysts above, with preferably at least one of R³ and R⁴, and more preferably both R³ and R⁴, being alicyclic or aromatic of one to about five rings, and optionally containing one or more heteroatoms and/or substituents. Q is a linker, typically a hydrocarbylene linker, including substituted hydrocarbylene, heteroatom-containing hydrocarbylene, and substituted heteroatom-containing hydrocarbylene linkers, wherein two or more substituents on adjacent atoms within Q may also be linked to form an additional cyclic structure, which may be similarly substituted to provide a fused polycyclic structure of two to about five cyclic groups. Q is often, although not necessarily, a two-atom linkage or a three-atom linkage.

Examples of N-heterocyclic carbene (NHC) ligands and acyclic diaminocarbene ligands suitable as L′ thus include, but are not limited to, the following where DIPP or DiPP is diisopropylphenyl and Mes is 2,4,6-trimethylphenyl:

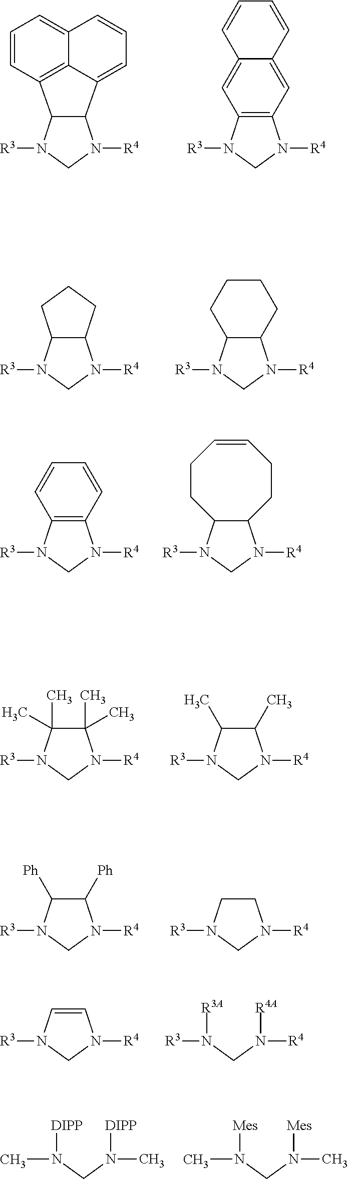

Additional examples of N-heterocyclic carbene (NHC) ligands and acyclic diaminocarbene ligands suitable as L′ thus include, but are not limited to the following:

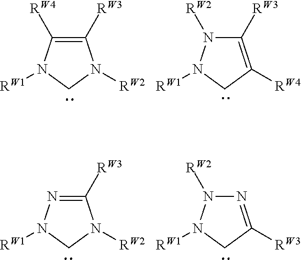

wherein R^{W1}, R^{W2}, R^{W3}, and R^{W4} are independently hydrogen, unsubstituted hydrocarbyl, substituted hydrocarbyl, or heteroatom containing hydrocarbyl, and where one or both of R^{W3} and R^{W4} may be in independently selected from halogen, nitro, amido, carboxyl, alkoxy, aryloxy, sulfonyl, carbonyl, thio, or nitroso groups.

Additional examples of N-heterocyclic carbene (NHC) ligands suitable as L¹ are further described in U.S. Pat. Nos. 7,378,528; 7,652,145; 7,294,717; 6,787,620; 6,635,768; and 6,552,139, the contents of each are incorporated herein by reference.

Additionally, thermally activated N-Heterocyclic Carbene Precursors as disclosed in U.S. Pat. No. 6,838,489, the contents of which are incorporated herein by reference, may also be used with the present invention.

When M is ruthenium, then the preferred complexes have the structure of formula (V)

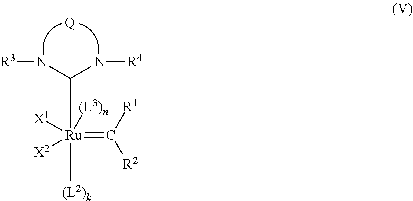

In a more preferred embodiment, Q is a two-atom linkage having the structure —CR¹¹R¹²—CR¹³R¹⁴— or —CR¹¹═CR¹³—, preferably —CR¹¹R¹²—CR¹³R¹⁴—, wherein R¹¹, R¹², R¹³, and R¹⁴ are independently selected from hydrogen, hydrocarbyl, substituted hydrocarbyl, heteroatom-containing hydrocarbyl, substituted heteroatom-containing hydrocarbyl, and functional groups. Examples of functional groups here include without limitation carboxyl, C₁-C₂₀ alkoxy, C₅-C₂₄ aryloxy, C₂-C₂₀ alkoxycarbonyl, C₅-C₂₄ alkoxycarbonyl, C₂-C₂₄ acyloxy, C₁-C₂₀ alkylthio, C₅-C₂₄ arylthio, C₁-C₂₀ alkylsulfonyl, and C₁-C₂₀ alkylsulfinyl, optionally substituted with one or more moieties selected from C₁-C₁₂ alkyl, C₁-C₁₂ alkoxy, C₅-C₁₄ aryl, hydroxyl, sulfhydryl, formyl, and halide. R¹¹, R¹², R¹³ and R¹⁴ are preferably independently selected from hydrogen, C₁-C₁₂ alkyl, substituted C₁-C₁₂ alkyl, C₁-C₁₂ heteroalkyl, substituted C₁-C₁₂ heteroalkyl, phenyl, and substituted phenyl. Alternatively, any two of R¹¹, R¹², R¹³, and R¹⁴ may be linked together to form a substituted or unsubstituted, saturated or unsaturated ring structure, e.g., a C₄-C₁₂ alicyclic group or a C₅ or C₆ aryl group, which may itself be substituted, e.g., with linked or fused alicyclic or aromatic groups, or with other substituents. In one further aspect, any one or more of R¹¹, R¹², R¹³, and R¹⁴ comprises one or more of the linkers. Additionally, R³ and R⁴ may be unsubstituted phenyl or phenyl substituted with one or more substituents selected from C₁-C₂₀ alkyl, substituted C₁-C₂₀ alkyl, C₁-C₂₀ heteroalkyl, substituted C₁-C₂₀ heteroalkyl, C₅-C₂₄ aryl, substituted C₅-C₂₄ aryl, C₅-C₂₄ heteroaryl, C₆-C₂₄ aralkyl, C₆-C₂₄ alkaryl, or halide. Furthermore, X¹ and X² may be halogen.

When R³ and R⁴ are aromatic, they are typically although not necessarily composed of one or two aromatic rings, which may or may not be substituted, e.g., R³ and R⁴ may be phenyl, substituted phenyl, biphenyl, substituted biphenyl, or the like. In one preferred embodiment, R³ and R⁴ are the same and are each unsubstituted phenyl or phenyl substituted with up to three substituents selected from C₁-C₂₀ alkyl, substituted C₁-C₂₀ alkyl, C₁-C₂₀ heteroalkyl, substituted C₁-C₂₀ heteroalkyl, C₅-C₂₄ aryl, substituted C₅-C₂₄ aryl, C₅-C₂₄ heteroaryl, C₆-C₂₄ aralkyl, C₆-C₂₄ alkaryl, or halide. Preferably, any substituents present are hydrogen, C₁-C₁₂ alkyl, C₁-C₁₂ alkoxy, C₅-C₁₄ aryl, substituted C₅-C₁₄ aryl, or halide. As an example, R³ and R⁴ are mesityl (i.e., Mes as defined herein).

In a third group of catalysts having the structure of formula (I), M, m, n, X¹, X², R¹, and R² are as defined for the first group of catalysts, L′ is a strongly coordinating neutral electron donor ligand such as any of those described for the first and second group of catalysts, and L² and L³ are weakly coordinating neutral electron donor ligands in the form of optionally substituted heterocyclic groups. Again, n is zero or 1, such that L³ may or may not be present. Generally, in the third group of catalysts, L² and L³ are optionally substituted five- or six-membered monocyclic groups containing 1 to 4, preferably 1 to 3, most preferably 1 to 2 heteroatoms, or are optionally substituted bicyclic or polycyclic structures composed of 2 to 5 such five- or six-membered monocyclic groups. If the heterocyclic group is substituted, it should not be substituted on a coordinating heteroatom, and any one cyclic moiety within a heterocyclic group will generally not be substituted with more than 3 substituents.

For the third group of catalysts, examples of L² and L³ include, without limitation, heterocycles containing nitrogen, sulfur, oxygen, or a mixture thereof.

Examples of nitrogen-containing heterocycles appropriate for L² and L³ include pyridine, bipyridine, pyridazine, pyrimidine, bipyridamine, pyrazine, 1,3,5-triazine, 1,2,4-triazine, 1,2,3-triazine, pyrrole, 2H-pyrrole, 3H-pyrrole, pyrazole, 2H-imidazole, 1,2,3-triazole, 1,2,4-triazole, indole, 3H-indole, 1H-isoindole, cyclopenta(b)pyridine, indazole, quinoline, bisquinoline, isoquinoline, bisisoquinoline, cinnoline, quinazoline, naphthyridine, piperidine, piperazine, pyrrolidine, pyrazolidine, quinuclidine, imidazolidine, picolylimine, purine, benzimidazole, bisimidazole, phenazine, acridine, and carbazole. Additionally, the nitrogen-containing heterocycles may be optionally substituted on a non-coordinating heteroatom with a non-hydrogen substituent.

Examples of sulfur-containing heterocycles appropriate for L² and L³ include thiophene, 1,2-dithiole, 1,3-dithiole, thiepin, benzo(b)thiophene, benzo(c)thiophene, thionaphthene, dibenzothiophene, 2H-thiopyran, 4H-thiopyran, and thioanthrene.

Examples of oxygen-containing heterocycles appropriate for L² and L³ include 2H-pyran, 4H-pyran, 2-pyrone, 4-pyrone, 1,2-dioxin, 1,3-dioxin, oxepin, furan, 2H-1-benzopyran, coumarin, coumarone, chromene, chroman-4-one, isochromen-1-one, isochromen-3-one, xanthene, tetrahydrofuran, 1,4-dioxan, and dibenzofuran.

Examples of mixed heterocycles appropriate for L² and L³ include isoxazole, oxazole, thiazole, isothiazole, 1,2,3-oxadiazole, 1,2,4-oxadiazole, 1,3,4-oxadiazole, 1,2,3,4-oxatriazole, 1,2,3,5-oxatriazole, 3H-1,2,3-dioxazole, 3H-1,2-oxathiole, 1,3-oxathiole, 4H-1,2-oxazine, 2H-1,3-oxazine, 1,4-oxazine, 1,2,5-oxathiazine, o-isooxazine, phenoxazine, phenothiazine, pyrano[3,4-b]pyrrole, indoxazine, benzoxazole, anthranil, and morpholine.

Preferred L² and L³ ligands are aromatic nitrogen-containing and oxygen-containing heterocycles, and particularly preferred L² and L³ ligands are monocyclic N-heteroaryl ligands that are optionally substituted with 1 to 3, preferably 1 or 2, substituents. Specific examples of particularly preferred L² and L³ ligands are pyridine and substituted pyridines, such as 3-bromopyridine, 4-bromopyridine, 3,5-dibromopyridine, 2,4,6-tribromopyridine, 2,6-dibromopyridine, 3-chloropyridine, 4-chloropyridine, 3,5-dichloropyridine, 2,4,6-trichloropyridine, 2,6-dichloropyridine, 4-iodopyridine, 3,5-diiodopyridine, 3,5-dibromo-4-methylpyridine, 3,5-dichloro-4-methylpyridine, 3,5-dimethyl-4-bromopyridine, 3,5-dimethylpyridine, 4-methylpyridine, 3,5-diisopropylpyridine, 2,4,6-trimethylpyridine, 2,4,6-triisopropylpyridine, 4-(tert-butyl)pyridine, 4-phenylpyridine, 3,5-diphenylpyridine, 3,5-dichloro-4-phenylpyridine, and the like.

In general, any substituents present on L² and/or L³ are selected from halo, C₁-C₂₀ alkyl, substituted C₁-C₂₀ alkyl, C₁-C₂₀ heteroalkyl, substituted C₁-C₂₀ heteroalkyl, C₅-C₂₄ aryl, substituted C₅-C₂₄ aryl, C₅-C₂₄ heteroaryl, substituted C₅-C₂₄ heteroaryl, C₆-C₂₄ alkaryl, substituted C₆-C₂₄ alkaryl, C₆-C₂₄ heteroalkaryl, substituted C₆-C₂₄ heteroalkaryl, C₆-C₂₄ aralkyl, substituted C₆-C₂₄ aralkyl, C₆-C₂₄ heteroaralkyl, substituted C₆-C₂₄ heteroaralkyl, and functional groups, with suitable functional groups including, without limitation, C₁-C₂₀ alkoxy, C₅-C₂₄ aryloxy, C₂-C₂₀ alkylcarbonyl, C₆-C₂₄ arylcarbonyl, C₂-C₂₀ alkylcarbonyloxy, C₆-C₂₄ arylcarbonyloxy, C₂-C₂₀ alkoxycarbonyl, C₆-C₂₄ aryloxycarbonyl, halocarbonyl, C₂-C₂₀ alkylcarbonato, C₆-C₂₄ arylcarbonato, carboxy, carboxylato, carbamoyl, mono-(C₁-C₂₀ alkyl)-substituted carbamoyl, di-(C₁-C₂₀ alkyl)-substituted carbamoyl, di-N—(C₁-C₂₀ alkyl), N—(C₅-C₂₄ aryl)-substituted carbamoyl, mono-(C₅-C₂₄ aryl)-substituted carbamoyl, di-(C₆-C₂₄ aryl)-substituted carbamoyl, thiocarbamoyl, mono-(C₁-C₂₀ alkyl)-substituted thiocarbamoyl, di-(C₁-C₂₀ alkyl)-substituted thiocarbamoyl, di-N—(C₁-C₂₀ alkyl)-N—(C₆-C₂₄ aryl)-substituted thiocarbamoyl, mono-(C₆-C₂₄ aryl)-substituted thiocarbamoyl, di-(C₆-C₂₄ aryl)-substituted thiocarbamoyl, carbamido, formyl, thioformyl, amino, mono-(C₁-C₂₀ alkyl)-substituted amino, di-(C₁-C₂₀ alkyl)-substituted amino, mono-(C₅-C₂₄ aryl)-substituted amino, di-(C₅-C₂₄ aryl)-substituted amino, di-N—(C₁-C₂₀ alkyl), N—(C₅-C₂₄ aryl)-substituted amino, C₂-C₂₀ alkylamido, C₆-C₂₄ arylamido, imino, C₁-C₂₀ alkylimino, C₅-C₂₄ arylimino, nitro, and nitroso. In addition, two adjacent substituents may be taken together to form a ring, generally a five- or six-membered alicyclic or aryl ring, optionally containing 1 to 3 heteroatoms and 1 to 3 substituents as above.

Preferred substituents on L² and L³ include, without limitation, halo, C₁-C₁₂ alkyl, substituted C₁-C₁₂ alkyl, C₁-C₁₂ heteroalkyl, substituted C₁-C₁₂ heteroalkyl, C₅-C₁₄ aryl, substituted C₅-C₁₄ aryl, C₅-C₁₄ heteroaryl, substituted C₅-C₁₄ heteroaryl, C₆-C₁₆ alkaryl, substituted C₆-C₁₆ alkaryl, C₆-C₁₆ heteroalkaryl, substituted C₆-C₁₆ heteroalkaryl, C₆-C₁₆ aralkyl, substituted C₆-C₁₆ aralkyl, C₆-C₁₆ heteroaralkyl, substituted C₆-C₁₆ heteroaralkyl, C₁-C₁₂ alkoxy, C₅-C₁₄ aryloxy, C₂-C₁₂ alkylcarbonyl, C₆-C₁₄ arylcarbonyl, C₂-C₁₂ alkylcarbonyloxy, C₆-C₁₄ arylcarbonyloxy, C₂-C₁₂ alkoxycarbonyl, C₆-C₁₄ aryloxycarbonyl, halocarbonyl, formyl, amino, mono-(C₁-C₁₂ alkyl)-substituted amino, di-(C₁-C₁₂ alkyl)-substituted amino, mono-(C₅-C₁₄ aryl)-substituted amino, di-(C₅-C₁₄ aryl)-substituted amino, and nitro.

Of the foregoing, the most preferred substituents are halo, C₁-C₆ alkyl, C₁-C₆ haloalkyl, C₁-C₆ alkoxy, phenyl, substituted phenyl, formyl, N,N-di(C₁-C₆ alkyl)amino, nitro, and nitrogen heterocycles as described above (including, for example, pyrrolidine, piperidine, piperazine, pyrazine, pyrimidine, pyridine, pyridazine, etc.).

In certain embodiments, L² and L³ may also be taken together to form a bidentate or multidentate ligand containing two or more, generally two, coordinating heteroatoms such as N, O, S, or P, with preferred such ligands being diimine ligands of the Brookhart type. One representative bidentate ligand has the structure of formula (VI)

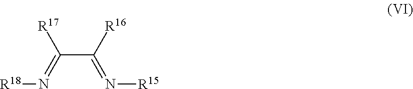

wherein R¹⁵, R¹⁶, R¹⁷, and R¹⁸ hydrocarbyl (e.g., C₁-C₂₀ alkyl, C₂-C₂₀ alkenyl, C₂-C₂₀ alkynyl, C₅-C₂₄ aryl, C₆-C₂₄ alkaryl, or C₆-C₂₄ aralkyl), substituted hydrocarbyl (e.g., substituted C₁-C₂₀ alkyl, C₂-C₂₀ alkenyl, C₂-C₂₀ alkynyl, C₅-C₂₄ aryl, C₆-C₂₄ alkaryl, or C₆-C₂₄ aralkyl), heteroatom-containing hydrocarbyl (e.g., C₁-C₂₀ heteroalkyl, C₅-C₂₄ heteroaryl, heteroatom-containing C₆-C₂₄ aralkyl, or heteroatom-containing C₆-C₂₄ alkaryl), or substituted heteroatom-containing hydrocarbyl (e.g., substituted C₁-C₂₀ heteroalkyl, C₅-C₂₄ heteroaryl, heteroatom-containing C₆-C₂₄ aralkyl, or heteroatom-containing C₆-C₂₄ alkaryl), or (1) R¹⁵ and R¹⁶, (2) R¹⁷ and R¹⁸, (3) R¹⁶ and R¹⁷, or (4) both R¹⁵ and R¹⁶, and R¹⁷ and R¹⁸, may be taken together to form a ring, i.e., an N-heterocycle. Preferred cyclic groups in such a case are five- and six-membered rings, typically aromatic rings.

In a fourth group of catalysts that have the structure of formula (I), two of the substituents are taken together to form a bidentate ligand or a tridentate ligand. Examples of bidentate ligands include, but are not limited to, bisphosphines, dialkoxides, alkyldiketonates, and aryldiketonates. Specific examples include —P(Ph)₂CH₂CH₂P(Ph)₂-, —As(Ph)₂CH₂CH₂As(Ph₂)-, —P(Ph)₂CH₂CH₂C(CF₃)₂O—, binaphtholate dianions, pinacolate dianions, —P(CH₃)₂(CH₂)₂P(CH₃)₂—, and —OC(CH₃)₂(CH₃)₂CO—. Preferred bidentate ligands are —P(Ph)₂CH₂CH₂P(Ph)₂- and —P(CH₃)₂(CH₂)₂P(CH₃)₂—. Tridentate ligands include, but are not limited to, (CH₃)₂NCH₂CH₂P(Ph)CH₂CH₂N(CH₃)₂. Other preferred tridentate ligands are those in which any three of X¹, X², (e.g., X¹, L¹, and L²) are taken together to be cyclopentadienyl, indenyl, or fluorenyl, each optionally substituted with C₂-C₂₀ alkenyl, C₂-C₂₀ alkynyl, C₁-C₂₀ alkyl, C₅-C₂₀ aryl, C₁-C₂₀ alkoxy, C₂-C₂₀ alkenyloxy, C₂-C₂₀ alkynyloxy, C₅-C₂₀ aryloxy, C₂-C₂₀ alkoxycarbonyl, C₁-C₂₀ alkylthio, C₁-C₂₀ alkylsulfonyl, or C₁-C₂₀ alkylsulfinyl, each of which may be further substituted with C₁-C₆ alkyl, halide, C₁-C₆ alkoxy, or with a phenyl group optionally substituted with halide, C₁-C₆ alkyl, or C₁-C₆ alkoxy. More preferably, in compounds of this type, X, L¹, and L² are taken together to be cyclopentadienyl or indenyl, each optionally substituted with vinyl, C₁-C₁₀ alkyl, C₅-C₂₀ aryl, C₁-C₁₀ carboxylate, C₂-C₁₀ alkoxycarbonyl, C₁-C₁₀ alkoxy, or C₅-C₂₀ aryloxy, each optionally substituted with C₁-C₆ alkyl, halide, C₁-C₆ alkoxy, or with a phenyl group optionally substituted with halide, C₁-C₆ alkyl, or C₁-C₆ alkoxy. Most preferably, X, L¹, and L² may be taken together to be cyclopentadienyl, optionally substituted with vinyl, hydrogen, methyl, or phenyl. Tetradentate ligands include, but are not limited to O₂C(CH₂)₂P(Ph)(CH₂)₂P(Ph)(CH₂)₂CO₂, phthalocyanines, and porphyrins.

Complexes wherein Y is coordinated to the metal are examples of a fifth group of catalysts, and are commonly called “Grubbs-Hoveyda” catalysts. Grubbs-Hoveyda metathesis-active metal carbene complexes may be described by the formula (VII)

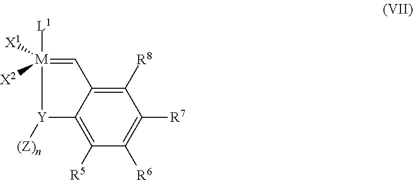

wherein,

M is a Group 8 transition metal, particularly Ru or Os, or, more particularly, Ru;

X¹, X², and L′ are as previously defined herein for the first and second groups of catalysts;

Y is a heteroatom selected from N, O, S, and P; preferably Y is O or N; R⁵, R⁶, R⁷, and R⁸ are each, independently, selected from the group consisting of hydrogen, halogen, alkyl, alkenyl, alkynyl, aryl, heteroalkyl, heteroatom containing alkenyl, heteroalkenyl, heteroaryl, alkoxy, alkenyloxy, aryloxy, alkoxycarbonyl, carbonyl, alkylamino, alkylthio, aminosulfonyl, monoalkylaminosulfonyl, dialkylaminosulfonyl, alkylsulfonyl, nitrile, nitro, alkylsulfinyl, trihaloalkyl, perfluoroalkyl, carboxylic acid, ketone, aldehyde, nitrate, cyano, isocyanate, hydroxyl, ester, ether, amine, imine, amide, halogen-substituted amide, trifluoroamide, sulfide, disulfide, sulfonate, carbamate, silane, siloxane, phosphine, phosphate, borate, or -A-Fn, wherein “A” and Fn have been defined above; and any combination of Y, Z, R⁵, R⁶, R⁷, and R⁸ can be linked to form one or more cyclic groups; n is 0, 1, or 2, such that n is 1 for the divalent heteroatoms O or S, and n is 2 for the trivalent heteroatoms N or P; and

Z is a group selected from hydrogen, alkyl, aryl, functionalized alkyl, functionalized aryl where the functional group(s) may independently be one or more or the following: halogen, carboxylic acid, ketone, aldehyde, nitrate, cyano, isocyanate, hydroxyl, ester, ether, amine, imine, amide, trifluoroamide, sulfide, disulfide, carbamate, silane, siloxane, phosphine, phosphate, or borate; methyl, isopropyl, sec-butyl, t-butyl, neopentyl, benzyl, phenyl, and trimethylsilyl; and wherein any combination or combinations of X¹, X², L¹, Y, Z, R⁵, R⁶, R⁷, and R⁸ may be linked to a support. Additionally, R⁵, R⁶, R⁷, R⁸, and Z may independently be thioisocyanate, cyanato, or thiocyanato.

In general, Grubbs-Hoveyda complexes useful in the invention contain a chelating alkylidene moiety of the formula (VIII)

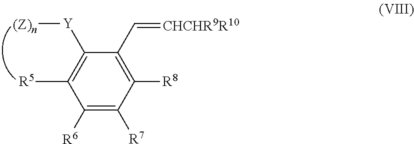

wherein Y, n, Z, R⁵, R⁶, R⁷, and R⁸ are as previously defined herein;

Y, Z, and R⁵ can optionally be linked to form a cyclic structure; and

R⁹ and R¹⁰ are each, independently, selected from hydrogen or a substituent group selected from alkyl, aryl, alkoxy, aryloxy, C₂-C₂₀ alkoxycarbonyl, or C₁-C₂₀ trialkylsilyl, wherein each of the substituent groups is substituted or unsubstituted; and wherein any combination or combinations of Z, Y, R⁵, R⁶, R⁷, R⁸, R⁹, and R¹⁰ may be linked to a support. The chelating alkylidene moiety may be derived from a ligand precursor having the formula (VIIIa)

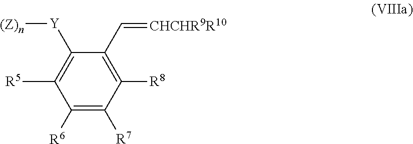

Examples of complexes comprising Grubbs-Hoveyda ligands suitable in the invention include:

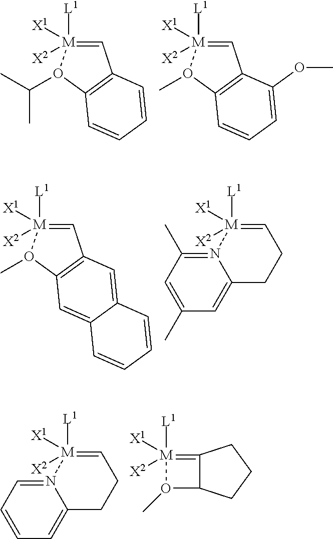

wherein, L¹, X¹, X², and M are as described for any of the other groups of catalysts. Suitable chelating carbenes and carbene precursors are further described by Pederson et al. (U.S. Pat. Nos. 7,026,495 and 6,620,955, the disclosures of both of which are incorporated herein by reference) and Hoveyda et al. (U.S. Pat. No. 6,921,735 and WO 02/14376, the disclosures of both of which are incorporated herein by reference).

Other useful complexes include structures wherein L² and R² according to formulae (I), (III), or (V) are linked, such as styrenic compounds that also include a functional group for attachment to a support. Examples in which the functional group is a trialkoxysilyl functionalized moiety include, but are not limited to, the following:

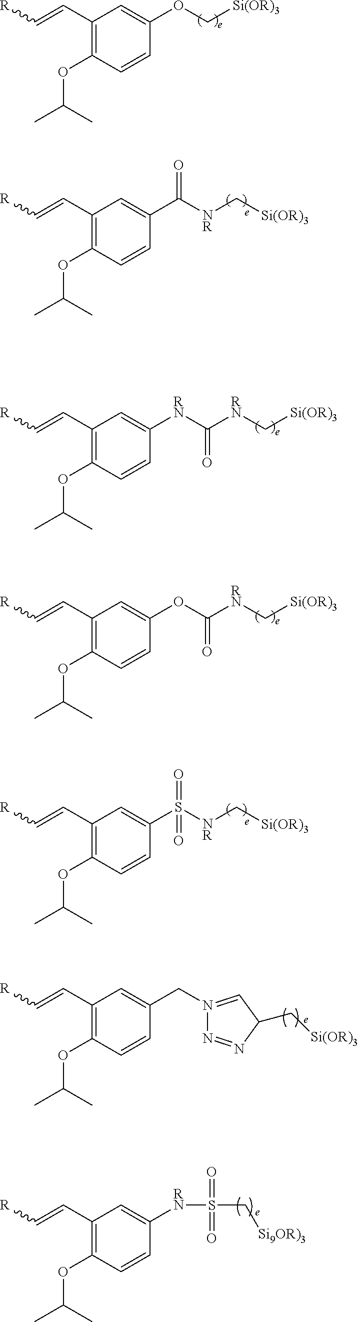

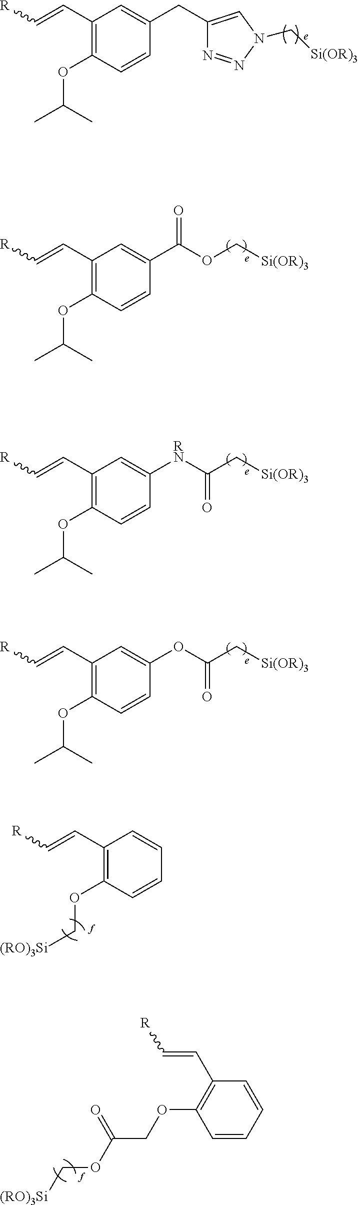

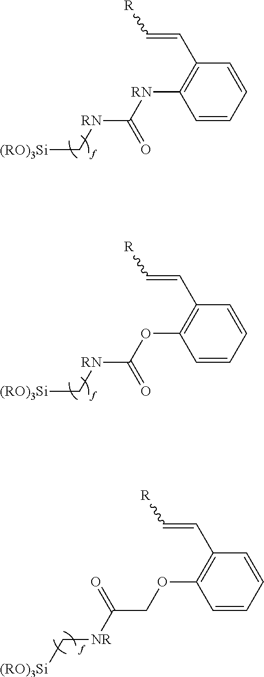

Further examples of complexes having linked ligands include those having linkages between a neutral NHC ligand and an anionic ligand, a neutral NHC ligand and an alkylidine ligand, a neutral NHC ligand and an L² ligand, a neutral NHC ligand and an L³ ligand, an anionic ligand and an alkylidine ligand, and any combination thereof. While the possible structures are too numerous to list herein, some suitable structures based on formula (III) include:

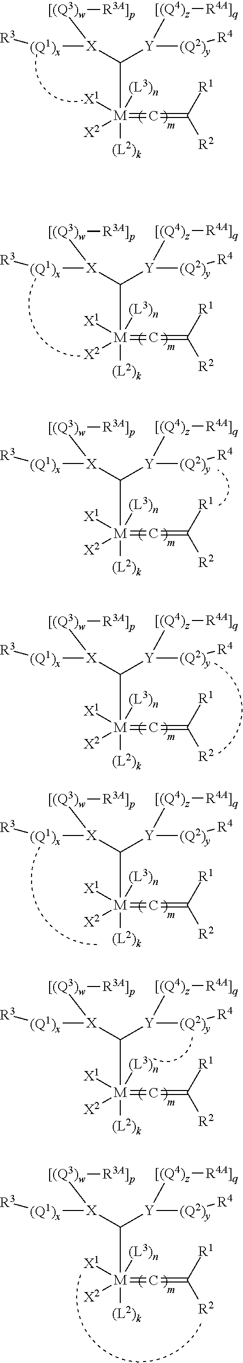

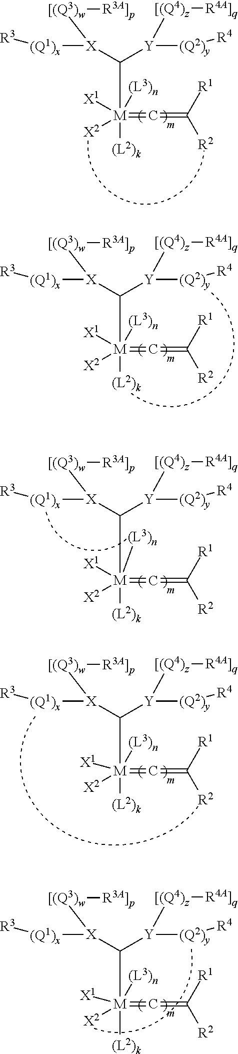

In addition to the catalysts that have the structure of formula (I), as described above, other transition metal carbene complexes include, but are not limited to: neutral ruthenium or osmium metal carbene complexes containing metal centers that are formally in the +2 oxidation state, have an electron count of 16, are penta-coordinated, and are of the general formula (IX); neutral ruthenium or osmium metal carbene complexes containing metal centers that are formally in the +2 oxidation state, have an electron count of 18, are hexa-coordinated, and are of the general formula (X); cationic ruthenium or osmium metal carbene complexes containing metal centers that are formally in the +2 oxidation state, have an electron count of 14, are tetra-coordinated, and are of the general formula (XI); and cationic ruthenium or osmium metal carbene complexes containing metal centers that are formally in the +2 oxidation state, have an electron count of 14 or 16, are tetra-coordinated or penta-coordinated, respectively, and are of the general formula (XII)

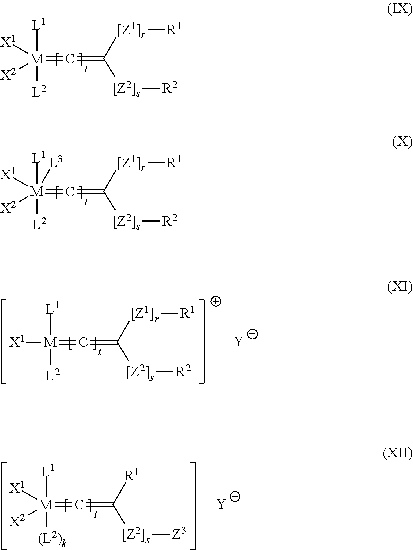

wherein:

M, X¹, X², L¹, L², L³, R¹, and R² are as defined for any of the previously defined four groups of catalysts;

r and s are independently zero or 1;

t is an integer in the range of zero to 5;

k is an integer in the range of zero to 1;

Y is any non-coordinating anion (e.g., a halide ion, BF₄⁻, etc.);

Z¹ and Z² are independently selected from —O—, —S—, —NR²—, —PR²—, —P(═O)R²—, —P(OR²)—, —P(═O)(OR²)—, —C(═O)—, —C(═O)O—, —OC(═O)—, —OC(═O)O—, —S(═O)—, —S(═O)₂—, and an optionally substituted and/or optionally heteroatom-containing C₁-C₂₀ hydrocarbylene linkage; Z³ is any cationic moiety such as —P(R²)₃⁺ or —N(R²)₃⁺; and any two or more of X¹, X², L¹, L², L³, Z¹, Z², Z³, R¹, and R² may be taken together to form a cyclic group, e.g., a multidentate ligand, and wherein any one or more of X¹, X², L¹, L², L³, Z¹, Z², Z³, R¹, and R² may be attached to a support.

Additionally, another group of olefin metathesis catalysts that may be used in the invention disclosed herein, is a Group 8 transition metal complex having the structure of formula (XIII):

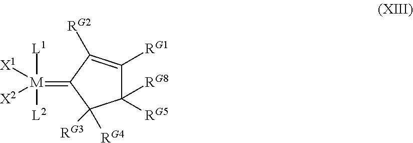

wherein M is a Group 8 transition metal, particularly ruthenium or osmium, or more particularly, ruthenium;

X¹, X², L¹, and L² are as defined for the first and second groups of catalysts defined above; and

R^{G1}, R^{G2}, R^{G3}, R^{G4}, R^{G5}, and R^{G6} are each independently selected from the group consisting of hydrogen, halogen, alkyl, alkenyl, alkynyl, aryl, heteroalkyl, heteroatom containing alkenyl, heteroalkenyl, heteroaryl, alkoxy, alkenyloxy, aryloxy, alkoxycarbonyl, carbonyl, alkylamino, alkylthio, aminosulfonyl, monoalkylaminosulfonyl, dialkylaminosulfonyl, alkylsulfonyl, nitrile, nitro, alkylsulfinyl, trihaloalkyl, perfluoroalkyl, carboxylic acid, ketone, aldehyde, nitrate, cyano, isocyanate, thioisocyanate, cyanato, thiocyanato, hydroxyl, ester, ether, thioether, amine, alkylamine, imine, amide, halogen-substituted amide, trifluoroamide, sulfide, disulfide, sulfonate, carbamate, silane, siloxane, phosphine, phosphate, borate, or -A-Fn, wherein “A” is a divalent hydrocarbon moiety selected from alkylene and arylalkylene, wherein the alkyl portion of the alkylene and arylalkylene groups can be linear or branched, saturated or unsaturated, cyclic or acyclic, and substituted or unsubstituted, wherein the aryl portion of the arylalkylene can be substituted or unsubstituted, and wherein hetero atoms and/or functional groups may be present in either the aryl or the alkyl portions of the alkylene and arylalkylene groups, and Fn is a functional group, or any one or more of the R^{G1}, R^{G2}, R^{G3}, R^{G4}, R^{G5}, and R^{G6} may be linked together to form a cyclic group, or any one or more of the R^{G1}, R^{G2}, R^{G3}, R^{G4}, R^{G5}, and R^{G6} may be attached to a support.

Additionally, one preferred embodiment of the Group 8 transition metal complex of formula XIII is a Group 8 transition metal complex of formula (XIV):

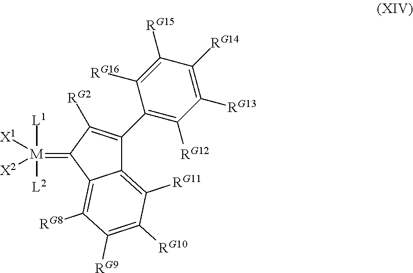

wherein M, X¹, X², L¹, and L² are as defined above for Group 8 transition metal complex of formula XIII; and R^{G7}, R^{G8}, R^{G9}, R^{G10}, R^{G11}, R^{G12}, R^{G13}, R^{G14}, R^{G15}, and R^{G16} are as defined above for R^{G1}, R^{G2}, R^{G3}, R^{G4}, R^{G5}, and R^{G6} for Group 8 transition metal complex of formula XIII or any one or more of the R^{G7}, R^{G8}, R^{G9}, R^{G10}, R^{G11}, R^{G12}, R^{G13}, R^{G14}, R^{G15}, and R^{G16} may be linked together to form a cyclic group, or any one or more of the R^{G1}, R^{G8}, R^{G9}, R^{G10}, R^{G11}, R^{G12}, R^{G13}, R^{G14}, R^{G15}, and R^{G16} may be attached to a support.

Additionally, another preferred embodiment of the Group 8 transition metal complex of formula XIII is a Group 8 transition metal complex of formula (XV):

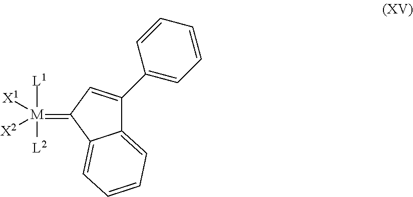

wherein M, X¹, X², L¹, and L² are as defined above for Group 8 transition metal complex of formula XIII.

Additionally, another group of olefin metathesis catalysts that may be used in the invention disclosed herein is a Group 8 transition metal complex comprising a Schiff base ligand having the structure of formula (XVI):

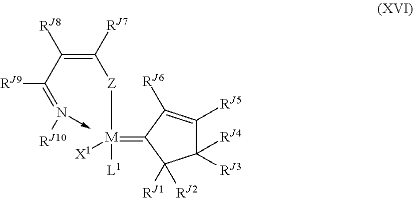

wherein M is a Group 8 transition metal, particularly ruthenium or osmium, or more particularly, ruthenium;

X¹ and L¹ are as defined for the first and second groups of catalysts defined above;

Z is selected from the group consisting of oxygen, sulfur, selenium, NR^{J11} PR^{J11}, AsR^{J11}, and SbR^{J11}; and R^{J1}, R^{J2}, R^{J3}, R^{J4}, R^{J5}, R^{J6}, R^{J7}, R^{J8}, R^{J9}, R^{J10}, and R^{J11} are each independently selected from the group consisting of hydrogen, halogen, alkyl, alkenyl, alkynyl, aryl, heteroalkyl, heteroatom containing alkenyl, heteroalkenyl, heteroaryl, alkoxy, alkenyloxy, aryloxy, alkoxycarbonyl, carbonyl, alkylamino, alkylthio, aminosulfonyl, monoalkylaminosulfonyl, dialkylaminosulfonyl, alkylsulfonyl, nitrile, nitro, alkylsulfinyl, trihaloalkyl, perfluoroalkyl, carboxylic acid, ketone, aldehyde, nitrate, cyano, isocyanate, thioisocyanate, cyanato, thiocyanato, hydroxyl, ester, ether, thioether, amine, alkylamine, imine, amide, halogen-substituted amide, trifluoroamide, sulfide, disulfide, sulfonate, carbamate, silane, siloxane, phosphine, phosphate, borate, or -A-Fn, wherein “A” is a divalent hydrocarbon moiety selected from alkylene and arylalkylene, wherein the alkyl portion of the alkylene and arylalkylene groups can be linear or branched, saturated or unsaturated, cyclic or acyclic, and substituted or unsubstituted, wherein the aryl portion of the arylalkylene can be substituted or unsubstituted, and wherein hetero atoms and/or functional groups may be present in either the aryl or the alkyl portions of the alkylene and arylalkylene groups, and Fn is a functional group, or any one or more of the R^{J1}, R^{J2}, R^{J3}, R^{J4}, R^{J5}, R^{J6}, R^{J7}, R^{J8}, R^{J9}, R^{J10}, and R^{J11} may be linked together to form a cyclic group, or any one or more of the R^{J1}, R^{J2}, R^{J3}, R^{J4}, R^{J5}, R^{J6}, R^{J7}, R^{J8}, R^{J9}, R^{J10}, and R^{J11} may be attached to a support.

Additionally, one preferred embodiment of the Group 8 transition metal complex of formula (XVI) is a Group 8 transition metal complex comprising a Schiff base ligand having the structure of formula (XVII):

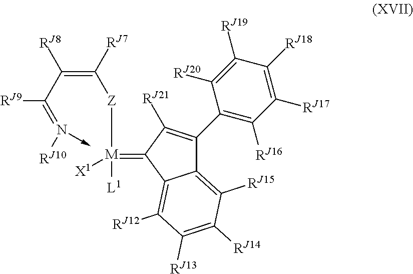

wherein M, X¹, L¹, Z, R^{J7}, R^{J8}, R^{J9}, R^{J10}, and R^{J11} are as defined above for Group 8 transition metal complex of formula XVI; and R^{J12}, R^{J13}, R^{J14}, R^{J15}, R^{J16}, R^{J17}, R^{J18}, R^{J19}, R^{J20}, and R^{J21} are as defined above for R^{J1}, R^{J2}, R^{J3}, R^{J4}, R^{J5}, and R^{J6} for Group 8 transition metal complex of formula XVI, or any one or more of the R^{J7}, R^{J8}, R^{J9}, R^{J10}, R^{J11}, R^{J12}, R^{J13}, R^{J14}, R^{J15}, R^{J16}, R^{J17}, R^{J18}, R^{J19}, R^{J20}, and R^{J21} may be linked together to form a cyclic group, or any one or more of the R^{J7}, R^{J8}, R^{J9}, R^{J10}, R^{J11}, R^{J12}, R^{J13}, R^{J14}, R^{J15}, R^{J16}, R^{J17}, R^{J18}, R^{J19}, R^{J20}, and R^{J21} may be attached to a support.

Additionally, another preferred embodiment of the Group 8 transition metal complex of formula (XVI) is a Group 8 transition metal complex comprising a Schiff base ligand having the structure of formula (XVIII):

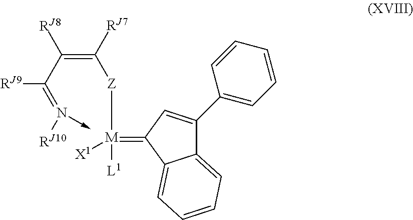

wherein M, X¹, L¹, Z, R^{J7}, R^{J8}, R^{J9}, R^{J10}, and R^{III} are as defined above for Group 8 transition metal complex of formula (XVI).

Additionally, another group of olefin metathesis catalysts that may be used in the invention disclosed herein is a Group 8 transition metal complex comprising a Schiff base ligand having the structure of formula (XIX):

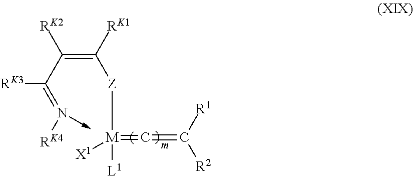

wherein M is a Group 8 transition metal, particularly ruthenium or osmium, or more particularly, ruthenium;

X¹, L¹, R¹, and R² are as defined for the first and second groups of catalysts defined above;

Z is selected from the group consisting of oxygen, sulfur, selenium, NR^{K5}, PR^{K5}, AsR^{K5}, and SbR^{K5};
m is 0, 1, or 2; and

R^{K1}, R^{K2}, R^{K3}, R^{K4}, and R^{K5} are each independently selected from the group consisting of hydrogen, halogen, alkyl, alkenyl, alkynyl, aryl, heteroalkyl, heteroatom containing alkenyl, heteroalkenyl, heteroaryl, alkoxy, alkenyloxy, aryloxy, alkoxycarbonyl, carbonyl, alkylamino, alkylthio, aminosulfonyl, monoalkylaminosulfonyl, dialkylaminosulfonyl, alkylsulfonyl, nitrile, nitro, alkylsulfinyl, trihaloalkyl, perfluoroalkyl, carboxylic acid, ketone, aldehyde, nitrate, cyano, isocyanate, thioisocyanate, cyanato, thiocyanato, hydroxyl, ester, ether, thioether, amine, alkylamine, imine, amide, halogen-substituted amide, trifluoroamide, sulfide, disulfide, sulfonate, carbamate, silane, siloxane, phosphine, phosphate, borate, or -A-Fn, wherein “A” is a divalent hydrocarbon moiety selected from alkylene and arylalkylene, wherein the alkyl portion of the alkylene and arylalkylene groups can be linear or branched, saturated or unsaturated, cyclic or acyclic, and substituted or unsubstituted, wherein the aryl portion of the arylalkylene can be substituted or unsubstituted, and wherein hetero atoms and/or functional groups may be present in either the aryl or the alkyl portions of the alkylene and arylalkylene groups, and Fn is a functional group, or any one or more of the R^{K1}, R^{K2}, R^{K3}, R^{K4} and R^{K5}, may be linked together to form a cyclic group, or any one or more of the R^{K1}, R^{K2}, R^{K3}, R^{K4} and R^{K5}, may be attached to a support.

In addition, catalysts of formulas (XVI) to (XIX) may be optionally contacted with an activating compound, where at least partial cleavage of a bond between the Group 8 transition metal and at least one Schiff base ligand occurs, wherein the activating compound is either a metal or silicon compound selected from the group consisting of copper (I) halides; zinc compounds of the formula Zn(R^{Y1})₂, wherein R^{Y1} is halogen, C₁-C₇ alkyl or aryl; tin compounds represented by the formula SnR^{Y2}R^{Y3}R^{Y4}R^{Y5} wherein each of R^{Y2}, R^{Y3}, R^{Y4}, and R^{Y5} is independently selected from the group consisting of halogen, C₁-C₂₀ alkyl, C₃-C₁₀ cycloalkyl, aryl, benzyl, and C₂-C₇ alkenyl; and silicon compounds represented by the formula SiR^{Y6}R^{Y7}R^{Y8}R^{Y9} wherein each of R^{Y6}, R^{Y7}, R^{Y8}, and R^{Y9} is independently selected from the group consisting of hydrogen, halogen, C₁-C₂₀ alkyl, halo, C₁-C₇ alkyl, aryl, heteroaryl, and vinyl. In addition, catalysts of formulas (XVI) to (XIX) may be optionally contacted with an activating compound where at least partial cleavage of a bond between the Group 8 transition metal and at least one Schiff base ligand occurs, wherein the activating compound is an inorganic acid such as hydrogen iodide, hydrogen bromide, hydrogen chloride, hydrogen fluoride, sulfuric acid, nitric acid, iodic acid, periodic acid, perchloric acid, HOClO, HOClO₂ and HOIO₃. In addition, catalysts of formulas (XVI) to (XIX) may be optionally contacted with an activating compound where at least partial cleavage of a bond between the Group 8 transition metal and at least one Schiff base ligand occurs, wherein the activating compound is an organic acid such as sulfonic acids including but not limited to methanesulfonic acid, aminobenzenesulfonic acid, benzenesulfonic acid, napthalenesulfonic acid, sulfanilic acid and trifluoromethanesulfonic acid; monocarboxylic acids including but not limited to acetoacetic acid, barbituric acid, bromoacetic acid, bromobenzoic acid, chloroacetic acid, chlorobenzoic acid, chlorophenoxyacetic acid, chloropropionic acid, cis-cinnamic acid, cyanoacetic acid, cyanobutyric acid, cyanophenoxyacetic acid, cyanopropionic acid, dichloroacetic acid, dichloroacetylacetic acid, dihydroxybenzoic acid, dihydroxymalic acid, dihydroxytartaric acid, dinicotinic acid, diphenylacetic acid, fluorobenzoic acid, formic acid, furancarboxylic acid, furoic acid, glycolic acid, hippuric acid, iodoacetic acid, iodobenzoic acid, lactic acid, lutidinic acid, mandelic acid, α-naphtoic acid, nitrobenzoic acid, nitrophenylacetic acid, o-phenylbenzoic acid, thioacetic acid, thiophene-carboxylic acid, trichloroacetic acid, and trihydroxybenzoic acid; and other acidic substances such as but not limited to picric acid and uric acid.

In addition, other examples of catalysts that may be used with the present invention are located in the following disclosures, each of which is incorporated herein by reference, U.S. Pat. Nos. 7,687,635; 7,671,224; 6,284,852; 6,486,279; and 5,977,393; International Publication Number WO 2010/037550; and U.S. patent application Ser. Nos. 12/303,615; 10/590,380; 11/465,651 (U.S. Pat. App. Pub. No. 2007/0043188); and 11/465,651 (U.S. Pat. App. Pub. No. 2008/0293905 Corrected Publication); and European Pat. Nos. EP 1757613B1 and EP 1577282B1.

Non-limiting examples of catalysts that may be used to prepare supported complexes and in the reactions disclosed herein include the following, some of which for convenience are identified throughout this disclosure by reference to their molecular weight:

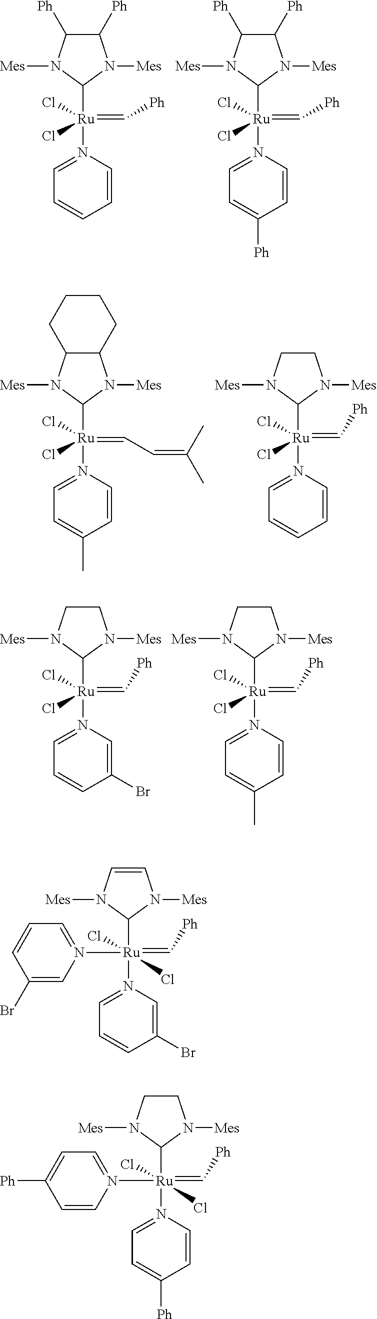

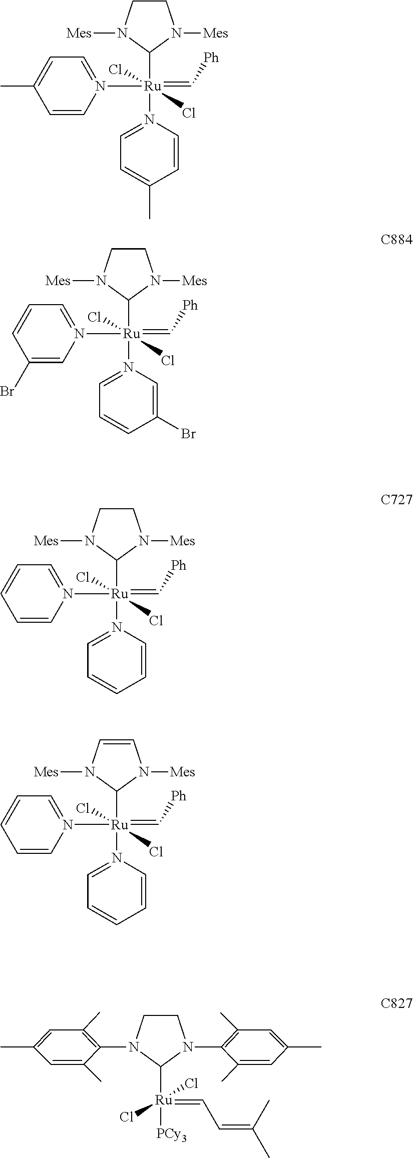

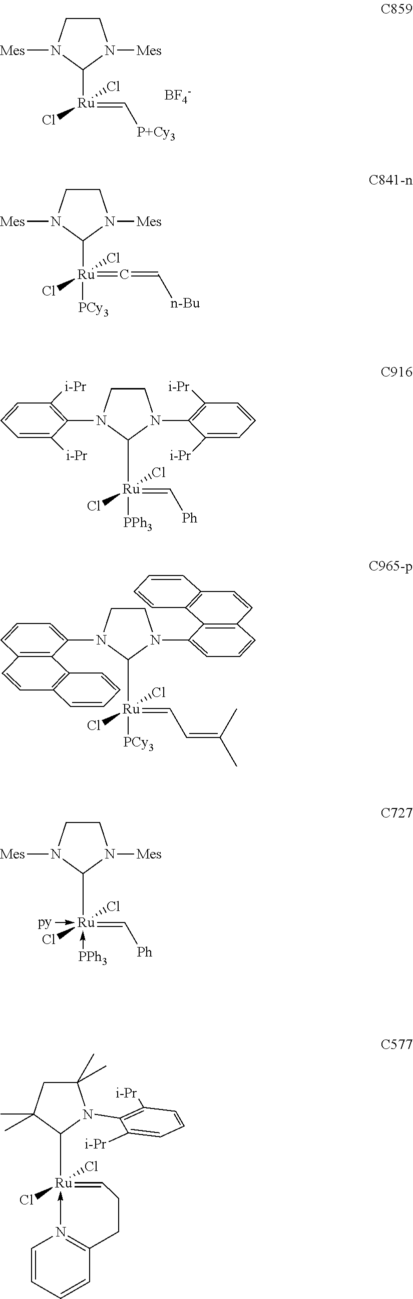

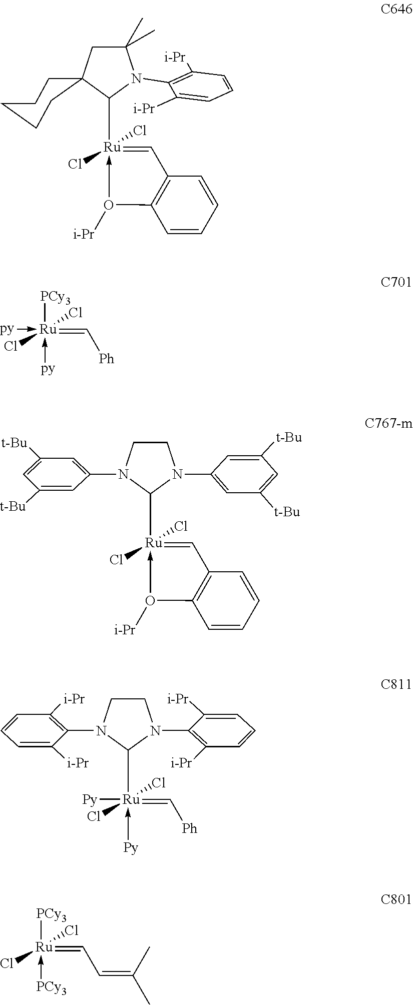

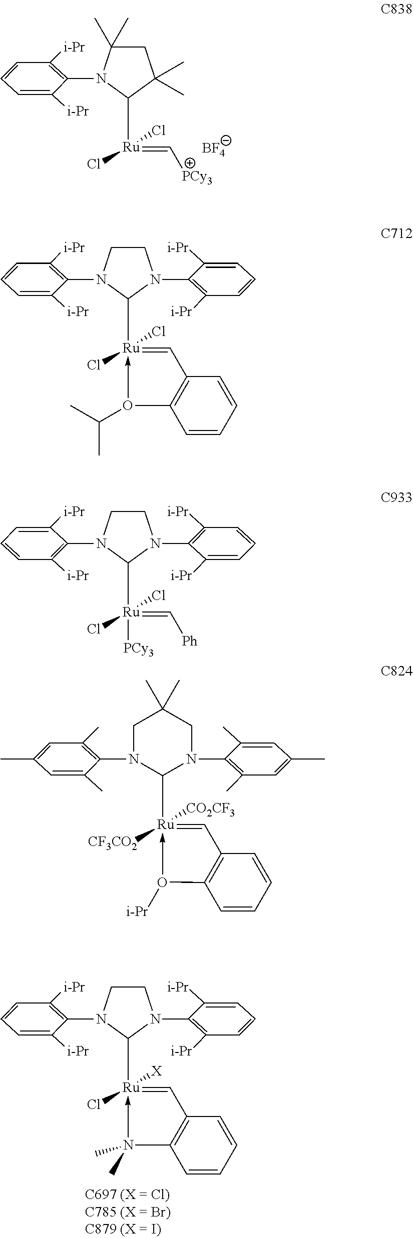

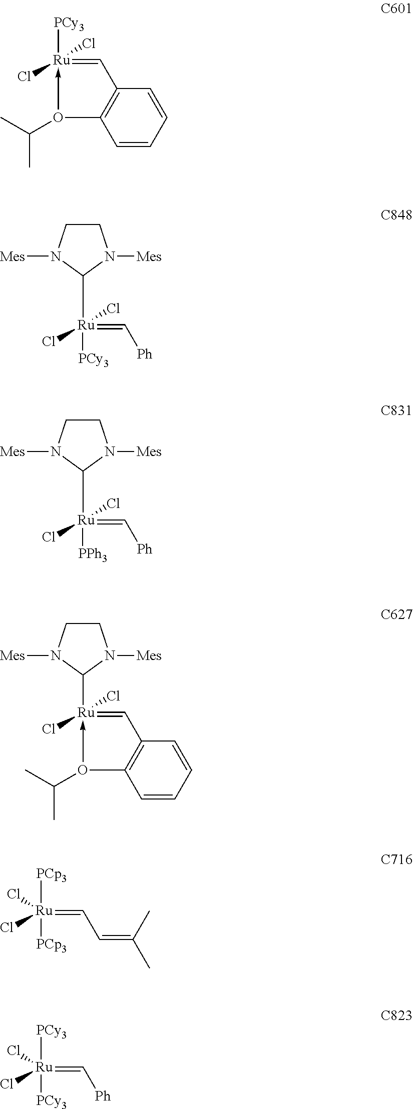

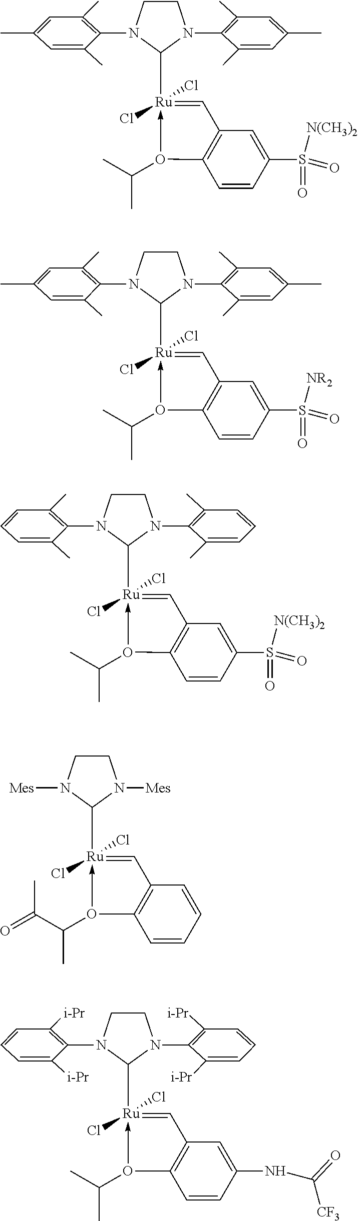

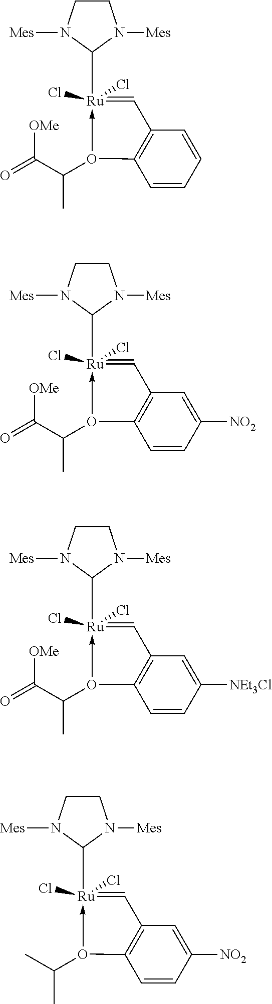

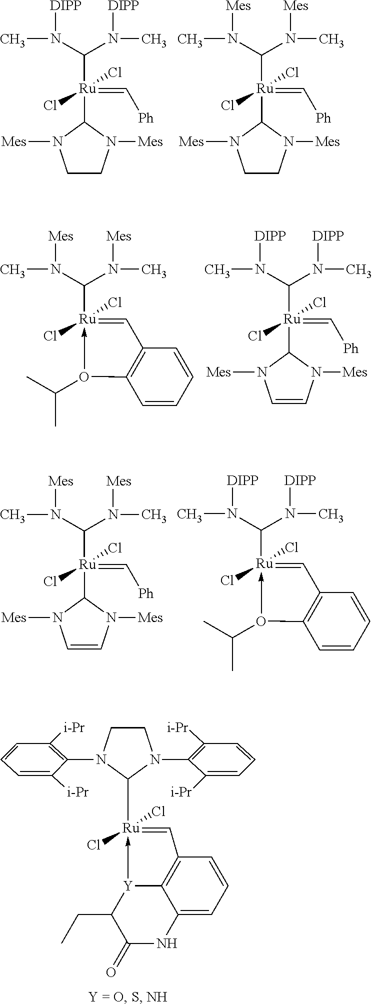

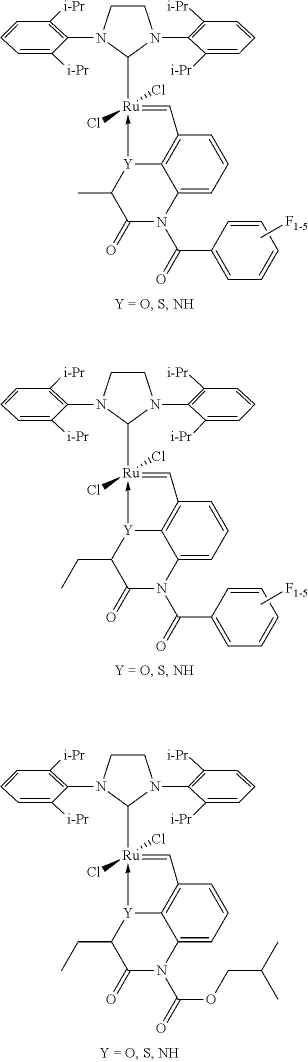

In the foregoing molecular structures and formulae, Ph represents phenyl, Cy represents cyclohexyl, Cp represents cyclopentyl, Me represents methyl, Bu represents n-butyl, t-Bu represents tert-butyl, i-Pr represents isopropyl, py represents pyridine (coordinated through the N atom), Mes represents mesityl (i.e., 2,4,6-trimethylphenyl), DiPP and DIPP represents 2,6-diisopropylphenyl, and MiPP represents 2-isopropylphenyl.

Further examples of catalysts useful to prepare supported complexes and in the reactions disclosed herein include the following: ruthenium (II) dichloro(3-methyl-2-butenylidene)bis(tricyclopentylphosphine) (C716); ruthenium (II) dichloro(3-methyl-2-butenylidene)bis(tricyclohexylphosphine) (C801); ruthenium (II) dichloro(phenylmethylene)bis(tricyclohexylphosphine) (C823); ruthenium (II) (1,3-bis-(2,4,6-trimethylphenyl)-2-imidazolidinylidene)dichloro(phenylmethylene) (triphenylphosphine) (C830); ruthenium (II) dichloro phenylvinylidene)bis(tricyclohexylphosphine) (C835); ruthenium (II) dichloro(tricyclohexylphosphine)(o-isopropoxyphenylmethylene) (C601); ruthenium (II) (1,3-bis-(2,4,6-trimethylphenyl)-2-imidazolidinylidene)dichloro(phenylmethylene)bis(3-bromopyridine) (C884); [1,3-bis-(2,4,6-trimethylphenyl)-2-imidazolidinylidene]dichloro(o-isopropoxyphenylmethylene)ruthenium(II) (C627); [1,3-bis-(2,4,6-trimethylphenyl)-2-imidazolidinylidene]dichloro(benzylidene) (triphenylphosphine) ruthenium(II) (C831); [1,3-bis-(2,4,6-trimethylphenyl)-2-imidazolidinylidene]dichloro(benzylidene)(methyldiphenylphosphine)ruthenium(II) (C769); [1,3-bis-(2,4,6-trimethylphenyl)-2-imidazolidinylidene]dichloro(benzylidene) (tricyclohexylphosphine)ruthenium(II) (C848); [1,3-bis-(2,4,6-trimethylphenyl)-2-imidazolidinylidene]dichloro(benzylidene) (diethylphenylphosphine) ruthenium(II) (C735); [1,3-bis-(2,4,6-trimethylphenyl)-2-imidazolidinylidene]dichloro(benzylidene)(tri-n-butylphosphine)ruthenium(II) (C771); [1,3-bis-(2,4,6-trimethylphenyl)-2-imidazolidinylidene]dichloro(3-methyl-2-butenylidene)(triphenylphosphine) ruthenium(II) (C809); [1,3-bis-(2,4,6-trimethylphenyl)-2-imidazolidinylidene]dichloro(3-methyl-2-butenylidene)(methyldiphenylphosphine)ruthenium(II) (C747); [1,3-bis-(2,4,6-trimethylphenyl)-2-imidazolidinylidene]dichloro(3-methyl-2-butenylidene) (tricyclohexylphosphine) ruthenium(II) (C827); [1,3-bis-(2,4,6-trimethylphenyl)-2-imidazolidinylidene]dichloro(3-methyl-2-butenylidene)(diethylphenylphosphine) ruthenium(II) (C713); [1,3-bis-(2,4,6-trimethylphenyl)-2-imidazolidinylidene]dichloro(3-methyl-2-butenylidene)(tri-n-butylphosphine)ruthenium(II) (C749); [1,3-bis-(2,4,6-trimethylphenyl)-2-imidazolidinylidene]dichloro(phenylindenylidene)(triphenylphosphine) ruthenium(II) (C931); [1,3-bis-(2,4,6-trimethylphenyl)-2-imidazolidinylidene]dichloro(phenylindenylidene)(methyldiphenylphosphine)ruthenium(II) (C869); [1,3-bis-(2,4,6-trimethylphenyl)-2-imidazolidinylidene]dichloro(phenylindenylidene)(tricyclohexylphosphine)ruthenium(II) (C949); [1,3-bis-(2,4,6-trimethylphenyl)-2-imidazolidinylidene]dichloro(phenylindenylidene) (diethylphenylphosphine)ruthenium(II) (C835); and [1,3-bis-(2,4,6-trimethylphenyl)-2-imidazolidinylidene]dichloro(phenylindenylidene)(tri-n-butylphosphine)ruthenium(II) (C871).

Still further catalysts useful in ROMP reactions, and/or in other metathesis reactions, such as ring-closing metathesis, cross metathesis, ring-opening cross metathesis, self-metathesis, ethenolysis, alkenolysis, acyclic diene metathesis polymerization, and combinations thereof, include the following structures:

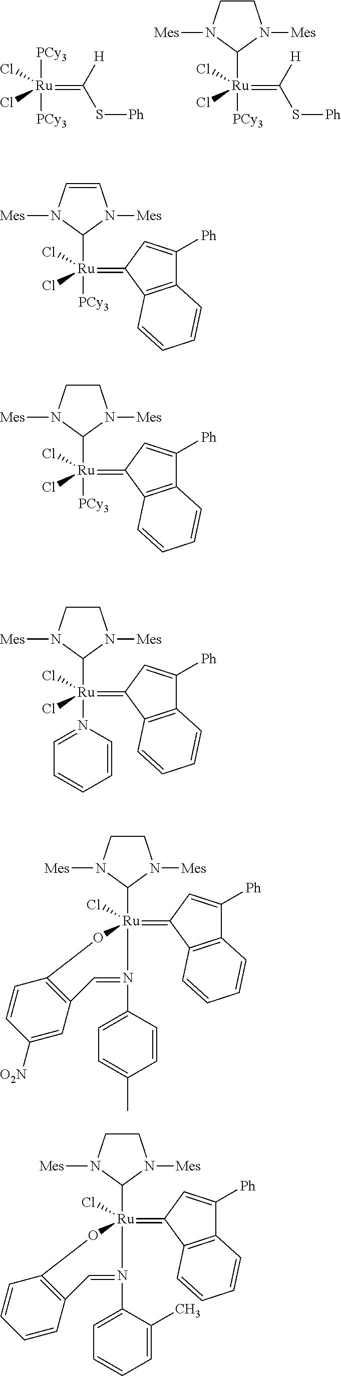

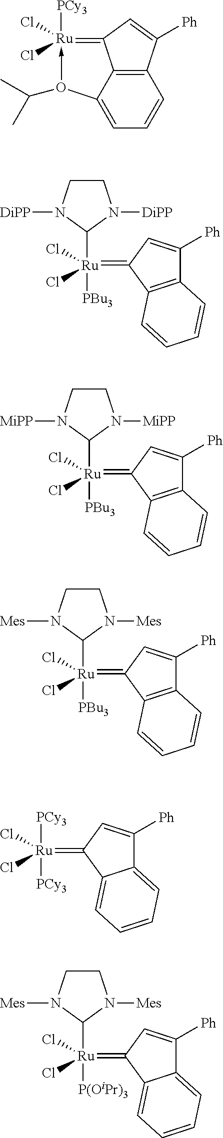

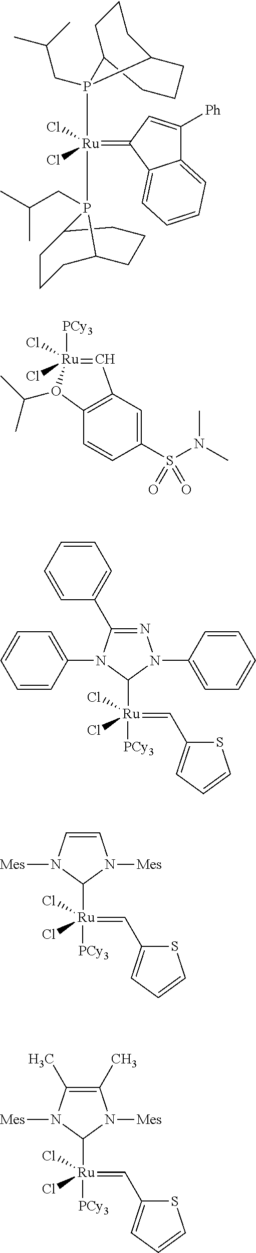

In general, the transition metal complexes used as catalysts herein can be prepared by several different methods, such as those described by Schwab et al. (1996) J. Am. Chem. Soc. 118:100-110, Scholl et al. (1999) Org. Lett. 6:953-956, Sanford et al. (2001) J. Am. Chem. Soc. 123:749-750, U.S. Pat. No. 5,312,940, and U.S. Pat. No. 5,342,909, the disclosures of each of which are incorporated herein by reference. Also see U.S. Pat. App. Pub. No. 2003/0055262 to Grubbs et al., WO 02/079208, and U.S. Pat. No. 6,613,910 to Grubbs et al., the disclosures of each of which are incorporated herein by reference. Preferred synthetic methods are described in WO 03/11455A1 to Grubbs et al., the disclosure of which is incorporated herein by reference.

Preferred olefin metathesis catalysts are Group 8 transition metal complexes having the structure of formula (I) commonly called “First Generation Grubbs” catalysts, formula (III) commonly called “Second Generation Grubbs” catalysts, or formula (VII) commonly called “Grubbs-Hoveyda” catalysts.

More preferred olefin metathesis catalysts have the structure of formula (I)

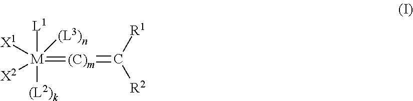

in which:

M is a Group 8 transition metal;

L¹, L², and L³ are neutral electron donor ligands;

n is 0 or 1;

m is 0, 1, or 2;

k is 0 or 1;

X¹ and X² are anionic ligands;

R¹ and R² are independently selected from hydrogen, hydrocarbyl, substituted hydrocarbyl, heteroatom-containing hydrocarbyl, substituted heteroatom-containing hydrocarbyl, and functional groups,

wherein any two or more of X¹, X², L¹, L², L³, R¹, and R² can be taken together to form one or more cyclic groups, and further wherein any one or more of X¹, X², L¹, L², L³, R^{X}, and R² may be attached to a support;

and formula (VII)

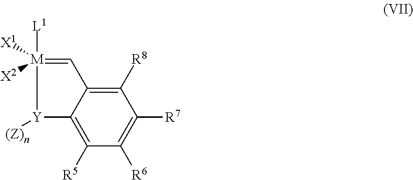

wherein,

M is a Group 8 transition metal;

L¹ is a neutral electron donor ligand;

X¹ and X² are anionic ligands;

Y is a heteroatom selected from O or N;

R⁵, R⁶, R⁷, and R⁸ are independently selected from hydrogen, hydrocarbyl, substituted hydrocarbyl, heteroatom-containing hydrocarbyl, substituted heteroatom-containing hydrocarbyl, and functional groups;

n is 0, 1, or 2; and

Z is selected from hydrogen, hydrocarbyl, substituted hydrocarbyl, heteroatom-containing hydrocarbyl, substituted heteroatom-containing hydrocarbyl, and functional groups, wherein any combination of Y, Z, R⁵, R⁶, R⁷, and R⁸ can be linked to form one or more cyclic groups, and further wherein any combination of X¹, X², L¹, Y, Z, R⁵, R⁶, R⁷, and R⁸ may be attached to a support.

Most preferred olefin metathesis catalysts have the structure of formula (I)

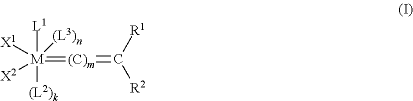

in which:

M is ruthenium;

n is 0;

m is 0;

k is 1;

L¹ and L² are trisubstituted phosphines independently selected from the group consisting of tri-n-butylphosphine (Pn-Bu₃), tricyclopentylphosphine (PCp₃), tricyclohexylphosphine (PCy₃), triisopropylphosphine (P-i-Pr₃), triphenylphosphine (PPh₃), methyldiphenylphosphine (PMePh₂), dimethylphenylphosphine (PMe₂Ph), and diethylphenylphosphine (PEt₂Ph); or L¹ is an N-heterocyclic carbene selected from the group consisting of 1,3-bis(2,4,6-trimethylphenyl)-2-imidazolidinylidene, 1,3-bis(2,4,6-trimethylphenyl) imidazol-2-ylidene, 1,3-bis(2,6-di-isopropylphenyl)-2-imidazolidinylidene, and 1,3-bis(2,6-di-isopropylphenyl)imidazol-2-ylidene, and L² is a trisubstituted phosphine selected from the group consisting of tri-n-butylphosphine (Pn-Bu₃), tricyclopentylphosphine (PCp₃), tricyclohexylphosphine (PCy₃), triisopropylphosphine (P-i-Pr₃), triphenylphosphine (PPh₃), methyldiphenylphosphine (PMePh₂), dimethylphenylphosphine (PMe₂Ph), and diethylphenylphosphine (PEt₂Ph);

X¹ and X² are chloride;

R¹ is hydrogen and R² is phenyl or —CH═C(CH₃)₂ or thienyl; or R¹ and R² are taken together to form 3-phenyl-1H-indene;

and formula (VII)

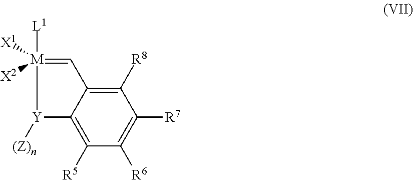

wherein,

M is ruthenium;

L¹ is a trisubstituted phosphine selected from the group consisting of tri-n-butylphosphine (Pn-Bu₃), tricyclopentylphosphine (PCp₃), tricyclohexylphosphine (PCy₃), triisopropylphosphine (P-i-Pr₃), triphenylphosphine (PPh₃), methyldiphenylphosphine (PmePh₂), dimethylphenylphosphine (Pme₂Ph), and diethylphenylphosphine (PEt₂Ph); or L′ is an N-heterocyclic carbene selected from the group consisting of 1,3-bis(2,4,6-trimethylphenyl)-2-imidazolidinylidene, 1,3-bis(2,4,6-trimethylphenyl) imidazol-2-ylidene, 1,3-bis(2,6-di-isopropylphenyl)-2-imidazolidinylidene, and 1,3-bis(2,6-di-isopropylphenyl)imidazol-2-ylidene;

X¹ and X² are chloride;

Y is oxygen;

R⁵, R⁶, R⁷, and R⁸ are each hydrogen;

n is 1; and

Z is isopropyl.

Suitable supports for any of the catalysts described herein may be of synthetic, semi-synthetic, or naturally occurring materials, which may be organic or inorganic, e.g., polymeric, ceramic, or metallic. Attachment to the support will generally, although not necessarily, be covalent, and the covalent linkage may be direct or indirect. Indirect covalent linkages are typically, though not necessarily, through a functional group on a support surface. Ionic attachments are also suitable, including combinations of one or more anionic groups on the metal complexes coupled with supports containing cationic groups, or combinations of one or more cationic groups on the metal complexes coupled with supports containing anionic groups.

When utilized, suitable supports may be selected from silicas, silicates, aluminas, aluminum oxides, silica-aluminas, aluminosilicates, zeolites, titanias, titanium dioxide, magnetite, magnesium oxides, boron oxides, clays, zirconias, zirconium dioxide, carbon, polymers, cellulose, cellulosic polymers amylose, amylosic polymers, or a combination thereof. The support preferably comprises silica, a silicate, or a combination thereof.

In certain embodiments, it is also possible to use a support that has been treated to include functional groups, inert moieties, and/or excess ligands. Any of the functional groups described herein are suitable for incorporation on the support, and may be generally accomplished through techniques known in the art. Inert moieties may also be incorporated on the support to generally reduce the available attachment sites on the support, e.g., in order to control the placement, or amount, of a complex linked to the support.

The metathesis catalysts that are described infra may be utilized in olefin metathesis reactions according to techniques known in the art. The catalyst is typically added to the resin composition as a solid, a solution, or as a suspension. When the catalyst is added to the resin composition as a suspension, the catalyst is suspended in a dispersing carrier such as mineral oil, paraffin oil, soybean oil, tri-isopropylbenzene, or any hydrophobic liquid which has a sufficiently high viscosity so as to permit effective dispersion of the catalyst, and which is sufficiently inert and which has a sufficiently high boiling point so that is does not act as a low-boiling impurity in the olefin metathesis reaction. It will be appreciated that the amount of catalyst that is used (i.e., the “catalyst loading”) in the reaction is dependent upon a variety of factors such as the identity of the reactants and the reaction conditions that are employed. It is therefore understood that catalyst loading may be optimally and independently chosen for each reaction. In general, however, the catalyst will be present in an amount that ranges from a low of about 0.1 ppm, 1 ppm, or 5 ppm, to a high of about 10 ppm, 15 ppm, 25 ppm, 50 ppm, 100 ppm, 200 ppm, 500 ppm, or 1000 ppm relative to the amount of an olefinic substrate.

The catalyst will generally be present in an amount that ranges from a low of about 0.00001 mol %, 0.0001 mol %, or 0.0005 mol %, to a high of about 0.001 mol %, 0.0015 mol %, 0.0025 mol %, 0.005 mol %, 0.01 mol %, 0.02 mol %, 0.05 mol %, or 0.1 mol % relative to the olefinic substrate.

When expressed as the molar ratio of monomer to catalyst, the catalyst (the “monomer to catalyst ratio”), loading will generally be present in an amount that ranges from a low of about 10,000,000:1, 1,000,000:1, or 200,00:1, to a high of about 100,000:1 66,667:1, 40,000:1, 20,000:1, 10,000:1, 5,000:1, or 1,000:1.

### Soluble Metal Salts

Soluble metal salts of this invention are of the formula:

M_{Ti}(OR¹)ₘₙ(OR²)ₘₘ and M_{Mg}[M_{Ti}(OR¹)ₘₙ(OR²)ₘₘ]ₘₒ,

where M_{Ti} is a metal selected from titanium, zirconium, hafnium, aluminum, gallium, indium, germanium, bismuth, and a lanthanide,

R¹ and R² are independently selected from hydrogen, hydrocarbyl, substituted hydrocarbyl, heteroatom-containing hydrocarbyl, substituted heteroatom-containing hydrocarbyl, and functional groups, or R¹ and R² can be taken together to form one or more cyclic groups, and further wherein R¹ and R² may be attached to a support,

mn, mm, and mo are integers that result in a net neutral charge for the metal salt, and

M_{Mg} is selective from Main Group Metals 3 and 4.

In a more preferred embodiment:

preferred M_{Ti} metals include titanium, zirconium, hafnium, aluminum, gallium, and indium,

R¹ and R² are independently selected from hydrogen, hydrocarbyl (e.g., C₁-C₂₀ alkyl, C₂-C₂₀ alkenyl, C₂-C₂₀ alkynyl, C₅-C₂₄ aryl, C₆-C₂₄ alkaryl, C₆-C₂₄ aralkyl, etc.), substituted hydrocarbyl (e.g., substituted C₁-C₂₀ alkyl, C₂-C₂₀ alkenyl, C₂-C₂₀ alkynyl, C₅-C₂₄ aryl, C₆-C₂₄ alkaryl, C₆-C₂₄ aralkyl, etc.), heteroatom-containing hydrocarbyl (e.g., heteroatom-containing C₁-C₂₀ alkyl, C₂-C₂₀ alkenyl, C₂-C₂₀ alkynyl, C₅-C₂₄ aryl, C₆-C₂₄ alkaryl, C₆-C₂₄ aralkyl, etc.), and substituted heteroatom-containing hydrocarbyl (e.g., substituted heteroatom-containing C₁-C₂₀ alkyl, C₂-C₂₀ alkenyl alkynyl, C₅-C₂₄, C₂-C₂₀ aryl, C₆-C₂₄ alkaryl, C₆-C₂₄ aralkyl, etc.), and functional groups. R¹ and R² may also be linked to form a cyclic group, which may be aliphatic or aromatic, and may contain substituents and/or heteroatoms. Generally, such a cyclic group will contain 4 to 12, preferably 5, 6, 7, or 8 ring atoms.

In preferred metal salts, R¹ and R² are independently selected from hydrogen, C₁-C₂₀ alkyl, C₂-C₂₀ alkenyl, and C₅-C₂₄ aryl, more preferably C₁-C₆ alkyl, C₂-C₆ alkenyl, and C₅-C₁₄ aryl.

More preferably, R¹ and R² are independently selected from methyl, ethyl, propyl, isopropyl, butyl, t-butyl, or phenyl, optionally the phenyl, substituted with one or more moieties selected from C₁-C₆ alkyl, halide, or heteroatom-containing C₁-C₂₀ alkyl, C₂-C₂₀ alkenyl, C₂-C₂₀ alkynyl, C₅-C₂₄ aryl, C₆-C₂₄ alkaryl, and C₆-C₂₄ aralkyl.

Mn, mm, and mo are integers independently selected from 0 to 8 which result in a net neutral charge for the metal salt, and

M_{Mg} is selective from beryllium, magnesium, calcium, strontium, barium, aluminum, gallium, and indium.

Even more preferred embodiment:

M_{Ti} metals are titanium and aluminum,

R¹ and R² are independently selective from methyl, ethyl, propyl, isopropyl, butyl, t-butyl, or phenyl,

mn and mm are integers and their sum is 3 or 4; and result in a net neutral charge for the metal salt, and

mo is an integer containing 2 or 3; and results in a net neutral charge for the metal salt.

The most preferred embodiment:

M_{Ti}(OR¹)ₘₙ(OR²)ₘₘ is Ti(O-isopropoxide)₄ and Al(O-isopropoxide)₃, and

M_{Mg}[M_{Ti}(OR¹)ₘₙ(OR²)ₘₘ]ₘₒ is Mg[Al(O-isopropoxide)₄]₂

Examples of soluble metal salts of the invention include, without limitation aluminum isopropoxide (Al(Oⁱ)O₃), magnesium aluminum isopropoxide (MgAl₂(OⁱPr)₈), titanium (IV) isopropoxide (Ti(OⁱPr)₄), titanium (IV) methoxide (Ti(Ome)₄), titanium (IV) butoxide (Ti(Obu)₄), titanium (IV) t-butoxide (Ti(OₜBu)₄), titanium (IV) ethoxide (Ti(Oet)₄), titanium (IV) 2-ethylhexyloxide (Ti(OCH₂CH(Et)(CH₂)₃CH₃)₄), titanium (iV) diisopropoxide bis(acetylacetonate), hafnium (IV) isopropoxide (Hf(OⁱPr)₄), hafnium (IV) butoxide (Hf(Obu)₄), hafnium (IV) t-butoxide (Hf(^{t}Obu)₄), zirconium (IV) isopropoxide (Zr(OⁱPr)₄), zirconium (IV) butoxide Zr(Obu)₄), zirconium (IV) t-butoxide (Zr(^{t}Obu)₄), zirconium (IV) ethoxide (Zr(Oet)₄), zirconium (IV) acetate hydroxide (Zr(Oac)ₓ(OH)_{y} x+y=4), methylaluminoxane (MAO), bismuth neodecanoate (Bi(Neodec)₃), and cerium acetate hydrate (Ce(Oac)₃ xH₂O).

Examples of preferred soluble metal salts of the invention include aluminum isopropoxide (Al(OiPr)₃), magnesium aluminum isopropoxide (MgAl₂(OiPr)₈), titanium (IV) isopropoxide (Ti(OiPr)₄), titanium (IV) butoxide (Ti(Obu)4), titanium (IV) t-butoxide (Ti(OtBu)4), titanium (IV) ethoxide (Ti(Oet)₄), titanium (IV) 2-ethylhexyloxide (Ti(OCH₂CH(Et)(CH₂)₃CH₃)₄), hafnium (IV) isopropoxide (Hf(OiPr)₄), hafnium (IV) t-butoxide (Hf(tOBu)4), zirconium (IV) isopropoxide (Zr(OiPr)₄), zirconium (IV) butoxide Zr(Obu)₄), zirconium (IV) t-butoxide (Zr(^{t}Obu)₄), zirconium (IV) ethoxide (Zr(Oet)₄), and methylaluminoxane (MAO).

Most preferred soluble metal salts a include include aluminum isopropoxide (Al(OiPr)₃), magnesium aluminum isopropoxide (MgAl₂(OiPr)₈), titanium (IV) isopropoxide (Ti(OiPr)₄), and methylaluminoxane (MAO).

### Cyclic Olefin

Resin compositions that may be used with the present invention disclosed herein comprise one or more cyclic olefins. In general, any cyclic olefin suitable for the metathesis reactions disclosed herein may be used. Such cyclic olefins may be optionally substituted, optionally heteroatom-containing, mono-unsaturated, di-unsaturated, or poly-unsaturated C₅ to C₂₄ hydrocarbons that may be mono-, di-, or poly-cyclic. The cyclic olefin may generally be any strained or unstrained cyclic olefin, provided the cyclic olefin is able to participate in a ROMP reaction either individually or as part of a ROMP cyclic olefin composition. While certain unstrained cyclic olefins such as cyclohexene are generally understood to not undergo ROMP reactions by themselves, under appropriate circumstances, such unstrained cyclic olefins may nonetheless be ROMP active. For example, when present as a co-monomer in a ROMP composition, unstrained cyclic olefins may be ROMP active. Accordingly, as used herein and as would be appreciated by the skilled artisan, the term “unstrained cyclic olefin” is intended to refer to those unstrained cyclic olefins that may undergo a ROMP reaction under any conditions, or in any ROMP composition, provided the unstrained cyclic olefin is ROMP active.

In general, the cyclic olefin may be represented by the structure of formula (A)

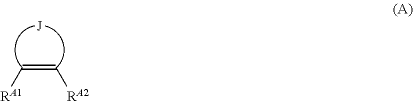

wherein J, R^{A1}, and R^{A2} are as follows:

R^{A1} and R^{A2} is selected independently from the group consisting of hydrogen, hydrocarbyl (e.g., C₁-C₂₀ alkyl, C₅-C₂₀ aryl, C₅-C₃₀ aralkyl, or C₅-C₃₀ alkaryl), substituted hydrocarbyl (e.g., substituted C₁-C₂₀ alkyl, C₅-C₂₀ aryl, C₅-C₃₀ aralkyl, or C₅-C₃₀ alkaryl), heteroatom-containing hydrocarbyl (e.g., C₁-C₂₀ heteroalkyl, C₅-C₂₀ heteroaryl, heteroatom-containing C₅-C₃₀ aralkyl, or heteroatom-containing C₅-C₃₀ alkaryl), and substituted heteroatom-containing hydrocarbyl (e.g., substituted C₁-C₂₀ heteroalkyl, C₅-C₂₀ heteroaryl, heteroatom-containing C₅-C₃₀ aralkyl, or heteroatom-containing C₅-C₃₀ alkaryl) and, if substituted hydrocarbyl or substituted heteroatom-containing hydrocarbyl, wherein the substituents may be functional groups (“Fn”) such as phosphonato, phosphoryl, phosphanyl, phosphino, sulfonato, C₁-C₂₀ alkylsulfanyl, C₅-C₂₀ arylsulfanyl, C₁-C₂₀ alkylsulfonyl, C₅-C₂₀ arylsulfonyl, C₁-C₂₀ alkylsulfinyl, C₅-C₂₀ arylsulfinyl, sulfonamido, amino, amido, imino, nitro, nitroso, hydroxyl, C₁-C₂₀ alkoxy, C₅-C₂₀ aryloxy, C₂-C₂₀ alkoxycarbonyl, C₅-C₂₀ aryloxycarbonyl, carboxyl, carboxylato, mercapto, formyl, C₁-C₂₀ thioester, cyano, cyanato, thiocyanato, isocyanate, thioisocyanate, carbamoyl, epoxy, styrenyl, silyl, silyloxy, silanyl, siloxazanyl, boronato, boryl, or halogen, or a metal-containing or metalloid-containing group (wherein the metal may be, for example, Sn or Ge). R^{A1} and R^{A2} may itself be one of the aforementioned groups, such that the Fn moiety is directly bound to the olefinic carbon atom indicated in the structure. In the latter case, however, the functional group will generally not be directly bound to the olefinic carbon through a heteroatom containing one or more lone pairs of electrons, e.g., an oxygen, sulfur, nitrogen, or phosphorus atom, or through an electron-rich metal or metalloid such as Ge, Sn, As, Sb, Se, Te, etc. With such functional groups, there will normally be an intervening linkage Z*, such that R^{A1} and/or R^{A2} then has the structure —(Z*)ₙ—Fn wherein n is 1, Fn is the functional group, and Z* is a hydrocarbylene linking group such as an alkylene, substituted alkylene, heteroalkylene, substituted heteroalkene, arylene, substituted arylene, heteroarylene, or substituted heteroarylene linkage.

J is a saturated or unsaturated hydrocarbylene, substituted hydrocarbylene, heteroatom-containing hydrocarbylene, or substituted heteroatom-containing hydrocarbylene linkage, wherein when J is substituted hydrocarbylene or substituted heteroatom-containing hydrocarbylene, the substituents may include one or more —(Z*)ₙ-Fn groups, wherein n is zero or 1, and Fn and Z* are as defined previously. Additionally, two or more substituents attached to ring carbon (or other) atoms within J may be linked to form a bicyclic or polycyclic olefin. J will generally contain in the range of approximately 5 to 14 ring atoms, typically 5 to 8 ring atoms, for a monocyclic olefin, and, for bicyclic and polycyclic olefins, each ring will generally contain 4 to 8, typically 5 to 7, ring atoms.

Mono-unsaturated cyclic olefins encompassed by structure (A) may be represented by the structure (B)

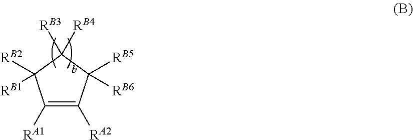

wherein b is an integer generally although not necessarily in the range of 1 to 10, typically 1 to 5.

R^{A1} and R^{A2} are as defined above for structure (A), and R^{B1}, R^{B2}, R^{B3}, R^{B4}, R^{B5}, and R^{B6} are independently selected from the group consisting of hydrogen, hydrocarbyl, substituted hydrocarbyl, heteroatom-containing hydrocarbyl, substituted heteroatom-containing hydrocarbyl and —(Z*)ₙ-Fn where n, Z* and Fn are as defined previously, and wherein if any of the R^{B1} through R^{B6} moieties is substituted hydrocarbyl or substituted heteroatom-containing hydrocarbyl, the substituents may include one or more —(Z*)ₙ-Fn groups. Accordingly, R^{B1}, R^{B2}, R^{B3}, R^{B4}, R^{B5}, and R^{B6} may be, for example, hydrogen, hydroxyl, C₁-C₂₀ alkyl, C₅-C₂₀ aryl, C₁-C₂₀ alkoxy, C₅-C₂₀ aryloxy, C₂-C₂₀ alkoxycarbonyl, C₅-C₂₀ aryloxycarbonyl, amino, amido, nitro, etc. Furthermore, any of the R^{B1}, R^{B2}, R^{B3}, R^{B4}, R^{B5}, and R^{B6} moieties can be linked to any of the other R^{B1}, R^{B3}, R^{B4}, R^{B5}, and R^{B6} moieties to provide a substituted or unsubstituted alicyclic group containing 4 to 30 ring carbon atoms or a substituted or unsubstituted aryl group containing 6 to 18 ring carbon atoms or combinations thereof and the linkage may include heteroatoms or functional groups, e.g., the linkage may include without limitation an ether, ester, thioether, amino, alkylamino, imino, or anhydride moiety. The alicyclic group can be monocyclic, bicyclic, or polycyclic. When unsaturated the cyclic group can contain monounsaturation or multiunsaturation, with monounsaturated cyclic groups being preferred. When substituted, the rings contain monosubstitution or multisubstitution wherein the substituents are independently selected from hydrogen, hydrocarbyl, substituted hydrocarbyl, heteroatom-containing hydrocarbyl, substituted heteroatom-containing hydrocarbyl, —(Z*)ₙ-Fn where n is zero or 1, Z* and Fn are as defined previously, and functional groups (Fn) provided above.

Examples of monounsaturated, monocyclic olefins encompassed by structure (B) include, without limitation, cyclopentene, cyclohexene, cycloheptene, cyclooctene, cyclononene, cyclodecene, cycloundecene, cyclododecene, tricyclodecene, tetracyclodecene, octacyclodecene, and cycloeicosene, and substituted versions thereof such as 1-methylcyclopentene, 1-ethylcyclopentene, 1-isopropylcyclohexene, 1-chloropentene, 1-fluorocyclopentene, 4-methylcyclopentene, 4-methoxy-cyclopentene, 4-ethoxy-cyclopentene, cyclopent-3-ene-thiol, cyclopent-3-ene, 4-methylsulfanyl-cyclopentene, 3-methylcyclohexene, 1-methylcyclooctene, 1,5-dimethylcyclooctene, etc.

Monocyclic diene reactants encompassed by structure (A) may be generally represented by the structure (C)

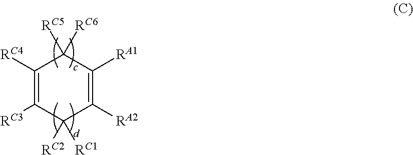

wherein c and d are independently integers in the range of 1 to about 8, typically 2 to 4, preferably 2 (such that the reactant is a cyclooctadiene), R^{A1} and R^{A2} are as defined above for structure (A), and R^{C1}, R^{C2}, R^{C3}, R^{C4}, R^{C5}, and R^{C6} are defined as for RB¹ through R^{B6}. In this case, it is preferred that R^{C3} and R^{C4} be non-hydrogen substituents, in which case the second olefinic moiety is tetrasubstituted. Examples of monocyclic diene reactants include, without limitation, 1,3-cyclopentadiene, 1,3-cyclohexadiene, 1,4-cyclohexadiene, 5-ethyl-1,3-cyclohexadiene, 1,3-cycloheptadiene, cyclohexadiene, 1,5-cyclooctadiene, 1,3-cyclooctadiene, and substituted analogs thereof. Triene reactants are analogous to the diene structure (C), and will generally contain at least one methylene linkage between any two olefinic segments.

Bicyclic and polycyclic olefins encompassed by structure (A) may be generally represented by the structure (D)

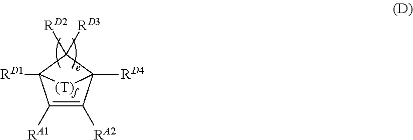

wherein R^{A1} and R^{A2} are as defined above for structure (A), R^{D1}, R^{D2}, R^{D3}, and R^{D4} are as defined for R^{B1} through R^{B6}, e is an integer in the range of 1 to 8 (typically 2 to 4) f is generally 1 or 2; T is lower alkylene or alkenylene (generally substituted or unsubstituted methyl or ethyl), CHR^{G1}, C(R^{G1})₂, O, S, N—R^{G1}, P—R^{G1}, O═P—R^{G1}, Si(R^{G1})₂, B—R^{G1}, or As—R^{G1} where R^{G1} is alkyl, alkenyl, cycloalkyl, cycloalkenyl, aryl, alkaryl, aralkyl, or alkoxy. Furthermore, any of the R^{D1}, R^{D2}, R^{D3}, and R^{D4} moieties can be linked to any of the other R^{D1}, R^{D2}, R^{D3}, and R^{D4} moieties to provide a substituted or unsubstituted alicyclic group containing 4 to 30 ring carbon atoms or a substituted or unsubstituted aryl group containing 6 to 18 ring carbon atoms or combinations thereof and the linkage may include heteroatoms or functional groups, e.g., the linkage may include without limitation an ether, ester, thioether, amino, alkylamino, imino, or anhydride moiety. The cyclic group can be monocyclic, bicyclic, or polycyclic. When unsaturated the cyclic group can contain monounsaturation or multiunsaturation, with monounsaturated cyclic groups being preferred. When substituted, the rings contain monosubstitution or multisubstitution wherein the substituents are independently selected from hydrogen, hydrocarbyl, substituted hydrocarbyl, heteroatom-containing hydrocarbyl, substituted heteroatom-containing hydrocarbyl, —(Z*)ₙ-Fn where n is zero or 1, Z* and Fn are as defined previously, and functional groups (Fn) provided above.

Cyclic olefins encompassed by structure (D) are in the norbornene family. As used herein, norbornene means any compound that includes at least one norbornene or substituted norbornene moiety, including without limitation norbornene, substituted norbornene(s), norbornadiene, substituted norbornadiene(s), polycyclic norbornenes, and substituted polycyclic norbornene(s). Norbornenes within this group may be generally represented by the structure (E)

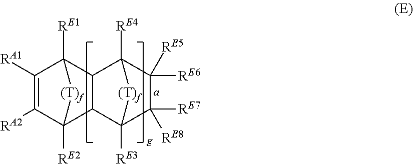

wherein R^{A} and R^{A2} are as defined above for structure (A), T is as defined above for structure (D), R^{E1}, R^{E2}, R^{E3}, R^{E4}, R^{E5}, R^{E6}, R^{E7}, and R^{E8} are as defined for R^{B1} through R^{B6}, and “a” represents a single bond or a double bond, f is generally 1 or 2, “g” is an integer from 0 to 5, and when “a” is a double bond one of R^{E5}, R^{E6} and one of R^{E7}, R^{E8} is not present.

Furthermore, any of the R^{E5}, R^{E6}, R^{E7}, and R^{E8} moieties can be linked to any of the other R^{E5}, R^{E6}, R^{E7}, and R^{E8} moieties to provide a substituted or unsubstituted alicyclic group containing 4 to 30 ring carbon atoms or a substituted or unsubstituted aryl group containing 6 to 18 ring carbon atoms or combinations thereof and the linkage may include heteroatoms or functional groups, e.g., the linkage may include without limitation an ether, ester, thioether, amino, alkylamino, imino, or anhydride moiety. The cyclic group can be monocyclic, bicyclic, or polycyclic. When unsaturated the cyclic group can contain monounsaturation or multiunsaturation, with monounsaturated cyclic groups being preferred. When substituted, the rings contain monosubstitution or multisubstitution wherein the substituents are independently selected from hydrogen, hydrocarbyl, substituted hydrocarbyl, heteroatom-containing hydrocarbyl, substituted heteroatom-containing hydrocarbyl, —(Z*)ₙ-Fn where n is zero or 1, Z* and Fn are as defined previously, and functional groups (Fn) provided above.

More preferred cyclic olefins possessing at least one norbornene moiety have the structure (F):

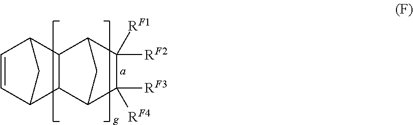

wherein, R^{F1}, R^{F2}, R^{F3}, and R^{F4}, are as defined for R^{B1} through R^{B6}, and “a” represents a single bond or a double bond, “g” is an integer from 0 to 5, and when “a” is a double bond one of R^{F1}, R^{F2} and one of R^{F3}, R^{F4} is not present.

Furthermore, any of the R^{F1}, R^{F2}, R^{F3}, and R^{F4} moieties can be linked to any of the other R^{F1}, R^{F2}, R^{F3}, and R^{F4} moieties to provide a substituted or unsubstituted alicyclic group containing 4 to 30 ring carbon atoms or a substituted or unsubstituted aryl group containing 6 to 18 ring carbon atoms or combinations thereof and the linkage may include heteroatoms or functional groups, e.g., the linkage may include without limitation an ether, ester, thioether, amino, alkylamino, imino, or anhydride moiety. The alicyclic group can be monocyclic, bicyclic, or polycyclic. When unsaturated the cyclic group can contain monounsaturation or multiunsaturation, with monounsaturated cyclic groups being preferred. When substituted, the rings contain monosubstitution or multisubstitution wherein the substituents are independently selected from hydrogen, hydrocarbyl, substituted hydrocarbyl, heteroatom-containing hydrocarbyl, substituted heteroatom-containing hydrocarbyl, —(Z*)ₙ-Fn where n is zero or 1, Z* and Fn are as defined previously, and functional groups (Fn) provided above.

Examples of bicyclic and polycyclic olefins thus include, without limitation, dicyclopentadiene (DCPD); trimer and other higher order oligomers of cyclopentadiene including without limitation tricyclopentadiene (cyclopentadiene trimer), cyclopentadiene tetramer, and cyclopentadiene pentamer; ethylidenenorbornene; dicyclohexadiene; norbornene; 5-methyl-2-norbornene; 5-ethyl-2-norbornene; 5-isobutyl-2-norbornene; 5,6-dimethyl-2-norbornene; 5-phenylnorbornene; 5-benzylnorbornene; 5-acetylnorbornene; 5-methoxycarbonylnorbornene; 5-ethyoxycarbonyl-1-norbornene; 5-methyl-5-methoxy-carbonylnorbornene; 5-cyanonorbornene; 5,5,6-trimethyl-2-norbornene; cyclo-hexenylnorbornene; endo, exo-5,6-dimethoxynorbornene; endo, endo-5,6-dimethoxynorbornene; endo, exo-5,6-dimethoxycarbonylnorbornene; endo,endo-5,6-dimethoxycarbonylnorbornene; 2,3-dimethoxynorbornene; norbornadiene; tricycloundecene; tetracyclododecene; 8-methyltetracyclododecene; 8-ethyltetracyclododecene; 8-methoxycarbonyltetracyclododecene; 8-methyl-8-tetracyclododecene; 8-cyanotetracyclododecene; pentacyclopentadecene; pentacyclohexadecene; and the like, and their structural isomers, stereoisomers, and mixtures thereof. Additional examples of bicyclic and polycyclic olefins include, without limitation, C₂-C₁₂ hydrocarbyl substituted norbornenes such as 5-butyl-2-norbornene, 5-hexyl-2-norbornene, 5-octyl-2-norbornene, 5-decyl-2-norbornene, 5-dodecyl-2-norbornene, 5-vinyl-2-norbornene, 5-ethylidene-2-norbornene, 5-isopropenyl-2-norbornene, 5-propenyl-2-norbornene, and 5-butenyl-2-norbornene, and the like.

Preferred cyclic olefins include C₅ to C₂₄ unsaturated hydrocarbons. Also preferred are C₅ to C₂₄ cyclic hydrocarbons that contain one or more (typically 2 to 12) heteroatoms such as O, N, S, or P. For example, crown ether cyclic olefins may include numerous 0 heteroatoms throughout the cycle, and these are within the scope of the invention. In addition, preferred cyclic olefins are C₅ to C₂₄ hydrocarbons that contain one or more (typically 2 or 3) olefins. For example, the cyclic olefin may be mono-, di-, or tri-unsaturated. Examples of cyclic olefins include without limitation cyclooctene, cyclododecene, and (c,t,t)-1,5,9-cyclododecatriene.

The cyclic olefins may also comprise multiple (typically 2 or 3) rings. For example, the cyclic olefin may be mono-, di-, or tri-cyclic. When the cyclic olefin comprises more than one ring, the rings may or may not be fused. Preferred examples of cyclic olefins that comprise multiple rings include norbornene, dicyclopentadiene, tricyclopentadiene, and 5-ethylidene-2-norbornene.

The cyclic olefin may also be substituted, for example, a C₅ to C₂₄ cyclic hydrocarbon wherein one or more (typically 2, 3, 4, or 5) of the hydrogens are replaced with non-hydrogen substituents. Suitable non-hydrogen substituents may be chosen from the substituents described hereinabove. For example, functionalized cyclic olefins, i.e., C₅ to C₂₄ cyclic hydrocarbons wherein one or more (typically 2, 3, 4, or 5) of the hydrogens are replaced with functional groups, are within the scope of the invention. Suitable functional groups may be chosen from the functional groups described hereinabove. For example, a cyclic olefin functionalized with an alcohol group may be used to prepare a telechelic polymer comprising pendent alcohol groups. Functional groups on the cyclic olefin may be protected in cases where the functional group interferes with the metathesis catalyst, and any of the protecting groups commonly used in the art may be employed. Acceptable protecting groups may be found, for example, in Greene et al., Protective Groups in Organic Synthesis, 3rd Ed. (New York: Wiley, 1999). Examples of functionalized cyclic olefins include without limitation 2-hydroxymethyl-5-norbornene, 2-[(2-hydroxyethyl)carboxylate]-5-norbornene, cydecanol, 5-n-hexyl-2-norbornene, 5-n-butyl-2-norbornene.

Cyclic olefins incorporating any combination of the abovementioned features (i.e., heteroatoms, substituents, multiple olefins, multiple rings) are suitable for the methods disclosed herein. Additionally, cyclic olefins incorporating any combination of the abovementioned features (i.e., heteroatoms, substituents, multiple olefins, multiple rings) are suitable for the invention disclosed herein.

The cyclic olefins useful in the methods disclosed herein may be strained or unstrained. It will be appreciated that the amount of ring strain varies for each cyclic olefin compound, and depends upon a number of factors including the size of the ring, the presence and identity of substituents, and the presence of multiple rings. Ring strain is one factor in determining the reactivity of a molecule towards ring-opening olefin metathesis reactions. Highly strained cyclic olefins, such as certain bicyclic compounds, readily undergo ring opening reactions with olefin metathesis catalysts. Less strained cyclic olefins, such as certain unsubstituted hydrocarbon monocyclic olefins, are generally less reactive. In some cases, ring opening reactions of relatively unstrained (and therefore relatively unreactive) cyclic olefins may become possible when performed in the presence of the olefinic compounds disclosed herein. Additionally, cyclic olefins useful in the invention disclosed herein may be strained or unstrained.

The resin compositions of the present invention may comprise a plurality of cyclic olefins. A plurality of cyclic olefins may be used to prepare metathesis polymers from the olefinic compound. For example, two cyclic olefins selected from the cyclic olefins described hereinabove may be employed in order to form metathesis products that incorporate both cyclic olefins. Where two or more cyclic olefins are used, one example of a second cyclic olefin is a cyclic alkenol, i.e., a C₅-C₂₄ cyclic hydrocarbon wherein at least one of the hydrogen substituents is replaced with an alcohol or protected alcohol moiety to yield a functionalized cyclic olefin.

The use of a plurality of cyclic olefins, and in particular when at least one of the cyclic olefins is functionalized, allows for further control over the positioning of functional groups within the products. For example, the density of cross-linking points can be controlled in polymers and macromonomers prepared using the methods disclosed herein. Control over the quantity and density of substituents and functional groups also allows for control over the physical properties (e.g., melting point, tensile strength, glass transition temperature, etc.) of the products. Control over these and other properties is possible for reactions using only a single cyclic olefin, but it will be appreciated that the use of a plurality of cyclic olefins further enhances the range of possible metathesis products and polymers formed.

More preferred cyclic olefins include dicyclopentadiene; tricyclopentadiene; dicyclohexadiene; norbornene; 5-methyl-2-norbornene; 5-ethyl-2-norbornene; 5-isobutyl-2-norbornene; 5,6-dimethyl-2-norbornene; 5-phenylnorbornene; 5-benzylnorbornene; 5-acetylnorbornene; 5-methoxycarbonylnorbornene; 5-ethoxycarbonyl-1-norbornene; 5-methyl-5-methoxy-carbonylnorbornene; 5-cyanonorbornene; 5,5,6-trimethyl-2-norbornene; cyclo-hexenylnorbornene; endo, exo-5,6-dimethoxynorbornene; endo, endo-5,6-dimethoxynorbornene; endo, exo-5-6-dimethoxycarbonylnorbornene; endo, endo-5,6-dimethoxycarbonylnorbornene; 2,3-dimethoxynorbornene; norbornadiene; tricycloundecene; tetracyclododecene; 8-methyltetracyclododecene; 8-ethyl-tetracyclododecene; 8-methoxycarbonyltetracyclododecene; 8-methyl-8-tetracyclo-dodecene; 8-cyanotetracyclododecene; pentacyclopentadecene; pentacyclohexadecene; higher order oligomers of cyclopentadiene such as cyclopentadiene tetramer, cyclopentadiene pentamer, and the like; and C₂-C₁₂ hydrocarbyl substituted norbornenes such as 5-butyl-2-norbornene; 5-hexyl-2-norbornene; 5-octyl-2-norbornene; 5-decyl-2-norbornene; 5-dodecyl-2-norbornene; 5-vinyl-2-norbornene; 5-ethylidene-2-norbornene; 5-isopropenyl-2-norbornene; 5-propenyl-2-norbornene; and 5-butenyl-2-norbornene, and the like. Even more preferred cyclic olefins include dicyclopentadiene, tricyclopentadiene, and higher order oligomers of cyclopentadiene, such as cyclopentadiene tetramer, cyclopentadiene pentamer, and the like, tetracyclododecene, norbornene, and C₂-C₁₂ hydrocarbyl substituted norbornenes, such as 5-butyl-2-norbornene, 5-hexyl-2-norbornene, 5-octyl-2-norbornene, 5-decyl-2-norbornene, 5-dodecyl-2-norbornene, 5-vinyl-2-norbornene, 5-ethylidene-2-norbornene, 5-isopropenyl-2-norbornene, 5-propenyl-2-norbornene, 5-butenyl-2-norbornene, and the like.

### Materials and Methods

All solvents and reagents were purchase from commercial suppliers and used as received unless otherwise noted. All reactions were performed under ambient conditions unless otherwise noted. Seed oil used in this study included Salad Grade Soybean Oil obtained from Costco, Clear Valley® Canola Oil and Trans Advantage® Palm Oil were obtained from Cargill, purified Camelina Oil was obtained from POS Bio-Sciences (Saskatoon, SK, Canada), and Methyl Oleate was obtained from NuCheck Prep Inc. (Elysian, Minn.).

1-Hexene, 1-decene, cyclooctadiene (COD), 1-dodecene, N-Boc-diallylamine, titanium (IV) isopropoxide (Ti(OⁱPr)₄), methylaluminoxane (MAO) 10 wt % in toluene, alumina (Al₂O₃) neutral, basic and acidic Brockmann I activated standard grades, sodium borohydride (NaBH₄), bismuth neodecanoate (Bi(Neodec)₃), and cerium acetate hydrate (Ce(OAc)₃ xH₂O) were obtained from Aldrich. Aluminum isopropoxide (Al(OⁱPr)₃), magnesium aluminum isopropoxide (MgAl₂(OⁱPr)₈), and magnetite (Fe₃O₄) were purchased from Strem Chemicals, Inc. (Newburyport, Mass.).

5-Decene was made by the self-metathesis of 1-hexene as described in U.S. Pat. No. 6,215,019, 9-octadecene was made by an analogous procedure expect using 1-decene. Methyl-9-dodecenoate was prepared as described in Topics in Catalysis 2012, 55, 518 and tris-hydroxymethylphosphine (THMP) was prepared as described in Adv. Synth. Catalysis 2002, 344, 728.

Ultrene® 99 was obtained from Cymetech Corp. Ultrene® 99 is 99% purity dicyclopentadiene (DCPD). Prometa® 2100 was obtained from Materia Inc., Prometa® 2100 is Ultrene® 99 containing 6% tricyclopentadiene (trimer). Trimer was prepared by heat cracking Ultrene® 99 as described in U.S. Pat. No. 4,899,005.

Catalysts [1,3-Bis-(2,4,6-trimethylphenyl)-2-imidazolidinylidene]dichloro(o-isopropoxyphenylmethylene)ruthenium (C627), ruthenium (II) dichloro[1,3-bis(2,4,6-trimethylphenyl)-2-imidazolidinylidene](3-methyl-1,2-butenylidene)bis(pyridine) (C705), [1,3-bis-(2,4,6-trimethylphenyl)-2-imidazolidinylidene]dichloro(3-methyl-2-butenylidene)(tricyclohexylphosphine) ruthenium (II) (C827), [1,3-Bis(2,4,6-trimethylphenyl)-2-imidazolidinylidene]dichloro(phenylmethylene)(triphenylphosphine) ruthenium (II) (C831), and [1,3-Bis(2,4,6-trimethylphenyl)-2-imidazolidinylidene]dichloro(phenylmethylene)(tricyclohexylphosphine)ruthenium (II) (C848) were prepared by procedures described in U.S. Pat. Nos. 6,921,735; 6,759,537; 5,969,170; 7,329,758; 6,111,121; and 7,329,758.

### Soluble Metal Treatment Procedures

A general procedure for treating olefin metathesis substrates with soluble metal complexes involves adding the appropriate amount of soluble metal complex to the degassed metathesis substrate, in a glove box. The samples were allowed to age in the glove box (i.e., remain in the glove box at room temp) or were removed from the glove box and warmed in an oil bath. The soluble metal complexes were not removed from the metathesis substrates prior to metathesis. The soluble metal salt is usually mixed with the olefin and stirred from 2 to 96 hours. The mixture may be heated slightly above room temperature, i.e., 30° C., or up to 80° C. The loading of the soluble metal salt may be from 0.1 wt % to 5 wt %, with preferred loadings of 0.5 wt % to 2.0 wt %. The soluble metal salt may be added directly to the olefin or as a solution in another solvent such as toluene.

### Metathesis Reactions

Metathesis reactions were generally conducted in the glove box; to a 20 mL scintillation vial containing a septum cap and a Teflon-coated flee stir bar was added 5 g of the soluble metal complex treated metathesis substrate. The catalyst solution was added via syringe, the reactions were heated with stirring to the desired reaction temperature for 2 to 4 hours.

For alkenolysis reactions, the metathesis substrate and α-olefin were placed in a 20 mL scintillation vial containing a septum cap and a Teflon-coated flee stir bar were heated with stirring to the appropriate temperature, and the catalyst was added via syringe. The catalyst solutions were prepared in volumetric flasks inside a glove box.

Reaction conversion was monitored by GC analysis. Typical procedure, an aliquot of a metathesis reaction was removed from the metathesis reaction at the desired times (usually at 2 hours and 4 hours). Triglyceride containing substrates were added to 1% NaOMe/MeOH solution heated to 60° C. in a 20 mL scintillation vial for 1 hour. The mixture was cooled to room temperature and water, brine, and hexane were added to the sample. After phase separation, the organic phase was analyzed by GC using the GC method described above.

For non-triglyceride substrates, an aliquot of the metathesis reaction was removed from the metathesis reaction at the desired times and treated with THMP as described in Adv. Synth. Catalysis 2002, 344, 728, dried over anhydrous sodium sulfate, filtered and analyzed by GC.

### Alumina Treatment

Alumina was activated by heating in a 130° C. drying oven, exposed to air, for 24 h then cooled to room temp. The activated alumina was added to the metathesis substrates and aged in the glove box. The metathesis substrates were decanted from the alumina when needed in a metathesis reaction. It is important to avoid getting alumina dust in the metathesis reactions as this will adsorb the metathesis catalyst and inhibit the metathesis reaction.

### Peroxide Value (PV) Analysis

PV determination procedure is as reported AOCS Test Method, method can be found LUBRIZOL STANDARD TEST PROCEDURE AATM-51601, with the exception used isooctane in place of chloroform. PV is reported in ppm (meq/Kg).

### GC Analytical Methods

Volatile products were analyzed using an Agilent 6850 gas chromatography (GC) instrument with a flame ionization detector (FID). The following conditions and equipment were used:

Column: HP-5, 30 m×0.25 mm (ID)×0.25 μm film thickness.
Manufacturer: Agilent

GC and column conditions: Injector temperature: 250° C.
Detector temperature: 280° C.

Oven temperature: Starting temperature: 100° C., hold time: 1 minute.
Ramp rate 10° C./min to 250° C., hold time: 12 minutes.
Carrier gas: Helium

Mean gas velocity: 31.3±3.5% cm/sec (calculated)

Split ratio: ˜50:1

The products were characterized by comparing peaks with known standards, in conjunction with supporting data from mass spectrum analysis (GCMS-Agilent 5973N). GCMS analysis was accomplished with a second HP-5, 30 m×0.25 mm (ID)×0.25 μm film thickness GC column, using the same method as above.

### Experimental Reactions

### Reaction 1. Self Metathesis of Methyl Oleate

To test the effectiveness of the soluble metal salts, methyl oleate was purified using different purification conditions. In an inert atmosphere glove box, methyl oleate (5 g) samples were stirred with no additive (the control) (1a), 1 wt % alumina (1b) [Methyl oleate was treated with 1 wt % activated alumina (Brockman I neutral) for 3 days at room temp. A 5 g aliquot was removed for the metathesis reaction.], 1 wt % NaBH₄ (1c), 1 wt % Al(OⁱPr)₃ (1d), or 1 wt % MgAl₂(OⁱPr)₈ (1e) for 2 h at 50° C. The alumina and NaBH₄ treated samples were filtered prior to the metathesis reaction. The Al(OⁱPr)₃ and MgAl₂(OⁱPr)₈ treated samples were used in the metathesis reaction as is, GC results are reported in Table 1. Reactions 1d and 1e were treated with THMP prior to GC analysis.

The self-metathesis yields were measured by GC and represented in Table 1. The self-metathesis of untreated methyl oleate (1a) did not yield any product, while Al₂O₃ (1b) and NaBH₄ (1c) treated methyl oleates yielded only 20% and 6% products, respectively. The soluble metal salts (1d and 1e) reactions reached equilibrium after 30 minutes.

TABLE 1
Self-Metathesis of Methyl Oleate

| | Methyl Oleate | 9C₁₈/Diester^{a,b} |
|---|---|---|
| Reaction | Treatment | (GC area %) |
| 1a | None | <1 |
| 1b | Al₂O₃ | 20 |
| 1c | NaBH₄ | 6 |
| 1d | Al(OⁱPr)₃ | 49 |
| 1e | MgAl₂(OⁱPr)₈ | 49 |

^{a}Equilibrium is reached at 50% 9C₁₈/Diester
^{b}9C₁₈ is 9-octadecene and Diester is 1, 18-dimethyl ester of 9-octadecene

### Reaction 2. Peroxide Value Determination of Treated Soybean Oil (SBO)

Olefin metathesis catalysts are sensitive to peroxides in the olefinic feedstocks; to that end, soluble metal salts of the invention were added to the olefinic feedstocks to reduce peroxides and improve metathesis efficiencies.

SBO was treated with 0.1 wt % Al(OⁱPr)₃, 0.1 wt % MgAl₂(OⁱPr)₈, or 0.5 wt % MgAl₂(OⁱPr)₈ for four days at either 30° C. or 80° C. The PV of the untreated and treated samples were measured (Table 2). Samples treated with 0.1 wt % Al(OⁱPr)₃ and 0.1 wt % MgAl₂(OⁱPr)₈ had reduced PV to half their starting values after 4 days at 30° C. (2b and 2d). Heating to 80° C. was more effective at reducing PV (2c and 2e). SBO treated with 0.5 wt % MgAl₂(OⁱPr)₈ reduced PV to near zero, independent of treatment temperature (2f and 2g). PV was reduced to 1 by the addition of 0.5 wt % MgAl₂(OiPr)₈ and heated to at least 30° C. for 4 days.

TABLE 2
PV Determination of Treated SBO

| Reaction | Oil Treatment | Amount | Treatment Conditions | PV |
|---|---|---|---|---|
| 2a | None | — | — | 59 |
| 2b | Al(OⁱPr)₃ | 0.1 wt % | 4 days @ 30° C. | 52 |
| 2c | Al(OⁱPr)₃ | 0.1 wt % | 4 days @ 80° C. | 24 |
| 2d | MgAl₂(OⁱPr)₈ | 0.1 wt % | 4 days @ 30° C. | 45 |
| 2e | MgAl₂(OⁱPr)₈ | 0.1 wt % | 4 days @ 80° C. | 24 |
| 2f | MgAl₂(OⁱPr)₈ | 0.5 wt % | 4 days @ 30° C. | 1 |
| 2g | MgAl₂(OⁱPr)₈ | 0.5 wt % | 4 days @ 80° C. | 1 |

### Reaction 3. PV Determination of MgAl₂(OⁱPr)₈ Treated SBO

SBO was treated with 0.1 wt %, 0.2 wt %, or 0.5 wt % MgAl₂(OⁱPr)₈ at 80° C. for two days. The PV of the untreated and treated SBO samples are reported in Table 3. While SBO samples treated with 0.1 wt % or 0.2 wt % MgAl₂(OⁱPr)₈ had reduced the peroxide values to about half their original values (3b and 3c), the sample treated with 0.5 wt % MgAl₂(OⁱPr)₈ reduced the PV to 1 (3d).

TABLE 3
PV Determination of MgAl₂(OⁱPr)₈ Treated SBO

| Reaction | Oil Treatment | Treatment Conditions | PV |
|---|---|---|---|
| 3a | None | — | 46 |
| 3b | 0.1 wt % MgAl₂(OⁱPr)₈ | 2 days @ 80° C. | 24 |
| 3c | 0.2 wt % MgAl₂(OⁱPr)₈ | 2 days @ 80° C. | 19 |
| 3d | 0.5 wt % MgAl₂(OⁱPr)₈ | 2 days @ 80° C. | 1 |

### Reaction 4. PV Determination of Ce(OAc)₃, Fe₃O₄, Bi(Neodecanoate)₃, and Methylalumoxane (MAO) Treated SBO

SBO was treated with 1 wt % of Ce(OAc)₃, Fe₃O₄, Bi(Neodecanoate)₃, and MAO at 30° C. and 80° C. for 2 days. Ce(OAc)₃, Bi(Neodecanoate)₃, and MAO are soluble metal salt complexes, while Fe₃O₄ was an insoluble solid. The PV determinations of the untreated and treated samples were reported in Table 4. MAO was the most effective at reducing PV to <2 (4h and 4i), Ce(OAc)₃ and Fe₃O₄ treated samples were ineffective at 30° C. (4 b and 4 d). Interestingly Bi(neodecanoate)₃ treated SBO resulted in higher PVs.

TABLE 4
PV Determination of Ce(OAc)₃, Fe₃O₄,
Bi(Neodecanoate)₃, and MAO treated SBO

| Reaction | Oil Treatment (1 wt %) | Treatment Conditions | PV |
|---|---|---|---|
| 4a | None | — | 58 |
| 4b | Ce(OAc)₃ | 2 days @ 30° C. | 59 |
| 4c | Ce(OAc)₃ | 2 days @ 80° C. | 37 |
| 4d | Fe₃O₄ | 2 days @ 30° C. | 63 |
| 4e | Fe₃O₄ | 2 days @ 80° C. | 41 |
| 4f | Bi(neodecanoate)₃ | 2 days @ 30° C. | 147 |
| 4g | Bi(neodecanoate)₃ | 2 days @ 80° C. | 118 |
| 4h | MAO | 2 days @ 30° C. | 2 |
| 4i | MAO | 2 days @ 80° C. | <1 |

### Reaction 5. Self Metathesis of Soybean Oil and Canola Oil.

Soybean oil and canola oil were treated with either 1 wt % MgAl₂(OⁱPr)₈ or 1 wt % Al(OⁱPr)₃ for 2 hours at 50° C. Both treated oils and untreated oils were subsequently reacted with 1 ppm or 3 ppm of C705 for 2 hours at 50° C., followed by transesterification conditions. Reaction yields were monitored by GC analysis and reported in Table 5. Soybean oil and canola oil self-metathesis equilibrium conversion were defined as 100%−Σ(% methyl palmitate+% methyl stearate+% methyl oleates+% methyl linoleates+% methyl linolenates).

TABLE 5
Self Metathesis of soybean oil and canola oil

| | | | Self-Metathesis Conversion^{a} | |
|---|---|---|---|---|
| Reaction | Seed Oil | Oil Treatment | 1 ppm C705^{b} | 3 ppm C705^{b} |
| 5a | Soybean | None | 45% | 60% |
| 5b | Soybean | Al(OⁱPr)₃ | 60% | 64% |
| 5c | Soybean | MgAl₂(OⁱPr)₈ | 60% | 65% |
| 5d | canola | None | 17% | 46% |
| 5e | canola | Al(OⁱPr)₃ | 48% | 57% |
| 5f | canola | MgAl₂(OⁱPr)₈ | 46% | 58% |

^{a}Reported as GC area percent
^{b}Equilibrium conversion of metathesized soybean oil and canola oil is 67%

Two important observations can be made from Table 5: First, the soluble metal salts reduce the impurities that inhibit the metathesis catalyst. Second, the soluble metal salts do not inhibit the metathesis catalyst.

### Reaction 6. Hexenolysis of Camelina Oil

Samples of camelina oil (refined & bleached (RB), bleached (B), and deodorized (D)) were treated with 1 wt % of MgAl₂(OⁱPr)₈ at 50° C. for 2 hours. Camelina oils RB, B, and D have on average 5.1 double bonds per triglyceride and 1.7 doubles bonds per FAME. The untreated and treated samples were than reacted with 10 ppm, 25 ppm, or 50 ppm C831 and 3 eq/db of 1-hexene at 30° C. for 1 hour. These reactions were transesterified prior to GC analysis. The reaction mixtures were analyzed by GC and the results reported in Table 6.

Due to the complex product mixture produced from metathesis, the extent of conversion was defined as the ratio of methyl 9-decenoate (Me9DA) to methyl palmitate (C16:0). Methyl palmitate is inert to metathesis and therefore was used as an internal standard. Untreated camelina oil reactions produced poor metathesis conversions. MgAl₂(OⁱPr)₈ treated camelina oil samples all underwent hexenolysis (cross metathesis with 1-hexene). The treated samples were approaching equilibrium conversion (i.e., Me9DA/C16:0=4) under these reaction conditions.

TABLE 6
Hexenolysis of Camelina Oil

| | | | Hexenolysis Conversion^{a} | | |
|---|---|---|---|---|---|
| | Camelina | | 10 ppm | 25 ppm | 50 ppm |
| Reaction | Oil | Oil Treatment | C831 | C831 | C831 |
| 6a | RB | None | 0 | 0 | 0.1 |
| 6b | RB | MgAl₂(OⁱPr)₈ | 3.6 | 4.0 | 3.9 |
| 6c | B | None | 0 | 0 | 0.9 |
| 6d | B | MgAl₂(OⁱPr)₈ | 3.8 | 4.0 | 3.9 |
| 6e | D | None | 0 | 0 | 0.2 |
| 6f | D | MgAl₂(OⁱPr)₈ | 3.6 | 4.0 | 3.8 |

^{a}Me9DA/C16:0 ratio determine from GC results

### Reaction 7. Hexenolysis of Soybean Oil

Soybean oil (SBO) was treated with soluble metal salts as reported in Table 7, the soluble metal salts were not removed prior to metathesis. Both the untreated and treated SBO samples were subjected to hexenolysis (3 mol of 1-hexene/mol of SBO double bonds) with either 5 ppm C831 (30° C., 1 hour) or 5 ppm C827 (50° C., 2 hours). These reactions were subjected to transesterification conditions prior to GC analysis. Conversions are reported as the ratio of Me9DA/C16:0, equilibrium conversion was reached at a Me9DA/C16:0 ratio of 2.4.

MgAl₂(OⁱPr)₈, Ti(OⁱPr)₄, and MAO treated SBO reactions all underwent hexenolysis (Table 7), yielding Me9DA/C16:0 ratios of 1.6 to 2.4. Untreated SBO and Bi(neodecanoate)₃ treated SBO resulted in poor hexenolysis conversions, yielding Me9DA/C16:0 ratios of 0.0 to 0.4.

TABLE 7
Hexenolysis of SBO

| | | | | Hexenolysis | |
|---|---|---|---|---|---|
| Reac- | | Treatment | | conversion^{a} | |
| tion | SBO Treatment | Conditions | PV | C831^{b} | C827^{c} |
| 7a | None | | 58 | 0 | 0 |
| 7b | MgAl₂(OⁱPr)₈^{d} | 1 day @ 30° C. | — | 1.6 | 1.6 |
| 7c | MgAl₂(OⁱPr)₈^{d} | 1 day @ 80° C. | — | 2.1 | 2.1 |
| 7d | MgAl₂(OⁱPr)₈^{d} | 4 days @ 30° C. | 1 | 1.8 | 1.9 |
| 7e | MgAl₂(OⁱPr)₈^{d} | 4 days @ 80° C. | <1 | 2.2 | 2.2 |
| 7f | Ti(OⁱPr)₄^{e} | 1 day @ 30° C. | NA^{f} | 2.2 | 2.2 |
| 7g | Ti(OⁱPr)₄^{e} | 1 day @ 80° C. | NA^{f} | 2.3 | 2.3 |
| 7h | Ti(OⁱPr)₄^{e} | 3 days @ 30° C. | NA^{f} | 2.4 | 2.3 |
| 7i | Ti(OⁱPr)₄^{e} | 3 days @ 80° C. | NA^{f} | 2.4 | 2.3 |
| 7j | MAO^{g} | 1 day @ 30° C. | 1 | 1.4 | 1.8 |
| 7k | MAO^{g} | 1 day @ 80° C. | <1 | 2.1 | 2.2 |
| 7l | MAO^{g} | 5 days @ 30° C. | 2 | 2.1 | 2.1 |
| 7m | MAO^{g} | 5 days @ 80° C. | <1 | 2.2 | 2.2 |
| 7n | Bi(neodecanoate)₃^{e} | 1 day @ 30° C. | 147 | 0 | 0 |
| 7o | Bi(neodecanoate)₃^{e} | 1 day @ 80° C. | 117 | 0 | 0 |
| 7p | Bi(neodecanoate)₃^{e} | 3 days @ 30° C. | 147 | 0 | 0 |
| 7r | Bi(neodecanoate)₃^{e} | 3 days @ 80° C. | 117 | 0.4 | 0.2 |
| 7s | Ce(OAc)₃^{e} | 2 days @ 30° C. | 59 | 0 | 0 |
| 7t | Ce(OAc)₃^{e} | 2 days @ 80° C. | 37 | 0 | 0 |
| 7u | Fe₃O₄^{e} | 2 days @ 30° C. | 63 | 0 | 0 |
| 7v | Fe₃O₄^{e} | 2 days @ 80° C. | 41 | 0 | 0 |

^{a}ratio of Me9DA:C16:0 as measured by GC (equilibrium conversion = 2.4)
^{b}5 ppm/db of C831 at 30° C. for 1 hour
^{c}5 ppm/db of C827 at 50° C. for 2 hours
^{d}0.5 wt %
^{e}1 wt %
^{f}NA = Not Applicable as the samples were too dark for PV determinations
^{g}1 wt % of a 10 weight % solution of MAO in toluene

### Reaction 8. PV Determination of Metathesis Reagents

Olefinic feedstocks were treated by heating the olefinic feedstock under inert atmosphere for 3 hours at 50° C. with either 2 wt % Al(OiPr)₃ or 2 wt % Ti(OiPr)₄. In all cases, Al(OiPr)₃ and Ti(OiPr)₄ reduced PV of the starting olefin.

TABLE 8
PV Determinations of Reagents Treated with Soluble Salts

| | Olefinic | Initial PV | PV after | PV after |
|---|---|---|---|---|
| Example | Feedstock | (No Treatment) | Al(OⁱPr)₃ | Ti(OⁱPr)₄ |
| 8a | COD | 9 | <1 | <1 |
| 8b | N-Boc | 12 | <1 | <1 |
| | diallylamine | | | |
| 8c | 9C₁₈ | 8 | 1 | <1 |
| 8d | IC₁₂ | 28 | 3 | <1 |
| 8e | Me9DDA | 203 | 9 | 3 |

Reaction 9. Treatment and ROMP of Dicyclopentadiene (DCPD) with MgAl₂(OⁱPr)₈ and Ti(OⁱPr)₄

DCPD containing 5% cyclopentadiene trimer was treated with 1 wt % Al(OⁱPr)₃ at 50° C. for 3 days. Ultrene 99% DCPD was treated with 1 phr MgAl₂(OⁱPr)₈ or Ti(OⁱPr)₄ at 50° C. for 3 days. The PV of the untreated and treated DCPD are reported in Table 9. MgAl₂(OⁱPr)₈ and Al(OⁱPr)₃ treated DCPD were effective at reducing PV. ROMP conditions: metathesis catalysts C827 (30K:1), 40° C. oven.

TABLE 9
Treatment and ROMP of DCPD with Soluble Metal Salts

| | | | | Time to | Peak |
|---|---|---|---|---|---|
| | Olefin | Additive | | Exotherm | Exotherm |
| Reaction | substrate | (1 phr) | PV | (min) | Temp (° C.) |
| 9a | DCPD resin | none | 2 | ND | ND |
| | with 5% | | | | |
| | trimer | | | | |
| 9b | DCPD resin | Al(OⁱPr)₃ | 0 | ND | ND |
| | with 5% | | | | |
| | trimer | | | | |
| 9c | 99% DCPD | none | 4 | 39.4 | 165° C. |
| 9d | 99% DCPD | MgAl₂(OⁱPr)₈ | <1 | 1.8 | 151° C. |
| 9e | 99% DCPD | Ti(OⁱPr)₄ | <1 | 5.1 | 192° C. |

Reaction 10. Ring-Opening Cross Metathesis (ROCM) of 1,5-Cyclooctadiene (COD) with 1-Hexene

To a 20 mL scintillation vial containing a septum cap and a Teflon-coated flee stir bar was added 1.0 g (9.3 mmol) COD (untreated or treated) and 3.0 g (35.7 mmol) 1-hexene (1-hexene was filtered through alumina) The COD was treated with MgAl₂(OⁱPr)₈ and Ti(OⁱPr)₄ soluble metal complex. The vials were capped and sparged with Argon for 3 minutes.

Metathesis catalyst C848 (100 ppm, 400 μL of 2.3 mM C848 in dichloromethane) was added and the reaction heated to 30° C. The metathesis product mixture was treated with THMP and analyzed by GC at 1 hour and 3 hours (Table 10). Although untreated COD yielded poor conversions (10a), treating COD with 2 wt % Al(OⁱPr)₃ and Ti(OⁱPr)₄, for 3 h at 50° C. under an inert atmosphere underwent complete conversions (10b and 10c).

TABLE 10
ROCM of COD with 1-Hexene

% ROMP product

| Reaction | COD Treatment^{a} | PV | 1 hour | 3 hour |
|---|---|---|---|---|
| 10a | None | 9 | 11 | 11 |
| 10b | 2 wt % Al(OⁱPr)₃ | <1 | 98 | 99 |
| 10c | 2 wt % Ti(OⁱPr)₄ | <1 | 100 | 100 |

^{a}COD treated for 3 hours at 50° C. under an inert atmosphere

### Reaction 11. ROMP of COD

An NMR tube was charged with 460 μL CD₂Cl₂, 25 mg (0.23 mmol) COD (treated or untreated) and 100 ppm metathesis catalyst C848 (20 μL of 1.2 mM C848 in dichloromethane). COD was treated with 2 wt % Al(OⁱPr)₃ and Ti(OⁱPr)₄, for 3 h at 50° C. under an inert atmosphere. The NMR tube was heated in a 30° C. sand bath followed by ¹H NMR analysis after 1 hour and 3 hours. Conversion was determined by comparing the ratio of the integration of the methylene protons in the starting material (δ 2.36 m) with those in the product (δ 2.09 brm and δ 2.04 brm), results are reported in Table 11.

Untreated COD yielded poor polymerization conversion (11a), while both Ti(OⁱPr)₄ and Al(OⁱPr)₃ treated COD samples resulted in significant polymerization conversions (11b and 11c), >80% after 1 hr. Ti(OⁱPr)₄ performed better then Al(OⁱPr)₃ in this application.

TABLE 11
ROMP of COD

% ROMP product

| Reaction | COD Treatment^{a} | PV | 1 hour | 3 hour |
|---|---|---|---|---|
| 11a | None | 9 | 2 | 1 |
| 11b | 2 wt % Al(OⁱPr)₃ | <1 | 81 | 88 |
| 11e | 2 wt % Ti(OⁱPr)₄ | <1 | 99 | 100 |

^{a}COD treated for 3 hours at 50° C. under an inert atmosphere

### Reaction 12: Cross Metathesis of Methyl-9-Dodecenoate (Me9DDA) and 5-Decene (5C₁₀)

To a 20 mL scintillation vial containing a septum cap and a Teflon-coated flee stir bar was added 1.0 g (4.72 mmol) untreated or treated methyl-9-dodecenoate and 2.0 g (14.3 mmol) 5-decene. The vial was capped and sparged with Argon for 3 minutes, 50 ppm metathesis catalyst C848 (200 μL of 1.2 mM C848 in dichloromethane) was added and heated to 30° C. for 3 hours. The reaction was analyzed after 1 hr and 3 hr by treating an aliquot of the reaction mixture with THMP prior to GC analysis. The progress of the reaction was determined my measuring the GC area of the cross metathesis product methyl-9-tetradecenoate (Me9TDA), results are reported in Table 12.

Untreated Me9DDA did not undergo significant cross metathesis while Me9DDA and 5C₁₀ treated with soluble metal salts resulted in up to 21% Me9TDA yields.

TABLE 12
Cross Metathesis of Me9DDA and 5C₁₀

| | |
|---|---|
| Me9DDA/5C₁₀ | % Me9TDA |

| Reaction | Treatment^{a} | PV | 1 hour | 3 hour |
|---|---|---|---|---|
| 12a | None | 203 | <1 | <1 |
| 12b | 2 wt % Al(OⁱPr)₃ | 9 | 10 | 15 |
| 12c | 2 wt % Ti(OⁱPr)₄ | 3 | 15 | 21 |

^{a}Me9DDA and 5C₁₀ were treated for 3 hours at 50° C. under an inert.

### Reaction 13. Alpha Olefin (AO) to Internal Olefin (IO)

To a 15 mL round bottomed flask containing a Teflon-coated flee stir bar was added 1-dodecene (1C₁₂, 1.0 g, 5.95 mmol). A condenser was attached and the system flushed with Argon. 50 ppm metathesis catalyst C848 (250 μL of 1.2 mM C848 in dichloromethane) was added; vacuum was applied to the system through a needle in the septa and heated to 30° C. for 3 hour. Vacuum was provided by a KNF Teflon diaphragm pump (Model UN840.3FTP). The GC results are reported in Table 14, the metathesis product from the self-metathesis of 1C₁₂ is 11-docosene (11C₂₂). Al(OⁱPr)₃ and Ti(OⁱPr)₄ treated 1C₁₂ produced higher yields of 11C₂₂ then untreated 1C₁₂.

TABLE 14
Synthesis of 11C₂₂

% 11C₂₂

| Reaction | 1C₁₂ Treatment^{a} | PV | 1 hour | 3 hour |
|---|---|---|---|---|
| 14a | None | 203 | 18 | 28 |
| 14b | 2 wt % Al(OⁱPr)₃ | 9 | 35 | 50 |
| 14c | 2 wt % Ti(OⁱPr)₄ | 3 | 32 | 42 |

^{a}1C₁₂ was treated for 3 hours at 50° C. under an inert

Reaction 15. Alkenolysis of 9-Octadecene (9C₁₈) with 1-Hexene (1C₆)

To a 20 mL scintillation vial containing a septum cap and a Teflon-coated flee stir bar was added 9C₁₈ (1.0 g, 3.97 mmol) treated with Al(OⁱPr)₃ or Ti(OⁱPr)₄ or untreated 9C₁₈. The vial was capped and sparged with Argon for 3 minutes, 9 ppm of metathesis catalyst C848 (30 μL of 1.2 mM C848 in dichloromethane) was added and the reaction heated to 30° C. The reaction was analyzed after 1 hr and 3 hr by treating an aliquot of the reaction mixture with THMP prior to GC analysis. The yield of 5-tetradecene (5C₁₄) was measured by GC and reported in Table 15. Untreated 1C₁₂ and Al(OⁱPr)₃ treated 1C₁₂ performed poorly, yielding <2% 5C₁₄. Ti(OⁱPr)₄ treated 1C₁₂ produced an 18% to 23% yield of 5C₁₄ under alkenolysis conditions.

TABLE 15
Alkenolysis of 9C₁₈ with 1C₆

GC %5C₁₄

| Reaction | 9C₁₈/1C₆ Treatment^{a} | PV | 1 hour | 3 hour |
|---|---|---|---|---|
| 15a | None | 8 | 0 | 0 |
| 15b | 2 wt % Al(OⁱPr)₃ | 1 | 1 | 2 |
| 15c | 2 wt % Ti(OⁱPr)₄ | <1 | 18 | 24 |

^{a}The 9C₁₈ and 1C₆ mixture was treated for 3 hours at 50° C. under an inert

### Reaction 16: Ring Closing Metathesis (RCM) of N-Boc Diallylamine

To a 40 mL scintillation vial containing a septum cap and a Teflon-coated flee stir bar was added Al(OⁱPr)₃ or Ti(OⁱPr)₄ treated N-Boc-diallylamine (1.0 g, 3.97 mmol) and 580 ppm metathesis catalyst C848 (22 μL of 105 mM C848 in dichloromethane) or 131 ppm metathesis catalyst C848 (22 μL of 23.6 mM C848 in dichloromethane). The vial was capped and vacuum applied through a needle in the septa cap. The reaction was stirred vigorously at 30° C. Vacuum was provided by a KNF Teflon diaphragm pump (Model UN840.3FTP). The conversion of N-Boc-diallylamine to N-Boc-3-pyrroline (NB3P) was measured by GC and reported in Table 16. Untreated and Al(OⁱPr)₃ treated N-Boc diallylamine reactions performed equally poorly under RCM conditions. At higher catalyst loading (580 ppm C848), N-Boc-diallylamine treated with Ti(OⁱPr)₄ produced marginally higher yields of NB3P.

TABLE 16
RCM of N-Boc Diallylamine

| | N-Boc | | GC % NB3P | | GC % NB3P | |
|---|---|---|---|---|---|---|
| | diallylamine | | 131 ppm C848 | | 580 ppm C848 | |
| Reaction | Treatment^{a} | PV | 1 hour | 3 hour | 1 hour | 3 hour |
| 16a | None | 12 | 2 | 3 | 12 | 15 |
| 16b | 2 wt % Al(OⁱPr)₃ | <1 | 1 | 2 | 12 | 15 |
| 16c | 2 wt % Ti(OⁱPr)₄ | <1 | 4 | 6 | 20 | 22 |

^{a}N-Boc diallyamine was treated for 3 hours at 50° C. under an inert

### Reaction 17: Ethenolysis of 9C₁₈ to Produce 1C₁₀

An 88 mL Fischer-Porter bottle (pressure rated to 225 psi) was charged with 10 g (39.7 mmol) 9-octadecene and 1000 ppm C848 (680 μL of 58 mM C848 in dichloromethane). The vessel was sparged with ethylene for 1 minute, heated to 30° C. under 150 psi ethylene for 3 hr. The conversion of 9C₁₈ to 1-decene was determined by GC. In the case of ethenolysis, the presence of either Al(OⁱPr)₃ or Ti(OⁱPr)₄ was detrimental to the desired metathesis reaction (17b and 17c). Ethenolysis of untreated 9C₁₈ (17a) performed better than the treated samples (17b and 17c), see Table 17.

TABLE 17
Ethenolysis of 9C₁₈ to Produce 1C₁₀

GC %1C₁₀

| Reaction | 9C₁₈/1C₆ Treatment^{a} | PV | 1 hour | 3 hour |
|---|---|---|---|---|
| 17a | None | 8 | 12 | 14 |
| 17b | 2 wt % Al(OⁱPr)₃ | 1 | 2 | 3 |
| 17c | 2 wt % Ti(OⁱPr)₄ | <1 | 1 | 2 |

^{a}9C₁₈ was treated for 3 hours at 50° C. under an inert

The claimed invention is:

1. A method for improving the olefin metathesis of an olefinic feedstock, comprising:
providing an olefinic feedstock;
combining the olefinic feedstock with at least one soluble metal salt to form an olefinic feedstock composition;
subjecting the olefinic feedstock composition to conditions effective to reduce the concentration of at least one impurity in the olefinic feedstock;
combining the olefinic feedstock composition with at least one olefin metathesis catalyst; and
subjecting the olefinic feedstock composition to conditions effective to promote an olefin metathesis reaction.

2. The method of claim 1, wherein the olefinic feedstock comprises at least one olefin metathesis active compound derived from petroleum sources, fermentation sources, or natural sources such as oils extracted from plants or animals.

3. The method of claim 2, wherein the at least one olefin metatheis active compound is selected from alpha-olefins, internal olefins, cyclic olefins, polycyclic olefins, fumarates, isobutylene, fatty acids, fatty esters, terpenes, isoprenoids, citronellene, linalool, myrcene, β-pinenespyrethrins, steroids, estolides, alkylresourcinols, cardanol, fatty alcohols, unsaturated triglyceride-based oils, polyunsaturated triglyceride-based oils, or combinations thereof.

4. The method of claim 1, wherein the olefinic feedstock comprises at least one natural seed oil.

5. The method of claim 4, wherein the at least one natural seed oil is selected from soybean oil, camelina oil, sunflower oil, canola oil, safflower oil, cottonseed oil, castor oil, rapeseed oil, peanut oil, corn oil, olive oil, palm oil, sesame oil, grape seed oil, fatty acid methyl esters of natural seed oil, or combinations thereof.

6. The method of claim 1, wherein the at least one soluble metal salt is selected from aluminum isopropoxide (Al(OⁱPr)₃), magnesium aluminum isopropoxide (MgAl₂(OⁱPr)₈), titanium (IV) isopropoxide (Ti(OⁱPr)₄), and methylaluminoxane (MAO), bismuth neodecanoate (Bi(Neodec)₃), cerium acetate hydrate (Ce(OAc)₃×H₂O), or combinations thereof.

7. The method of claim 1, wherein the at least one impurity is a peroxide.

8. The method of claim 1, wherein the olefin metathesis reaction is selected from ring-closing metathesis (RCM), ring-opening metathesis polymerization (ROMP), cross metathesis (CM), ring-opening cross metathesis (ROCM), self-metathesis, alkenolysis, acyclic diene metathesis polymerization, or combinations thereof.

9. The method of claim 1, wherein the at least one olefin metathesis catalyst is a Group 8 transition metal complex having the structure of formula (I) 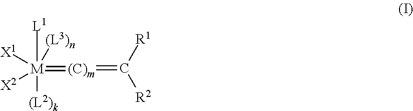
in which:
M is a Group 8 transition metal;
L¹, L², and L³ are independently selected from neutral electron donor ligands;
n is 0 or 1, such that L³ may or may not be present;
m is 0, 1, or 2;
k is 0 or 1;
X¹ and X² are independently anionic ligands; and
R¹ and R² are independently selected from hydrogen, hydrocarbyl, substituted hydrocarbyl, heteroatom-containing hydrocarbyl, substituted heteroatom-containing hydrocarbyl, and functional groups; wherein one or both of R¹ and R² may have the stru-ture —(W)ₙ—U⁺V⁻, in which W is selected from hydrocarbylene, substituted hydrocarbylene, heteroatom-containing hydrocarbylene, or substituted heteroatom-containing hydrocarbylene; U is a positively charged Group 15 or Group 16 element substituted with hydrogen, hydrocarbyl, substituted hydrocarbyl, heteroatom-containing hydrocarbyl, or substituted heteroatom-containing hydrocarbyl; V is a negatively charged counterion; and n is zero or 1,
wherein any two or more of X¹, X², L¹, L², L³, R¹, and R² can be taken together to form one or more cyclic groups, and further wherein any one or more of X¹, X², L¹, L², L³, R¹, and R² may be attached to a support.

10. The method of claim 9, wherein at least one of L¹, L², and L³ is an N-heterocyclic carbene ligand.

11. The method of claim 1, wherein the at least one olefin metathesis catalyst has the structure 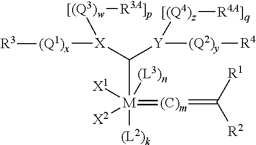
wherein,
M is a Group 8 transition metal;
n is 0 or 1;
m is 0, 1, or 2;
k is 0 or 1;
X¹ and X² are independently selected from anionic ligands;
L² and L³ are independently selected from neutral electron donor ligands, or may be taken together to form a single bidentate electron-donating heterocyclic ligand;
R¹ and R² are independently selected from hydrogen, hydrocarbyl, substituted hydrocarbyl, heteroatom-containing hydrocarbyl, substituted heteroatom-containing hydrocarbyl, and functional groups;
X and Y are independently selected from C, N, O, S, and P;
p is zero when X is O or S, and p is 1 when X is N or P;
q is zero when Y is O or S, and q is 1 when Y is N or P;
Q¹, Q², Q³, and Q⁴ are independently selected from hydrocarbylene, substituted hydrocarbylene, heteroatom-containing hydrocarbylene, substituted heteroatom-containing hydrocarbylene- and —(CO)—, and further wherein two or more substituents on adjacent atoms within Q may be linked to form an additional cyclic group;
w, x, y, and z are independently zero or 1; and
R³, R^{3A}, R⁴, and R^{4A} are independently selected from hydrogen, hydrocarbyl, substituted hydrocarbyl, heteroatom-containing hydrocarbyl, and substituted heteroatom-containing hydrocarbyl,
wherein any two or more of X¹, X², L², L³, R¹, R², Q¹, Q², Q³, Q⁴, R³, R^{3A}, R⁴, and R^{4A} can be taken together to form a cyclic group, and further wherein any one or more of X¹, X², L², L³, Q¹, Q², Q³, Q⁴, R¹, R², R³, R^{3A}, R⁴, and R^{4A} may be attached to a support.

12. The method of claim 11, wherein M is ruthenium, w, x, y, and z are zero, X and Y are N, and R^{3A} and R^{4A} are linked to-form -Q-, such that the complex has the structure 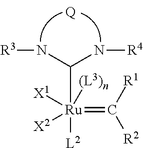
wherein Q is a hydrocarbylene, substituted hydrocarbylene, heteroatom-containing hydrocarbylene, or substituted heteroatom-containing hydrocarbylene linker, and further wherein two or more substituents on adjacent atoms within Q may be linked to form an additional cyclic group.

13. A composition, comprising:
an olefinic feedstock;
at least one soluble metal salt; and
at least one olefin metathesis catalyst, wherein the olefinic feedstock comprises at least one olefin metathesis active compound derived from petroleum sources, fermentation sources, or natural sources such as oils extracted from plants or animals.

14. The composition of claim 13, wherein the at least one olefin metathesis active compound derived from natural sources is at least one natural seed oil.

15. The composition of claim 14, wherein the at least one natural seed oil is selected from soybean oil, camelina oil, sunflower oil, canola oil, safflower oil, cottonseed oil, castor oil, rapeseed oil, peanut oil, corn oil, olive oil, palm oil, sesame oil, grape seed oil, fatty acid methyl esters of natural seed oil, or combinations thereof.

16. The composition of claim 13, wherein the at least one soluble metal salt is selected from aluminum isopropoxide (Al(OⁱPr)₃), magnesium aluminum isopropoxide (MgAl₂(OⁱPr)₈), titanium (IV) isopropoxide (Ti(OⁱPr)₄), and methylaluminoxane (MAO), bismuth neodecanoate (Bi(Neodec)₃), cerium acetate hydrate (Ce(OAc)₃ xH₂O), or combinations thereof.

17. The composition of claim 13, wherein the at least one olefin metathesis catalyst is a Group 8 transition metal complex having the structure of formula (I) 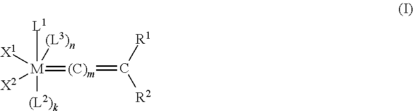
in which:
M is a Group 8 transition metal;
L¹, L², and L³ are independently selected from neutral electron donor ligands;
n is 0 or 1, such that L³ may or may not be present;
m is 0, 1, or 2;
k is 0 or 1;
X¹ and X² are independently anionic ligands; and
R¹ and R² are independently selected from hydrogen, hydrocarbyl, substituted hydrocarbyl, heteroatom-containing hydrocarbyl, substituted heteroatom-containing hydrocarbyl, and functional groups; wherein one or both of R¹ and R² may have the stru-ture —(W)ₙ—U⁺V⁻, in which W is selected from hydrocarbylene, substituted hydrocarbylene, heteroatom-containing hydrocarbylene, or substituted heteroatom-containing hydrocarbylene; U is a positively charged Group 15 or Group 16 element substituted with hydrogen, hydrocarbyl, substituted hydrocarbyl, heteroatom-containing hydrocarbyl, or substituted heteroatom-containing hydrocarbyl; V is a negatively charged counterion; and n is zero or 1,
wherein any two or more of X¹, X², L¹, L², L³, R¹, and R² can be taken together to form one or more cyclic groups, and further wherein any one or more of X¹, X², L¹, L², L³, R¹, and R² may be attached to a support.

18. The composition of claim 17, wherein at least one of L¹, L², and L³ is an N-heterocyclic carbene ligand.

19. The composition of claim 13, wherein the at least one olefin metathesis catalyst has the structure 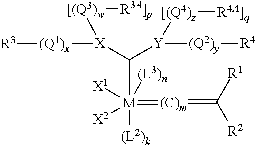
wherein,
M is a Group 8 transition metal;
n is 0 or 1;
m is 0, 1, or 2;
k is 0 or 1;
X¹ and X² are independently selected from anionic ligands;
L² and L³ are independently selected from neutral electron donor ligands, or may be taken together to form a single bidentate electron-donating heterocyclic ligand;
R¹ and R² are independently selected from hydrogen, hydrocarbyl, substituted hydrocarbyl, heteroatom-containing hydrocarbyl, substituted heteroatom-containing hydrocarbyl, and functional groups;
X and Y are independently selected from C, N, O, S, and P;
p is zero when X is O or S, and p is 1 when X is N or P;
q is zero when Y is O or S, and q is 1 when Y is N or P;
Q¹, Q², Q³, and Q⁴ are independently selected from hydrocarbylene, substituted hydrocarbylene, heteroatom-containing hydrocarbylene, substituted heteroatom-containing hydrocarbylene- and —(CO)—, and further wherein two or more substituents on adjacent atoms within Q may be linked to form an additional cyclic group;
w, x, y, and z are independently zero or 1; and
R³, R^{3A}, R⁴, and R^{4A} are independently selected from hydrogen, hydrocarbyl, substituted hydrocarbyl, heteroatom-containing hydrocarbyl, and substituted heteroatom-containing hydrocarbyl,
wherein any two or more of X¹, X², L², L³, R¹, R², Q¹, Q², Q³, Q⁴, R³, R^{3A}, R⁴, and R^{4A} can be taken together to form a cyclic group, and further wherein any one or more of X¹, X², L², L³, Q¹, Q², Q³, Q⁴, R¹, R², R³, R^{3A}, R⁴, and R^{4A} may be attached to a support.

20. The method of claim 19, wherein M is ruthenium, w, x, y, and z are zero, X and Y are N, and R^{3A} and R^{4A} are linked to-form -Q-, such that the complex has the structure 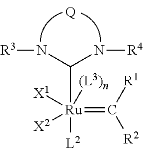
wherein Q is a hydrocarbylene, substituted hydrocarbylene, heteroatom-containing hydrocarbylene, or substituted heteroatom-containing hydrocarbylene linker, and further wherein two or more substituents on adjacent atoms within Q may be linked to form an additional cyclic group.
